(19) United States
(12) Patent Application Publication
(10) Pub. No.: US 2024/0180964 A1
(43) Pub. Date: Jun. 6, 2024

(54) CHIMERIC CYTOKINE RECEPTORS AND USES THEREOF IN CELLULAR THERAPIES

(71) Applicant: Nanjing Legend Biotech Co., Ltd., Nanjing (CN)

(72) Inventors: Qingling JIANG, Nanjing (CN); Yafeng ZHANG, Nanjing (CN); Yanliang ZHU, Nanjing (CN); Miaomiao SHI, Nanjing (CN); Shu WU, Nanjing (CN)

(21) Appl. No.: 18/286,684

(22) PCT Filed: Apr. 19, 2022
(86) PCT No.: PCT/CN2022/087572

§ 371 Date: Oct. 12, 2023

(30) Foreign Application Priority Data
Apr. 19, 2021 (WO) PCT/CN2021/088111

(51) Int. Cl. A61K 35/17; A61K 39/00; A61P 35/00; C07K 16/28; C12N 5/0783

(52) U.S. Cl. CPC A61K 35/17; A61K 39/4611; A61K 39/4631; A61K 39/464429; A61P 35/00; C07K 16/2851; C07K 16/2896; C12N 5/0636; A61K 2239/13; A61K 2239/17; A61K 2239/21; A61K 2239/22; A61K 2239/48

(57) ABSTRACT

An immune effector cell expressing a chimeric cytokine receptor comprising a first extracellular antigen binding domain, a first transmembrane domain, and a cytokine receptor intracellular domain; and a functional exogenous receptor comprising a second extracellular antigen binding domain, a second transmembrane domain, and an intracellular signaling domain.

### CROSS REFERENCE

This application claims benefit of priority of International Patent Application No. PCT/CN2021/088111 filed on Apr. 19, 2021, the content of which is incorporated herein by reference in its entirety.

### SEQUENCE LISTING

This application incorporates by reference a Sequence Listing submitted with this application as a text format, entitled “P11132-PCT.220419.Sequence Listing.txt,” created on Apr. 15, 2022 having a size of 10,10,034 bytes.

### 1. FIELD

The present disclosure relates to chimeric cytokine receptors, engineered immune effector cells comprising same, and methods of use thereof. The present disclosure further relates to activation and expansion of cells for therapeutic uses, especially to functional exogenous receptors (such as chimeric antigen receptors)-based T cell immunotherapies.

### 2. BACKGROUND

Adoptive transfer of T cells represents an emerging innovative therapeutic strategy against cancer. Nevertheless, various barriers restrict the efficacy and/or prevent the widespread use of CAR-T cell therapies particularly in solid tumors. There are four CAR-T products approved by the FDA, they are all autologous CAR-T cells that are derived from the subject's own T cells. Allogeneic CAR-T cells derived from healthy donors offers a more commercially viable off-the-shelf option with the potential to treat a broader range of subjects. Although allogeneic CAR-T cells has many advantages over autologous CAR-T cells, the durability of the responses to the former was shorter than that the latter. Thus, there is a need in the art for the next-generation allogeneic CAR-T cells with improved persistence.

### 3. SUMMARY

In one aspect, provided herein is an immune effector cell expressing (i) a chimeric cytokine receptor comprising (a) a first extracellular antigen binding domain, (b) a first transmembrane domain, and (c) a cytokine receptor intracellular domain; and (ii) optionally a functional exogenous receptor comprising (a) a second extracellular antigen binding domain, (b) a second transmembrane domain, and (c) an intracellular signaling domain.

In one aspect, provided herein is an immune effector cell expressing (i) a chimeric cytokine receptor comprising (a) a first transmembrane domain, and (b) a cytokine receptor intracellular domain; and (ii) a functional exogenous receptor comprising (a) an extracellular antigen binding domain, (b) a second transmembrane domain, and (c) an intracellular signaling domain.

In some embodiments, an immune effector cell expressing (i) a chimeric cytokine receptor comprising (a) a first extracellular antigen binding domain, wherein the first extracellular antigen binding domain is derived from NKG2D, truncated NKG2D, antibody or antigen binding fragment thereof targeting NKG2D ligands, TIGIT or SIRP-α, or a variant thereof, (b) a first transmembrane domain, and (c) a cytokine receptor intracellular domain; and (ii) optionally a functional exogenous receptor comprising (a) a second extracellular antigen binding domain, (b) a second transmembrane domain, and (c) an intracellular signaling domain. In some embodiments, an immune effector cell expressing (i) a chimeric cytokine receptor comprising (a) a first extracellular antigen binding domain, wherein the first extracellular antigen binding domain is derived from NKG2D, truncated NKG2D, antibody or antigen binding fragment thereof targeting NKG2D ligands, TIGIT or SIRP-α, or a variant thereof, (b) a first transmembrane domain, and (c) a cytokine receptor intracellular domain; and (ii) a functional exogenous receptor comprising (a) a second extracellular antigen binding domain, (b) a second transmembrane domain, and (c) an intracellular signaling domain. In some embodiments, immune effector cell comprises the amino acid sequence of SEQ ID NOs: 29, 135, 141, 143 or 149 or an amino acid sequence having at least 75%, 80%, 85%, 90%, 91%, 92%, 93%, 94%, 95%, 96%, 97%, 98%, or 99% identity to SEQ ID NOs: 29, 135, 141,143 or 149. In some embodiments, immune effector cell comprising the amino acid sequence of SEQ ID NOs: 30-134, 136-140, 142, 144, 146 or 148, or an amino acid sequence having at least 75%, 80%, 85%, 90%, 91%, 92%, 93%, 94%, 95%, 96%, 97%, 98%, or 99% identity to SEQ ID NOs: 30-134, 136-140, 142, 144, 146 or 148. In some embodiments, immune effector cell comprises the amino acid sequence of SEQ ID NOs:150 or 151, or an amino acid sequence having at least 75%, 80%, 85%, 90%, 91%, 92%, 93%, 94%, 95%, 96%, 97%, 98%, or 99% identity to SEQ ID NOs:150 or 151.

In some embodiments, the immune effector cell further comprises one or more tags. In some embodiments, a tag is linked to chimeric cytokine receptor and/or the functional exogenous receptor. In some embodiments, the tag linking to the chimeric cytokine receptor is different form the tag linking to the functional exogenous receptor. In some embodiments, the tag linking to the chimeric cytokine receptor and/or the functional exogenous receptor comprises an amino acid sequence of SEQ ID NOs: 4, 5 or 158-160, or an amino acid sequence having at least 75%, 80%, 90%, 91%, 92%, 93%, 94%, 95%, 96%, 97%, 98%, or 99% identity to an amino acid sequence selected from SEQ ID NOs: 4, 5 or 158-160.

In some embodiments, the extracellular antigen binding domain binds to an antigen expressed on the surface of a target cell such as a tumor cell. In some embodiments, the extracellular antigen binding domain is derived from NKG2D or truncated NKG2D (such as the ECD of NKG2D or truncated NKG2D), or a variant thereof. In some embodiments, the extracellular antigen binding domain comprises the amino acid sequence of SEQ ID NOs: 6, 156 or 157, or an amino acid sequence having at least 75%, 80%, 85%, 90%, 91%, 92%, 93%, 94%, 95%, 96%, 97%, 98%, or 99% identity to SEQ ID NOs: 6, 156 or 157. In some embodiments, the extracellular antigen binding domain is derived from an NKG2D ligand or a variant thereof. In some embodiments, the NKG2D ligand is selected from a group consisting of MICA/B, ULBP-1, ULBP-2,5,6 and ULBP-3. In some embodiments, the antibody or antigen binding fragment thereof comprises one or more scFv(s) or one or more sdAb(s) that bind to the NKG2D ligand. In some embodiments, the extracellular antigen binding domain is derived from TIGIT or a variant thereof. In some embodiments, the extracelluar antigen domain is derived from the ECD of TIGIT. In some embodiments, the extracellular antigen binding domain comprises the amino acid sequence of SEQ ID NO: 145 or an amino acid sequence having at least 75%, 80%, 85%, 90%, 91%, 92%, 93%, 94%, 95%, 96%, 97%, 98%, or 99% identity to SEQ ID NO: 145. In some embodiments, the extracellular antigen binding domain is derived from SIRP-a or a variant thereof. In some embodiments, the extracelluar antigen domain is derived from the ECD of SIRP-α. In some embodiments, the extracellular antigen binding domain comprises the amino acid sequence of SEQ ID NO: 147 or an amino acid sequence having at least 75%, 80%, 85%, 90%, 91%, 92%, 93%, 94%, 95%, 96%, 97%, 98%, or 99% identity to SEQ ID NO: 147. In some embodiments, the first or the second extracellular antigen binding domain binds to an antigen expressed on the surface of a target cell such as a tumor cell. In some embodiments, the first or the second extracellular antigen binding domain is derived from NKG2D or truncated NKG2D (such as the ECD of NKG2D or truncated NKG2D), or a variant thereof. In some embodiments, the first or the second extracellular antigen binding domain comprises the amino acid sequence of SEQ ID NOs: 6, 156 or 157, or an amino acid sequence having at least 75%, 80%, 85%, 90%, 91%, 92%, 93%, 94%, 95%, 96%, 97%, 98%, or 99% identity to SEQ ID NOs: 6, 156 or 157. In some embodiments, the first or the second extracellular antigen binding domain is derived from an NKG2D ligand or a variant thereof. In some embodiments, the NKG2D ligand is selected from a group consisting of MICA/B, ULBP-1, ULBP-2,5,6 and ULBP-3. In some embodiments, the antibody or antigen binding fragment thereof comprises one or more scFv(s) or one or more sdAb(s) that bind to the NKG2D ligand. In some embodiments, the first or the second extracellular antigen binding domain is derived from TIGIT or a variant thereof. In some embodiments, the first or the second extracelluar antigen domain is derived from the ECD of TIGIT. In some embodiments, the first or the second extracellular antigen binding domain comprises the amino acid sequence of SEQ ID NO: 145 or an amino acid sequence having at least 75%, 80%, 85%, 90%, 91%, 92%, 93%, 94%, 95%, 96%, 97%, 98%, or 99% identity to SEQ ID NO: 145. In some embodiments, the first or the second extracellular antigen binding domain is derived from SIRP-a or a variant thereof. In some embodiments, the first or the second extracelluar antigen domain is derived from the ECD of SIRP-α. In some embodiments, the first or the second extracellular antigen binding domain comprises the amino acid sequence of SEQ ID NO: 147 or an amino acid sequence having at least 75%, 80%, 85%, 90%, 91%, 92%, 93%, 94%, 95%, 96%, 97%, 98%, or 99% identity to SEQ ID NO: 147.

In some embodiments, the first transmembrane domain comprises a Janus Kinase (JAK)-binding domain. In some embodiments, the JAK-binding domain is derived from a group consisting of EPOR, GHR, TPOR, and a variant thereof. In some embodiments, the JAK-binding domain is derived from TPOR, e.g., comprising an amino acid sequence of SEQ ID NO: 7 or an amino acid sequence having at least 75%, 80%, 85%, 90%, 91%, 92%, 93%, 94%, 95%, 96%, 97%, 98%, or 99% identity to SEQ ID NO:7. In some embodiments, the JAK-binding domain is derived from EPOR. In some embodiments, the JAK-binding domain is derived from GHR.

In some embodiments, the cytokine receptor intracellular domain is derived from one or more cytokine receptors selected from a group consisting of IL2Rα, IL7Rα, IL9R-1, IL9R-2, IL9R-3, IL12Rβ1, IL12Rβ2, IL15Rα, IL15Rβ-1, IL15Rβ-2, IL15Rβ-3, IL18Rα, IL18Rβ, IL21R-1, IL21R-2, IL21R-3, IL10R2, IL22R1, IL23R-1, IL23R-2, TL23R-3, IL27Rα, gp130, IL31RA, OSMRβ, IL36R, IL1RAcP, GM-CSFRα, GM-CSFRβ-1, GM-CSFRβ-2, a variant thereof, and combinations thereof. In some embodiments, the cytokine receptor intracellular domain is selected from IL-7Rα, IL12Rβ1, IL12Rβ2, IL15Rα, IL15Rβ-1, IL15Rβ-2, IL15Rβ-3, IL-18Rα, IL18Rβ, IL21R-1, IL21R-2, IL21R-3, IL23R-1, IL23R-2, TL23R-3, GM-CSFRα, GM-CSFRβ-1, GM-CSFRβ-2, IL9R-1, IL9R-2, IL9R-3, IL-7Rα-IL12Rβ1, IL-7Rα-IL15Rα, IL-7Rα-IL15RP-1, IL-7Rα-IL15Rβ-2, IL7Rα-IL21R-1, IL7Rα-IL21R-2, IL7Rα-IL21R-3, IL7Rα-IL23R-2, IL7Rα-GMCSFRα, IL-7Rα-GM-CSFRβ-1, IL-7Rα-GM-CSFRβ-2, IL12Rβ1-IL15Rα, IL12Rβ1-IL15Rβ-1, IL12Rβ1-IL15Rβ-2, IL12Rβ1-IL-21R-1, IL12Rβ1-IL-21R-2, IL12Rβ1-IL-21R-3, IL12Rβ1-IL-23R-2, IL12Rβ1-GM-CSFRα, IL12Rβ1-GM-CSFRβ-1, IL12Rβ1-GM-CSFRβ-2, IL12Rβ2-IL15Rα, IL12Rβ2-IL15Rβ-1, IL12Rβ2-IL15Rβ-2, IL12Rβ2-IL-21R-1, IL12Rβ2-IL-21R-2, IL12Rβ2-IL-21R-3, IL12Rβ2-IL-23R-2, IL12Rβ2-GM-CSFRα, IL12Rβ2-GM-CSFRβ-1, IL12Rβ2-GM-CSFRβ-2, IL15Rα-IL-21R-1, IL15Rα-IL-21R-2, IL15Rα-IL-21R-3, IL15Rα-IL-23R-2, IL15Rα-GM-CSFRα, IL15Rα-GM-CSFRβ-1, IL15Rα-GM-CSFRβ-2, IL15Rβ-1-IL-21R-1, L15Rβ-1-IL-21R-2, IL15Rβ-1-IL-21R-3, IL15Rβ-1-IL-23R-2, IL15Rβ-1-GM-CSFRα, IL15Rβ-1-GM-CSFRβ-1, IL15Rβ-1-GM-CSFRβ-2, IL15Rβ-2-IL-21R-1, IL15Rβ-2-IL-21R-2, IL15Rβ-2-IL-21R-3, L15Rβ-2-IL-23R-2, IL15Rβ-2-GM-CSFRα, IL15Rβ-2-GM-CSFRβ-1, IL15Rβ-2-GM-CSFRβ-2, IL-21R-1-IL-23R-2, IL-21R-1-GM-CSFRα, IL-21R-1-GM-CSFRβ-1, IL-21R-1-GM-CSFRβ-2, IL-21R-2-IL-23R-2, IL-21R-2-GM-CSFRα, IL-21R-2-GM-CSFRβ-1, IL-21R-2-GM-CSFRβ-2, IL-21R-3-IL-23R-2, IL-21R-3-GM-CSFRα, IL-21R-3-GM-CSFRβ-1, IL-21R-3-GM-CSFRβ-2, IL-23R-2-GM-CSFRα, IL-23R-2-GM-CSFRβ-1, IL-23R-2-GM-CSFRβ-2, IL-7Rα-IL-9R-2, IL12Rβ1-IL-9R-2, IL12Rβ2-IL-9R-2, IL15Rα-IL-9R-2, IL15Rβ-1-IL-9R-2, IL15Rβ-2-IL-9R-2, IL-21R-1-IL-9R-2, IL-21R-2-IL-9R-2, IL-21R-3-IL-9R-2, IL-23R-2-IL-9R-2, GM-CSFRα-IL-9R-2, GM-CSFRβ-1-IL-9R-2, GM-CSFRβ-2-IL-9R-2, IL-7Rα-IL-12Rβ2, or a variant thereof. In some embodiments, the cytokine receptor intracellular domain comprises a region having an amino acid sequence selected from SEQ ID NOs: 8-28, or an amino acid sequence having at least 75%, 80%, 90%, 91%, 92%, 93%, 94%, 95%, 96%, 97%, 98%, or 99% identity to an amino acid sequence selected from SEQ ID NOs: 8-28. In a specific embodiment, the cytokine receptor intracellular domain comprises one or more the amino acid sequences of SEQ ID NO: 8, 10, 14-17 or 20-28.

In some embodiments, the functional exogenous receptor is a T cell receptor (TCR), a chimeric antigen receptor (CAR), a chimeric TCR (cTCR), or a T cell antigen coupler (TAC)-like chimeric receptor. In some embodiments, the functional exogenous receptor is a CAR. In other embodiments, the functional exogenous receptor is a TCR. In some embodiments, functional exogenous receptor is a CAR as described in more detail in Section 5.2.2. For example, the extracellular domain of the CAR can be in any formats including, e.g., a single CAR, dual CAR, tandem CAR, or split CAR. The extracellular domain of the functional exogenous receptor can bind to antigen expressed on a target cell, e.g., a tumor cell. In some embodiments, the CAR binds to a tumor-associated antigen. In some embodiments, the tumor-associated antigen is selected from the group consisting of GPC3, AFP, Claudin 18.2, CD19, CD20, CD22, BCMA, GD2, DLL3, MSLN, CD30, CLL1 and CD33. In some specific embodiments, the CAR provided herein binds to GPC3. In some more specific embodiments, the CAR comprises the amino acid sequence of SEQ ID NO: 29 or 135. In some specific embodiments, the CAR provided herein binds to CD19. In some more specific embodiments, the CAR comprises the amino acid sequence of SEQ ID NO: 141. In some specific embodiments, the CAR provided herein binds to CD33. In some more specific embodiments, the CAR comprises the amino acid sequence of SEQ ID NO: 143.

In some embodiments, the second transmembrane domain is derived from a molecule selected from the group consisting of CD8, CD4, CD28, CD137, CD80, CD86, CD152 and PD1. In some embodiments, the second transmembrane domain is from CD8α or CD28.

In some embodiments, the intracellular signaling domain comprises a primary intracellular signaling domain of an immune effector cell. In some embodiments, the primary intracellular signaling domain is from CD3ζ.

In some embodiments, the intracellular signaling domain comprises a co-stimulatory signaling domain.

In some embodiments, the co-stimulatory signaling domain is derived from a co-stimulatory molecule selected from the group consisting of CD27, CD28, CD137, OX40, CD30, CD40, CD3, LFA-1, ICOS, CD2, CD7, LIGHT, NKG2C, B7-H3, ligands of CD83 and combinations thereof. In some embodiments, the co-stimulatory signaling domain comprises a cytoplasmic domain of CD28 and/or a cytoplasmic domain of CD137.

In some embodiments, the functional exogenous receptor further comprises a hinge domain located between the C-terminus of the extracellular antigen binding domain and the N-terminus of the transmembrane domain. In some embodiments, the hinge domain is from CD8α.

In some embodiments, the chimeric cytokine receptor and/or the functional exogenous receptor further comprises a signal peptide at the N-terminus of the chimeric cytokine receptor and/or the functional exogenous receptor. In some embodiments, the signal peptide is from CD8α.

In some embodiments, the immune effector cell is a T cell, a natural killer (NK) cell, an NK T cell, a macrophage, a peripheral blood mononuclear cell (PBMC), a monocyte, a neutrophil, or an eosinophil. In some embodiments, the T cell is a cytotoxic T cell, a helper T cell, a natural killer T cell, a αβ T cell, or a γδT cell.

In another aspect, provided herein is a polypeptide comprising (i) a chimeric cytokine receptor comprising (a) a first extracellular antigen binding domain, (b) a first transmembrane domain, and (c) a cytokine receptor intracellular domain; and (ii) optionally a functional exogenous receptor comprising (a) a second extracellular antigen binding domain, (b) a second transmembrane domain, and (c) an intracellular signaling domain. In one aspect, provided herein is a polypeptide comprising (i) a chimeric cytokine receptor comprising (a) a first transmembrane domain, and (b) a cytokine receptor intracellular domain; and (ii) a functional exogenous receptor comprising (a) an extracellular antigen binding domain, (b) a second transmembrane domain, and (c) an intracellular signaling domain.

In some embodiments, a polypeptide comprising (i) a chimeric cytokine receptor comprising (a) a first extracellular antigen binding domain, wherein the first extracellular antigen binding domain is derived from NKG2D, truncated NKG2D, antibody or antigen binding fragment thereof targeting NKG2D ligands, TIGIT or SIRP-α, or a variant thereof, (b) a first transmembrane domain, and (c) a cytokine receptor intracellular domain; and (ii) optionally a functional exogenous receptor comprising (a) a second extracellular antigen binding domain, (b) a second transmembrane domain, and (c) an intracellular signaling domain. In some embodiments, a polypeptide comprising (i) a chimeric cytokine receptor comprising (a) a first extracellular antigen binding domain, wherein the first extracellular antigen binding domain is derived from NKG2D, truncated NKG2D, antibody or antigen binding fragment thereof targeting NKG2D ligands, TIGIT or SIRP-α, or a variant thereof, (b) a first transmembrane domain, and (c) a cytokine receptor intracellular domain; and (ii) a functional exogenous receptor comprising (a) a second extracellular antigen binding domain, (b) a second transmembrane domain, and (c) an intracellular signaling domain. In some embodiments, a polypeptide comprises the amino acid sequence of SEQ ID NOs: 29, 135, 141, 143 or 149, or an amino acid sequence having at least 75%, 80%, 85%, 90%, 91%, 92%, 93%, 94%, 95%, 96%, 97%, 98%, or 99% identity to SEQ ID NOs: 29, 135, 141,143 or 149. In some embodiments, a polypeptide comprises the amino acid sequence of ID NOs: 30-134, 136-140, 142, 144, 146, or 148, or an amino acid sequence having at least 75%, 80%, 85%, 90%, 91%, 92%, 93%, 94%, 95%, 96%, 97%, 98%, or 99% identity to SEQ ID NOs: 30-134, 136-140, 142, 144,146 or 148. In some embodiments, a polypeptide comprises further comprises one or more tags. In some embodiments, a tag is linked to chimeric cytokine receptor and/or the functional exogenous receptor. In some embodiments, the tag linking to the chimeric cytokine receptor is different form the tag linking to the functional exogenous receptor. In some embodiments, the tag linking to the chimeric cytokine receptor the tag comprises an amino acid sequence of SEQ ID NOs: 4, 5 or 158-160, or an amino acid sequence having at least 75%, 80%, 90%, 91%, 92%, 93%, 94%, 95%, 96%, 97%, 98%, or 99% identity to an amino acid sequence selected from SEQ ID NOs: 4, 5 or 158-160. In some embodiments, immune effector cell comprises the amino acid sequence of SEQ ID NOs:150 or 151, or an amino acid sequence having at least 75%, 80%, 85%, 90%, 91%, 92%, 93%, 94%, 95%, 96%, 97%, 98%, or 99% identity to SEQ ID NOs:150 or 151.

In some embodiments, the first extracellular antigen binding domain binds to an antigen expressed on the surface of a target cell such as a tumor cell. In some embodiments, the first extracellular antigen binding domain is derived from NKG2D or truncated NKG2D (such as the ECD of NKG2D or truncated NKG2D), or a variant thereof. In some embodiments, the first extracellular antigen binding domain comprises the amino acid sequence of SEQ ID NOs: 6, 156 or 157, or an amino acid sequence having at least 75%, 80%, 85%, 90%, 91%, 92%, 93%, 94%, 95%, 96%, 97%, 98%, or 99% identity to SEQ ID NOs: 6, 156 or 157. In some embodiments, the first extracellular antigen binding domain is derived from truncated NKG2D (amino acid positions 89-216, SEQ ID NO: 157). In some embodiments, the first extracellular antigen binding domain is derived from truncated NKG2D (amino acid positions 98-216, SEQ ID NO: 156). In some embodiments, the first extracellular antigen binding domain comprises the amino acid sequence of SEQ ID NO: 6. In some embodiments, the first extracellular antigen binding domain is derived from TIGIT (such as the ECD of TIGIT). In some embodiments, the first extracellular antigen binding domain comprises the amino acid sequence of SEQ ID NO: 145. In some embodiments, the first extracellular antigen binding domain is derived from SIRP-α (such as the ECD of SIRP-a). In some embodiments, the first extracellular antigen binding domain comprises the amino acid sequence of SEQ ID NO: 147. In some embodiments, the first extracellular antigen binding domain is derived from an antibody or antigen binding fragment thereof targeting NKG2D ligand or a variant thereof. In some embodiments, the NKG2D ligand is selected from a group consisting of MICA/B, ULBP-1, ULBP-2,5,6 and ULBP-3. In some embodiments, the antibody or antigen binding fragment thereof comprises one or more scFv(s) or one or more sdAb(s) that bind to the NKG2D ligand.

In some embodiments, the first transmembrane domain comprises a Janus Kinase (JAK)-binding domain. In some embodiments, the JAK-binding domain is derived from a group consisting of EPOR, GHR, TPOR, or a variant thereof. In some embodiments, the JAK-binding domain is derived from TPOR, e.g., comprising an amino acid sequence of SEQ ID NO: 7 or an amino acid sequence having at least 75%, 80%, 85%, 90%, 91%, 92%, 93%, 94%, 95%, 96%, 97%, 98%, or 99% identity to SEQ ID NO:7. In some embodiments, the JAK-binding domain is derived from EPOR. In some embodiments, the JAK-binding domain is derived from GHR.

In some embodiments, the cytokine receptor intracellular domain is derived from one or more cytokine receptors selected from a group consisting of IL2Rα, IL7Rα, IL9R-1, IL9R-2, IL9R-3, IL12Rβ1, IL12Rβ2, IL15Rα, IL15Rβ-1, IL15Rβ-2, IL15Rβ-3, IL18Rα, IL18Rβ, IL21R-1, IL21R-2, IL21R-3, IL10R2, IL22R1, IL23R-1, IL23R-2, TL23R-3, IL27Rα, gp130, IL31RA, OSMRβ, IL36R, IL1RAcP, GM-CSFRα, GM-CSFRβ-1, GM-CSFRβ-2, a variant thereof, and combinations thereof. In some embodiments, the cytokine receptor intracellular domain is derived from cytokine receptors selected from IL-7Rα, IL12Rβ1, IL12Rβ2, IL15Rα, IL15Rβ-1, IL15Rβ-2, IL15Rβ-3, IL-18Rα, IL18Rβ, IL21R-1, IL21R-2, IL21R-3, IL23R-1, IL23R-2, TL23R-3, GM-CSFRα, GM-CSFRβ-1, GM-CSFRβ-2, IL9R-1, IL9R-2, TL9R-3, IL-7Rα-IL12Rβ1, IL-7Rα-IL15Rα, IL-7Rα-IL15Rβ-1, IL-7Rα-IL15Rβ-2, IL-7Rα-IL21R-1, IL-7Rα-IL21R-2, IL-7Rα-IL21R-3, IL-7Rα-IL23R-2, IL-7Rα-GMCSFRα, IL-7Rα-GM-CSFRβ-1, IL-7Rα-GM-CSFRβ-2, IL12Rβ1-IL15Rα, IL12Rβ1-IL15Rβ-1, IL12Rβ1-IL15Rβ-2, IL12Rβ1-IL-21R-1, IL12Rβ1-IL-21R-2, IL12Rβ1-IL-21R-3, IL12Rβ1-IL-23R-2, IL12Rβ1-GM-CSFRα, IL12Rβ1-GM-CSFRβ-1, IL12Rβ1-GM-CSFRβ-2, IL12Rβ2-IL15Rα, IL12Rβ2-IL15Rβ-1, IL12Rβ2-IL15Rβ-2, IL12Rβ2-IL-21R-1, IL12Rβ2-IL-21R-2, IL12Rβ2-IL-21R-3, IL12Rβ2-IL-23R-2, IL12Rβ2-GM-CSFRα, IL12Rβ2-GM-CSFRβ-1, IL12Rβ2-GM-CSFRβ-2, IL15Rα-IL-21R-1, IL15Rα-IL-21R-2, IL15Rα-IL-21R-3, IL15Rα-IL-23R-2, IL15Rα-GM-CSFRα, IL15Rα-GM-CSFRβ-1, IL15Rα-GM-CSFRβ-2, IL15Rβ-1-IL-21R-1, L15Rβ-1-IL-21R-2, IL15Rβ-1-IL-21R-3, IL15Rβ-1-IL-23R-2, IL15Rβ-1-GM-CSFRα, IL15Rβ-1-GM-CSFRβ-1, IL15Rβ-1-GM-CSFRβ-2, IL15Rβ-2-IL-21R-1, IL15Rβ-2-IL-21R-2, IL15Rβ-2-IL-21R-3, L15Rβ-2-IL-23R-2, IL15Rβ-2-GM-CSFRα, IL15Rβ-2-GM-CSFRβ-1, IL15Rβ-2-GM-CSFRβ-2, IL-21R-1-IL-23R-2, IL-21R-1-GM-CSFRα, IL-21R-1-GM-CSFRβ-1, IL-21R-1-GM-CSFRβ-2, IL-21R-2-IL-23R-2, IL-21R-2-GM-CSFRα, IL-21R-2-GM-CSFRβ-1, IL-21R-2-GM-CSFRβ-2, IL-21R-3-IL-23R-2, IL-21R-3-GM-CSFRα, IL-21R-3-GM-CSFRβ-1, IL-21R-3-GM-CSFRβ-2, IL-23R-2-GM-CSFRα, IL-23R-2-GM-CSFRβ-1, IL-23R-2-GM-CSFRβ-2, IL-7Rα-IL-9R-2, IL12Rβ1-IL-9R-2, IL12Rβ2-IL-9R-2, IL15Rα-IL-9R-2, IL15Rβ-1-IL-9R-2, IL15Rβ-2-IL-9R-2, IL-21R-1-IL-9R-2, IL-21R-2-IL-9R-2, IL-21R-3-IL-9R-2, IL-23R-2-IL-9R-2, GM-CSFRα-IL-9R-2, GM-CSFRβ-1-IL-9R-2, GM-CSFRβ-2-IL-9R-2, IL-7Rα-IL-12Rβ2, or a variant thereof. In some embodiments, the cytokine receptor intracellular domain comprises a region having an amino acid sequence selected from SEQ ID NOs: 8-28, or an amino acid sequence having at least 75%, 80%, 90%, 91%, 92%, 93%, 94%, 95%, 96%, 97%, 98%, or 99% identity to an amino acid sequence selected from SEQ ID NOs: 8-28. In a specific embodiment, the cytokine receptor intracellular domain comprises one or more the amino acid sequences of SEQ ID NO: 8, 10, 14-17 or 20-28.

In some embodiments, the functional exogenous receptor is a T cell receptor (TCR), a chimeric antigen receptor (CAR), a chimeric TCR (cTCR), or a T cell antigen coupler (TAC)-like chimeric receptor. In some embodiments, the functional exogenous receptor is a CAR. In other embodiments, the functional exogenous receptor is a TCR. In some embodiments, the CAR is a single CAR, dual CAR, tandem CAR or split CAR. In some embodiments, the CAR is as described in Section 5.2.2. In some specific embodiments, the CAR provided herein binds to a tumor-associated antigen. In some specific embodiments, the CAR provided herein binds to GPC3, CD19 or CD33. In some more specific embodiments, the CAR provided herein comprises the amino acid sequence of SEQ ID NOs: 29, 135, 141, 143 or 149, or an amino acid sequence having at least 75%, 80%, 85%, 90%, 91%, 92%, 93%, 94%, 95%, 96%, 97%, 98%, or 99% identity to SEQ ID NOs: 29, 135, 141,143 or 149.

In some embodiments, the second transmembrane domain is derived from a molecule selected from the group consisting of CD8, CD4, CD28, CD137, CD80, CD86, CD152 and PD1. In some embodiments, the second transmembrane domain is from CD8α or CD28.

In some embodiments, the intracellular signaling domain comprises a primary intracellular signaling domain of an immune effector cell. In some embodiments, the primary intracellular signaling domain is from CD3ζ.

In some embodiments, the intracellular signaling domain comprises a co-stimulatory signaling domain. In some embodiments, the co-stimulatory signaling domain is derived from a co-stimulatory molecule selected from the group consisting of CD27, CD28, CD137, OX40, CD30, CD40, CD3, LFA-1, ICOS, CD2, CD7, LIGHT, NKG2C, B7-H3, ligands of CD83, and combinations thereof. In some embodiments, the co-stimulatory signaling domain comprises a cytoplasmic domain of CD28 and/or a cytoplasmic domain of CD137.

In some embodiments, the functional exogenous receptor further comprises a hinge domain located between the C-terminus of the extracellular antigen binding domain and the N-terminus of the transmembrane domain. In some embodiments, the hinge domain is from CD8α.

In some embodiments, the chimeric cytokine receptor and/or the functional exogenous receptor further comprises a signal peptide at the N-terminus of the chimeric cytokine receptor and/or the functional exogenous receptor. In some embodiments, the signal peptide is from CD8α.

In some embodiments, the chimeric cytokine receptor and the functional exogenous receptor are linked to each other via a peptide linker. In some embodiments, the peptide linker is a 2A self-cleaving peptide optionally selected from a group consisting of F2A, E2A, P2A, T2A, and variants thereof.

In some embodiments, the polypeptide further comprises one or more tags. In some embodiments, a tag is linked to chimeric cytokine receptor and/or the functional exogenous receptor. In some embodiments, the tag linking to the chimeric cytokine receptor is different form the tag linking to the functional exogenous receptor. In some embodiments, the tag linking to the chimeric cytokine receptor and/or the functional exogenous receptor comprises an amino acid sequence of SEQ ID NOs: 4, 5 or 158-160, or an amino acid sequence having at least 75%, 80%, 90%, 91%, 92%, 93%, 94%, 95%, 96%, 97%, 98%, or 99% identity to an amino acid sequence selected from SEQ ID NOs: 4, 5 or 158-160.

In another aspect, provided herein is an isolated nucleic acid comprising a nucleic acid sequence encoding the polypeptide provided herein. In yet another aspect, provided herein is an isolated nucleic acid comprising (i) a first region encoding a chimeric cytokine receptor comprising (a) a first extracellular antigen binding domain, wherein the first extracellular antigen binding domain is derived from NKG2D, truncated NKG2D, antibody or antigen binding fragment thereof targeting NKG2D ligands, TIGIT, or SIRP-α, or a variant thereof, (b) a first transmembrane domain, and (c) a cytokine receptor intracellular domain; and (ii) optionally a second region encoding a functional exogenous receptor comprising (a) a second extracellular antigen binding domain, (b) a second transmembrane domain, and (c) an intracellular signaling domain. In yet another aspect, provided herein is a vector comprising the isolated nucleic acid provided herein.

In yet another aspect, provided herein is a method of making an immune effector cell comprising introducing into an immune cell the nucleic acid or the vector provided herein. In yet another aspect, provided herein is a method of making an immune effector cell comprising introducing into an immune cell a composition comprising (i) a first nucleic acid encoding a chimeric cytokine receptor comprising (a) a first extracellular antigen binding domain, (b) a first transmembrane domain, and (c) a cytokine receptor intracellular domain; and (ii) optionally a second nucleic acid encoding a functional exogenous receptor comprising (a) a second extracellular antigen binding domain, (b) a second transmembrane domain, and (c) an intracellular signaling domain.

In yet another aspect, provided herein is an immune effector cell produced according the method provided herein.

In yet another aspect, provided herein is a pharmaceutical composition, comprising the immune effector cell, the polypeptide, the nucleic acid, or the vector provided herein, and a pharmaceutically acceptable carrier.

In yet another aspect, provided herein is a method of treating a disease or disorder in a subject, comprising administering to the subject an effective amount of the pharmaceutical composition provided herein.

In yet another aspect, provided herein is a disease or disorder comprising a cancer, an inflammatory or autoimmune disease. In some embodiments, a cancer is solid cancer or hematologic cancer. In some embodiments, a cancer is liver cancer, lymphoma, acute myeloid leukemia (AML) or chronic myelogenous leukemia (CML).

In another aspect, provided herein is a chimeric cytokine receptor comprising: (a) an extracellular antigen binding domain, (b) a transmembrane domain, and (c) a cytokine receptor intracellular domain. In some embodiments, the extracellular antigen binding domain is derived from NKG2D, truncated NKG2D, antibody or antigen binding fragment thereof targeting NKG2D ligands, TIGIT, SIRP-α, or a variant thereof. In some embodiments, the extracellular antigen binding domain is derived from the extracellular domain of NKG2D or a variant thereof, wherein optionally the extracellular antigen binding domain comprises the amino acid sequence of SEQ ID NOs: 6, 156 or 157 or an amino acid sequence having at least 75%, 80%, 85%, 90%, 91%, 92%, 93%, 94%, 95%, 96%, 97%, 98%, or 99% identity to SEQ ID NOs: 6, 156 or 157. In other embodiments, the extracelluar domain comprises an antibody or antigen binding fragment thereof that binds to an NKG2D ligand. In some embodiments, the extracelluar domain comprises one or more scFv(s) or one or more sdAb(s) that bind to an NKG2D ligand. In some embodiments, the NKG2D ligand is selected from a group consisting of MICA/B, ULBP-1, ULBP-2,5,6 and ULBP-3. In some embodiments, the extracellular antigen binding domain is derived from an extracellular domain of an immune checkpoint. In some embodiments, the immune checkpoint is selected from a group consisting of PD-1, CTLA4, or a variant thereof. In some embodiments, the immune checkpoint is PD-1. In some embodiments, the immune checkpoint is CTLA4.

In another aspect, provided herein is an immune effector cell expressing the chimeric cytokine receptor provided herein.

### 4. BRIEF DESCRIPTION OF THE FIGURES

FIGS. 1A-1B show exemplary constructs of CARs or TCRs armored with NKG2D, truncated NKG2D or mutated NKG2D chimeric cytokine receptor. FIG. 1A shows a schematic of a CAR armored with NKG2D, truncated NKG2D or mutated NKG2D chimeric cytokine receptor. FIG. 1B shows a schematic of a TCR NKG2D, truncated NKG2D or mutated NKG2D chimeric cytokine receptor.

FIGS. 2A-2B show exemplary constructs of CARs or TCRs armored with chimeric cytokine receptor which includes a binding domain targeting NKG2D ligands. FIG. 2A shows a schematic of a CAR armored with chimeric cytokine receptor which includes a binding domain targeting NKG2D ligands. FIG. 2B shows a schematic of a TCR armored with chimeric cytokine receptor which includes a binding domain targeting NKG2D ligands.

FIGS. 3A-3B show exemplary constructs of CARs or TCRs armored with different cytokine receptors. FIG. 3A shows a CAR armored with NKG2D or mutated NKG2D chimeric cytokine receptors. FIG. 3B shows a TCR armored with NKG2D or mutated NKG2D chimeric cytokine receptors.

FIG. 4 shows ligand surface expression of NKG2D on HCC cell lines huh7.

FIG. 5 shows long-term cytotoxicity of anti-GPC3 CAR or NKG2D chimeric two cytokine receptors armored anti-GPC3 CAR γδT cells co-culture with huh7 cells.

FIG. 6 shows T cell proliferation in long-term co-cultures of anti-GPC3 CAR T or NKG2D chimeric two cytokine receptors armored anti-GPC3 CAR γδT cells with huh7 cells.

FIG. 7 shows long-term cytotoxicity of NKG2D chimeric single cytokine receptors armored anti-GPC3 CAR γδT cells co-culture with huh7 cells.

FIG. 8 shows T cell proliferation in long-term co-cultures of NKG2D chimeric single cytokine receptors armored anti-GPC3 CAR γδT cells with huh7 cells.

FIG. 9 shows long-term cytotoxicity of NKG2D chimeric two cytokine receptors armored anti-GPC3 CAR γδT cells co-culture with huh7 cells.

FIG. 10 shows T cell proliferation in long-term co-cultures of NKG2D chimeric two cytokine receptors armored anti-GPC3 CAR γδT cells with huh7 cells.

FIG. 11 shows long-term cytotoxicity of NKG2D chimeric two cytokine receptors armored anti-GPC3 CAR γδT cells co-culture with huh7 cells.

FIG. 12 shows T cell proliferation in long-term co-cultures of NKG2D chimeric two cytokine receptors armored anti-GPC3 CAR γδT cells with huh7 cells.

FIG. 13 shows long-term cytotoxicity of NKG2D chimeric two cytokine receptors armored anti-GPC3 CAR αβT cells co-culture with huh7 cells.

FIG. 14 shows T cell proliferation in long-term co-cultures of NKG2D chimeric two cytokine receptors armored anti-GPC3 CAR αβT cells with huh7 cells.

FIG. 15 shows long-term cytotoxicity of NKG2D chimeric two cytokine receptors armored anti-GPC3 CAR αβT cells co-culture with huh7 cells.

FIG. 16 shows T cell proliferation in long-term co-cultures of NKG2D chimeric two cytokine receptors armored anti-GPC3 CAR αβT cells with huh7 cells.

FIG. 17 shows long-term cytotoxicity of truncated NKG2D chimeric two cytokine receptors armored anti-GPC3 CAR αβT cells co-culture with huh7 cells.

FIG. 18 shows T cell proliferation in long-term co-cultures of truncated NKG2D chimeric two cytokine receptors armored anti-GPC3 CAR αβT cells with huh7 cells.

FIG. 19 shows long-term cytotoxicity of anti-MIC-A/B scFv chimeric two cytokine receptors armored anti-GPC3 CAR γδT cells co-culture with huh7 cells.

FIG. 20 shows T cell proliferation in long-term co-cultures of anti-MIC-A/B scFv chimeric two cytokine receptors armored anti-GPC3 CAR γδT cells with huh7 cells.

FIG. 21 shows anti-tumor effect of anti-GPC3 CAR γδT cells or anti-GPC3 CAR γδT cells armored with NKG2D chimeric two cytokine receptors in huh7 xenograft model.

FIG. 22 shows long-term cytotoxicity of NKG2D chimeric two cytokine receptors armored construct 185 CAR αβT cells co-culture with huh7 cells.

FIG. 23 shows T cell proliferation in long-term co-cultures of NKG2D chimeric two cytokine receptors armored construct 185 CAR αβT cells with huh7 cells.

FIG. 24 shows long-term cytotoxicity of NKG2D chimeric two cytokine receptors armored CTL-019 CAR γδT cells co-culture with Raji cells.

FIG. 25 shows T cell proliferation in long-term co-cultures of NKG2D chimeric two cytokine receptors armored CTL-019 CAR γδT cells co-culture with Raji cells.

FIG. 26 shows long-term cytotoxicity of NKG2D chimeric two cytokine receptors armored CTL-019 CAR αβT cells co-culture with Raji cells.

FIG. 27 shows T cell proliferation in long-term co-cultures of NKG2D chimeric two cytokine receptors armored CTL-019 CAR αβT cells co-culture with Raji cells.

FIG. 28 shows long-term cytotoxicity of NKG2D chimeric two cytokine receptors armored AS67190 CAR αβT cells co-culture with U937 cells.

FIG. 29 shows T cell proliferation in long-term co-cultures of NKG2D chimeric two cytokine receptors armored AS67190 CAR αβT cells co-culture with U937 cells.

FIG. 30 shows long-term cytotoxicity of TIGIT chimeric two cytokine receptors armored anti-GPC3 CAR γδT cells co-culture with huh7 cells.

FIG. 31 shows T cell proliferation in long-term co-cultures of TIGIT chimeric two cytokine receptors armored anti-GPC3 CAR γδT cells co-culture with huh7 cells.

FIG. 32 shows anti-tumor effect of anti-GPC3 CAR γδT cells or anti-GPC3 CAR γδT cells armored with TIGIT chimeric two cytokine receptors in huh7 xenograft model.

FIG. 33 shows long-term cytotoxicity of SIRP-α chimeric two cytokine receptors armored AS67190 CAR γδT cells co-culture with U937 cells.

FIG. 34 shows T cell proliferation in long-term co-cultures of SIRP-α chimeric two cytokine receptors armored AS67190 CAR γδT cells co-culture with U937 cells.

FIG. 35 shows T cell proliferation of NKG2D CAR or BM CAR αβT cells in culture.

FIG. 36 shows T cell viability of NKG2D or BM CAR αβT cells in culture.

FIG. 37 shows long-term cytotoxicity of depleted NKG2D or constitutively active chimeric two cytokine receptors armored BM CAR αβT cells co-culture with huh7 cells.

FIG. 38 shows T cell proliferation in long-term co-cultures of depleted NKG2D or constitutively active chimeric two cytokine receptors armored BM CAR αβT cells co-culture with huh7 cells.

### 5. DETAILED DESCRIPTION

The present disclosure is based, in part, on the surprising finding of improved functions and properties of engineered immune cells expressing a chimeric cytokine receptor.

Modified CAR-T cells have been generated to secrete cytokines to promote their survival and/or greater activity. However, the effect of constitutively or inductively secreting cytokines is systemic and not unique to CAR-T cells. They also induce expansion of endogenous immune cells, such as NK cells, NKT cells, dendritic cells (DCs), macrophages and endogenous CD8 T cells (see Avanzi M. P. et al., Cell Rep. 15; 23 (7): 2130-2141 (2015)). Over-activation of the immune system may be fatal, and thus more desirable and safer engineering armors are needed.

The present disclosure provides compositions and methods of genetically modifying immune cells to express chimeric cytokine receptors responsive to a ligand, which specifically expressed in tumor cells. When the chimeric cytokine receptors bind to the ligand, downstream intracellular cytokine signals activate and uniquely improve the functional activities of genetically modified immune cells, which avoids extensive activation of the immune system. The engineered cells provided herein exhibit enhanced proliferation and potency for treatment of, e.g., cancers and infectious diseases.

### 5.1. Definitions

Techniques and procedures described or referenced herein include those that are generally well understood and/or commonly employed using conventional methodology by those skilled in the art, such as, for example, the widely utilized methodologies described in Sambrook et al., Molecular Cloning: A Laboratory Manual (3d ed. 2001); Current Protocols in Molecular Biology (Ausubel et al. eds., 2003); Therapeutic Monoclonal Antibodies: From Bench to Clinic (An ed. 2009); Monoclonal Antibodies: Methods and Protocols (Albitar ed. 2010); and Antibody Engineering Vols 1 and 2 (Kontermann and Dübel eds., 2d ed. 2010). Unless otherwise defined herein, technical and scientific terms used in the present description have the meanings that are commonly understood by those of ordinary skill in the art. For purposes of interpreting this specification, the following description of terms will apply and whenever appropriate, terms used in the singular will also include the plural and vice versa. In the event that any description of a term set forth conflicts with any document incorporated herein by reference, the description of the term set forth below shall control.

The term “antibody,” “immunoglobulin,” or “Ig” is used interchangeably herein, and is used in the broadest sense and specifically covers, for example, monoclonal antibodies (including agonist, antagonist, neutralizing antibodies, full length or intact monoclonal antibodies), antibody compositions with polyepitopic or monoepitopic specificity, polyclonal or monovalent antibodies, multivalent antibodies, multispecific antibodies (e.g., bispecific antibodies so long as they exhibit the desired biological activity), formed from at least two intact antibodies, single chain antibodies, single domain antiboides (e.g., V_{H}H) and fragments thereof (e.g., domain antibodies). An antibody can be human, humanized, chimeric and/or affinity matured, as well as an antibody from other species, for example, mouse, rabbit, llama, etc. The term “antibody” is intended to include a polypeptide product of B cells within the immunoglobulin class of polypeptides that is able to bind to a specific molecular antigen and is composed of two identical pairs of polypeptide chains, wherein each pair has one heavy chain (about 50-70 kDa) and one light chain (about 25 kDa), each amino-terminal portion of each chain includes a variable region of about 100 to about 130 or more amino acids, and each carboxy-terminal portion of each chain includes a constant region. See, e.g., Antibody Engineering (Borrebaeck ed., 2d ed. 1995); and Kuby, Immunology (3d ed. 1997). Antibodies also include, but are not limited to, synthetic antibodies, recombinantly produced antibodies, single domain antibodies including from Camelidae species (e.g., llama or alpaca) or their humanized variants, intrabodies, anti-idiotypic (anti-Id) antibodies, and functional fragments (e.g., antigen-binding fragments) of any of the above, which refers to a portion of an antibody heavy or light chain polypeptide that retains some or all of the binding activity of the antibody from which the fragment was derived. Non-limiting examples of functional fragments (e.g., antigen-binding fragments) include single-chain Fvs (scFv) (e.g., including monospecific, bispecific, etc.), Fab fragments, F(ab′) fragments, F(ab)₂ fragments, F(ab′)₂ fragments, disulfide-linked Fvs (dsFv), Fd fragments, Fv fragments, diabody, triabody, tetrabody, and minibody. In particular, antibodies provided herein include immunoglobulin molecules and immunologically active portions of immunoglobulin molecules, for example, antigen-binding domains or molecules that contain an antigen-binding site that binds to an antigen (e.g., one or more CDRs of an antibody). Such antibody fragments can be found in, for example, Harlow and Lane, Antibodies: A Laboratory Manual (1989); Mol. Biology and Biotechnology: A Comprehensive Desk Reference (Myers ed., 1995); Huston et al., 1993, Cell Biophysics 22:189-224; Plückthun and Skerra, 1989, Meth. Enzymol. 178:497-515; and Day, Advanced Immunochemistry (2d ed. 1990). The antibodies provided herein can be of any class (e.g., IgG, IgE, IgM, IgD, and IgA) or any subclass (e.g., IgG1, IgG2, IgG3, IgG4, IgA1, and IgA2) of immunoglobulin molecule. Antibodies may be agonistic antibodies or antagonistic antibodies. Antibodies may be neither agonistic nor antagonistic.

An “antigen” is a structure to which an antibody can selectively bind. A target antigen may be a polypeptide, carbohydrate, nucleic acid, lipid, hapten, or other naturally occurring or synthetic compound. In some embodiments, the target antigen is a polypeptide. In certain embodiments, an antigen is associated with a cell, for example, is present on or in a cell.

An “intact” antibody is one comprising an antigen-binding site as well as a CL and at least heavy chain constant regions, CH1, CH2 and CH3. The constant regions may include human constant regions or amino acid sequence variants thereof. In certain embodiments, an intact antibody has one or more effector functions.

“Single-chain Fv” also abbreviated as “sFv” or “scFv” are antibody fragments that comprise the VH and VL antibody domains connected into a single polypeptide chain. Preferably, the scFv polypeptide further comprises a polypeptide linker between the VH and VL domains which enables the scFv to form the desired structure for antigen binding. For a review of the scFv, see Pluckthun in The Pharmacology of Monoclonal Antibodies, vol. 113, Rosenburg and Moore eds., Springer-Verlag, New York, pp. 269-315 (1994).

“Single domain antibody” or “sdAb” as used herein refers to a single monomeric variable antibody domain and which is capable of antigen binding. Single domain antibodies include V_{H}H domains as described herein. Examples of single domain antibodies include, but are not limited to, antibodies naturally devoid of light chains such as those from Camelidae species (e.g., llama), single domain antibodies derived from conventional 4-chain antibodies, engineered antibodies and single domain scaffolds other than those derived from antibodies. Single domain antibodies may be derived from any species including, but not limited to mouse, human, camel, llama, goat, rabbit, and bovine. For example, a single domain antibody can be derived from antibodies raised in Camelidae species, for example in camel, llama, fromedary, alpaca and guanaco, as described herein. Other species besides Camelidae may produce heavy chain antibodies naturally devoid of light chain; V_{H}Hs derived from such other species are within the scope of the disclosure. In some embodiments, the single domain antibody (e.g., V_{H}H) provided herein has a structure of FR1-CDR1-FR2-CDR2-FR3-CDR3-FR4. Single domain antibodies may be genetically fused or chemically conjugated to another molecule (e.g., an agent) as described herein. Single domain antibodies may be part of a bigger binding molecule (e.g., a multispecific antibody or a chimeric antigen receptor).

The term “binds”, “binding”, “specifically bind to” or is “specific for” refers to measurable and reproducible interactions such as binding between a target and an antibody, which is determinative of the presence of the target in the presence of a heterogeneous population of molecules including biological molecules. For example, an antibody that binds to or specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, e.g., by a radioimmunoassay (RIA). In certain embodiments, an antibody that specifically binds to a target has a dissociation constant (Kd) of ≤1 μM, ≤100 nM, ≤10 nM, ≤1 nM, or ≤0.1 nM. In certain embodiments, an antibody specifically binds to an epitope on a protein that is conserved among the protein from different species. In some embodiment, specific binding can include, but does not require exclusive binding. In another embodiment, the term “binds” or “binding” refer to an interaction between molecules including, for example, to form a complex.

As used herein, an “epitope” is a term in the art and refers to a localized region of an antigen to which a binding molecule (e.g., an antibody comprising a single chain antibody sequence) can specifically bind. An epitope can be a linear epitope or a conformational, non-linear, or discontinuous epitope. In the case of a polypeptide antigen, for example, an epitope can be contiguous amino acids of the polypeptide (a “linear” epitope) or an epitope can comprise amino acids from two or more non-contiguous regions of the polypeptide (a “conformational,” “non-linear” or “discontinuous” epitope). It will be appreciated by one of skill in the art that, in general, a linear epitope may or may not be dependent on secondary, tertiary, or quaternary structure. For example, in some embodiments, a binding molecule binds to a group of amino acids regardless of whether they are folded in a natural three dimensional protein structure. In other embodiments, a binding molecule requires amino acid residues making up the epitope to exhibit a particular conformation (e.g., bend, twist, turn or fold) in order to recognize and bind the epitope.

“Percent (%) amino acid sequence identity” and “homology” with respect to a peptide, polypeptide or antibody sequence is defined as the percentage of amino acid residues in a candidate sequence that is identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

The term “chimeric cytokine receptor” or CCR as used herein is a molecule which comprises a cytokine receptor endodomain transmembrane and a heterologous ligand-binding exodomain. The heterologous exodomain binds a ligand other than the cytokine for which the cytokine receptor from which the endodomain was derived is selective. In this way, it is possible to alter the ligand specificity of a cytokine receptor by grafting on a heterologous binding specificity.

In some embodiments, CCR comprising transmembrane domain comprises a Janus Kinase (JAK)-binding domain. In some embodiments, the JAK-binding domain is derived from a group consisting of EPOR, GHR, TPOR, or a variant thereof. In some embodiments, the JAK-binding domain comprises an amino acid sequence of SEQ ID NO: 7.

In some embodiments, CCR comprising an intracellular domain derived from a cytokine receptor or of the compositions, e.g., IL2Rα, IL7Rα, IL9R-1, IL9R-2, IL9R-3, IL12Rβ1, IL12Rβ2, IL15Rα, IL15Rβ-1, IL15Rβ-2, IL15Rβ-3, IL18Rα, IL18Rβ, IL21R-1, IL21R-2, IL21R-3, IL10R2, IL22R1, IL23R-1, IL23R-2, IL23R-3, IL27Rα, gp130, IL31RA, OSMRβ, IL36R, IL1RAcP, GM-CSFRα, GM-CSFRβ-1, GM-CSFRβ-2, a variant thereof, or combinations thereof. The chimeric cytokine receptor may further comprises an extracellular domain and a transmembrane domain.

In some embodiments, CCR comprises an tag comprising an amino acid sequence of SEQ ID NOs: 4, 5 or 158-160, or an amino acid sequence having at least 75%, 80%, 90%, 91%, 92%, 93%, 94%, 95%, 96%, 97%, 98%, or 99% identity to an amino acid sequence selected from SEQ ID NOs: 4, 5 or 158-160.

The term “functional exogenous receptor” as used herein, refers to an exogenous receptor (e.g., TCR such as a recombinant or engineered TCR, cTCR, TAC-like chimeric receptor, or CAR) that retains its biological activity after being introduced into an immune effector cell such as a T cell. The biological activity include but are not limited to the ability of the exogenous receptor in specifically binding to a molecule, properly transducing downstream signals, such as inducing cellular proliferation, cytokine production and/or performance of regulatory or cytolytic effector functions.

“Chimeric antigen receptor” or “CAR” as used herein refers to genetically engineered receptors, which can be used to graft one or more antigen specificity onto immune effector cells, such as T cells. Some CARs are also known as “artificial T-cell receptors,” “chimeric T cell receptors,” or “chimeric immune receptors.” A chimeric molecule that includes one or more antigen-binding portion (such as a single domain antibody or scFv) and a signaling domain, such as a signaling domain from a T cell receptor (e.g., CD3ζ). Typically, CARs are comprised of an antigen-binding moiety, a transmembrane domain and an intracellular domain. The intracellular domain typically includes a signaling chain having an immunoreceptor tyrosine-based activation motif (ITAM), such as CD3ζ or FcεRIγ. In some instances, the intracellular domain further includes the intracellular portion of at least one additional co-stimulatory domain, such as CD28, 4-1BB (CD137), ICOS, OX40 (CD134), CD27, and/or hematopoietic cell signal transducer (DAP10). In the context of the present application, the terms “cytoplasmic domain”, “intracellular domain” and “intracellular signaling domain” are interchangeable. In some embodiments, the CAR comprises an extracellular antigen binding domain specific for one or more antigens (such as tumor antigens), a transmembrane domain, and an intracellular signaling domain of a T cell and/or other receptors. In some embodiments, the CAR is single CAR. In some embodiments, the CAR is dual CAR. In some embodiments, the CAR is tandem CAR. In some embodiments, the CAR is split CAR. “CAR-T cell” refers to a T cell that expresses a CAR. The term “single CAR” as used herein is included as a chimeric molecule that includes a single antigen-binding portion (such as a single domain antibody or scFv) and a signaling domain, such as a signaling domain from a T cell receptor (e.g., CD3ζ). Typically, single CARs may comprise a monospecific antigen-binding moiety, a transmembrane domain, and an intracellular domain.

The term “tandem CAR” as used herein is included as a chimeric molecule that includes more than one antigen-binding portions (such as 2, 4, or 6 single domain antibodies or scFv) and a signaling domain, such as a signaling domain from a T cell receptor (e.g., CD3ζ). Typically, tandem CARs may comprise monospecific, bivalent antigen-binding moiety, e.g., two identical V_{H}H domains binding GPC3, or multi-specific, e.g., bispecific bivalent, antigen-binding moiety, e.g., two different V_{H}H domains binding GPC3 or one V_{H}H domain binding GPC3 and the other V_{H}H domain binding a molecule other than GPC3, a transmembrane domain, and an intracellular domain. In some embodiments, the tandem CAR of the present disclosure may include an extracellular antigen binding domain further comprising a second binding domain that binds to a second antigen, and the second antigen is different from the GPC3.

The term “dual CAR” as used herein is included as two separate CARs that may be bispecific, or two tandem CARs that each CAR may be monospecific or bispecific, or one single CAR and one tandem CAR that may be monospecific or bispecific. A dual CAR may have each of a first CAR and a second CAR having both co-stimulatory domain, such as CD28, 4-1BB (CD137), ICOS, OX40 (CD134), CD27, and DAP10, and a signaling domain, such as a signaling domain from a T cell receptor (e.g., CD3ζ). Dual CAR can be a combination of any two anti-GPC3 CARs, in which each of a first CAR and a second CAR may be a single CAR or a tandem CAR, i.e., single CAR/single CAR, single CAR/tandem CAR, or tandem CAR/tandem CAR. The levels of dual CAR T cell signalling may be regulated by manipulating the intracellular domains of each first and second CARs. For example, the intracellular domains of each of the first CAR and the second CAR may comprise a co-stimulatory domain, such as CD28, CD137 (4-1BB), ICOS, OX40 (CD134), CD27, and/or DAP10, and/or a signaling domain from a T cell receptor, such as a signaling domain from a T cell receptor (e.g., CD3ζ). For example, dual CAR of the present disclosure may include a first CAR and a second CAR each having an intracellular domain comprising a co-stimulatory domain and a signaling domain from a T cell receptor. Thus, when dual CAR bind antigens (e.g., bispecific), the T cell signals may be transmitted through two signaling domains from a T cell receptor.

The term “split CAR” as used herein is different from dual CAR, in split CAR, a first CAR may comprise a co-stimulatory domain but lack a signaling domain from a T cell receptor; and a second CAR may comprises signaling domain from a T cell receptor but lack co-stimulatory domain. In some embodiments, the split CAR system of the present disclosure may include a first intracellular signaling domain comprising a primary intracellular signaling domain of an immune effector cell and a second intracellular signaling domain comprising a co-stimulatory signaling domain.

The term “recombinant or engineered TCR” as used herein is included as a kind of functional exogenous receptor provided herein, and refers to peptide expressed into an immune cell. The functions of recombinant or engineered TCR may include for example redirecting immune activity of the immune cell against a desired type of cells, such as cancer and infected cells having specific markers at their surface. It can replace or be-co-expressed with the endogenous TCR. In some embodiments, such recombinant TCR are single-chain TCRs comprising an open reading frame where the variable Vα and Vβ domains are paired with a protein linker. This involves the molecular cloning of the TCR genes known to be specific for an antigen of choice. These chains are then introduced into T cells usually by means of a retroviral vector. Consequently, expression of the cloned TCRα and TCRβ genes endows the transduced T cell with a functional specificity determined by the pairing of these new genes. A component of a recombinant or engineered TCR is any functional subunit of a TCR, such as a recombined TCRα and TCRβ, which is encoded by an exogenous polynucleotide sequence introduced into the cell.

In some embodiments, the functional exogenous receptor provided herein is a chimeric TCR (cTCR), which has both antigen-binding and T-cell activating functions. For example, a cTCR can comprise: (a) an extracellular ligand binding domain comprising an antigen-binding fragment (e.g., sdAb, scFv) that specifically recognizes one or more epitopes of a tumor antigen (e.g., GPC3, CD19 or CLL1); (b) an optional linker; (c) an optional extracellular domain of a first TCR subunit (e.g., CD3ε) or a portion thereof, (d) a transmembrane domain comprising a transmembrane domain of a second TCR subunit (e.g., CD3ε); and (e) an intracellular signaling domain comprising an intracellular signaling domain of a third TCR subunit (e.g., CD3ε); wherein the first, second, and third TCR subunit are all selected from the group consisting of TCRα, TCRβ, TCRγ, TCRδ, CD3ε, CD3γ, and CD3δ. In some embodiments, the first, second, and third TCR subunits are the same (e.g., all CD3ε). In some embodiments, the first, second, and third TCR subunits are different. In some embodiments, the cTCR further comprises a hinge domain located between the C-terminus of the extracellular ligand binding domain and the N-terminus of the transmembrane domain. In some embodiments, the hinge domain is derived from CD8α. In some embodiments, the cTCR further comprises a signal peptide located at the N-terminus of the cTCR, such as a signal peptide derived from CD8α.

In some embodiments, the functional exogenous receptor is a T cell antigen coupler (TAC), e.g., comprising: (a) an extracellular ligand binding domain comprising an antigen-binding fragment (e.g., sdAb, scFv) that specifically recognizes one or more epitopes of a tumor antigen (e.g., GPC3, CD19 or CLL1); (b) an optional first linker; (c) an extracellular TCR binding domain that specifically recognizes the extracellular domain of a TCR subunit (e.g., CD3ε); (d) an optional second linker; (e) an optional extracellular domain of a first TCR co-receptor (e.g., CD4) or a portion thereof; (f) a transmembrane domain comprising a transmembrane domain of a second TCR co-receptor (e.g., CD4); and (g) an optional intracellular signaling domain comprising an intracellular signaling domain of a third TCR co-receptor (e.g., CD4); wherein the TCR subunit is selected from the group consisting of TCRα, TCRβ, TCRγ, TCRδ, CD3ε, CD3γ, and CD3δ; and wherein the first, second, and third TCR co-receptors are all selected from the group consisting of CD4, CD8, and CD28. In some embodiments, the first, second, and third TCR co-receptors are the same. In some embodiments, the first, second, and third TCR co-receptors are different. In some embodiments, the TAC further comprises a hinge domain located between the C-terminus of the extracellular ligand binding domain and the N-terminus of the transmembrane domain. In some embodiments, the hinge domain is derived from CD8α. In some embodiments, the TAC further comprises a signal peptide located at the N-terminus of the TAC, such as a signal peptide derived from CD8α. In some embodiments, the extracellular ligand binding domain is at N-terminal of the extracellular TCR binding domain. In some embodiments, the extracellular ligand binding domain is at C-terminal of the extracellular TCR binding domain.

In some embodiments, the functional exogenous receptor is a TAC-like chimeric receptor, e.g., comprising: (a) an extracellular ligand binding domain comprising an antigen-binding fragment (e.g., sdAb, scFv) that specifically recognizes one or more epitopes of a tumor antigen (e.g., GPC3); (b) an optional first linker; (c) an extracellular TCR binding domain that specifically recognizes the extracellular domain of a first TCR subunit (e.g., TCRα); (d) an optional second linker; (e) an optional extracellular domain of a second TCR subunit (e.g., CD3ε) or a portion thereof; (f) a transmembrane domain comprising a transmembrane domain of a third TCR subunit (e.g., CD3ε); and (g) an optional intracellular signaling domain comprising an intracellular signaling domain of a fourth TCR subunit (e.g., CD3ε); wherein the first, second, third, and fourth TCR subunits are all selected from the group consisting of TCRα, TCRβ, TCRγ, TCRδ, CD3ε, CD3γ, and CD3δ. In some embodiments, the second, third, and fourth TCR subunits are the same. In some embodiments, the first, second, third, and fourth TCR subunits are the same. In some embodiments, the first, second, third, and fourth TCR subunits are different. In some embodiments, the second, third, and fourth TCR subunits are the same, but different from the first TCR subunit. In some embodiments, the extracellular ligand binding domain is at N-terminal of the extracellular TCR binding domain. In some embodiments, the extracellular ligand binding domain is at C-terminal of the extracellular TCR binding domain. In some embodiments, the TAC-like chimeric receptor further comprises a hinge domain located between the C-terminus of the extracellular ligand binding domain and the N-terminus of the transmembrane domain. In some embodiments, the hinge domain is derived from CD8α. In some embodiments, the TAC-like chimeric receptor further comprises a signal peptide located at the N-terminus of the TAC-like chimeric receptor, such as a signal peptide derived from CD8α.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification. Also included within the definition are, for example, polypeptides containing one or more analogs of an amino acid, including but not limited to, unnatural amino acids, as well as other modifications known in the art. It is understood that, because the polypeptides of this disclosure may be based upon antibodies or other members of the immunoglobulin superfamily, in certain embodiments, a “polypeptide” can occur as a single chain or as two or more associated chains.

“Polynucleotide” or “nucleic acid,” as used interchangeably herein, refers to polymers of nucleotides of any length and includes DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. “Oligonucleotide,” as used herein, refers to short, generally single-stranded, synthetic polynucleotides that are generally, but not necessarily, fewer than about 200 nucleotides in length. The terms “oligonucleotide” and “polynucleotide” are not mutually exclusive. The description above for polynucleotides is equally and fully applicable to oligonucleotides. A cell that produces a binding molecule of the present disclosure may include a parent hybridoma cell, as well as bacterial and eukaryotic host cells into which nucleic acids encoding the antibodies have been introduced.

An “isolated nucleic acid” is a nucleic acid, for example, an RNA, DNA, or a mixed nucleic acids, which is substantially separated from other genome DNA sequences as well as proteins or complexes such as ribosomes and polymerases, which naturally accompany a native sequence. An “isolated” nucleic acid molecule is one which is separated from other nucleic acid molecules which are present in the natural source of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. In a specific embodiment, one or more nucleic acid molecules encoding a single chain antibody or an antibody as described herein are isolated or purified. The term embraces nucleic acid sequences that have been removed from their naturally occurring environment, and includes recombinant or cloned DNA isolates and chemically synthesized analogues or analogues biologically synthesized by heterologous systems. A substantially pure molecule may include isolated forms of the molecule. Specifically, an “isolated” nucleic acid molecule encoding a CAR or CAR armored CCR described herein is a nucleic acid molecule that is identified and separated from at least one contaminant nucleic acid molecule with which it is ordinarily associated in the environment in which it was produced.

The term “vector” refers to a substance that is used to carry or include a nucleic acid sequence, including for example, a nucleic acid sequence encoding a binding molecule (e.g., an antibody) as described herein, in order to introduce a nucleic acid sequence into a host cell. Vectors applicable for use include, for example, expression vectors, plasmids, phage vectors, viral vectors, episomes, and artificial chromosomes, which can include selection sequences or markers operable for stable integration into a host cell's chromosome. Additionally, the vectors can include one or more selectable marker genes and appropriate expression control sequences. Expression control sequences can include constitutive and inducible promoters, transcription enhancers, transcription terminators, and the like, which are well known in the art. When two or more nucleic acid molecules are to be co-expressed (e.g., both an antibody heavy and light chain or an antibody VH and VL), both nucleic acid molecules can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The introduction of nucleic acid molecules into a host cell can be confirmed using methods well known in the art. Such methods include, for example, nucleic acid analysis such as Northern blots or polymerase chain reaction (PCR) amplification of mRNA, immunoblotting for expression of gene products, or other suitable analytical methods to test the expression of an introduced nucleic acid sequence or its corresponding gene product. It is understood by those skilled in the art that expression levels can be optimized to obtain sufficient expression using methods well known in the art.

The term “host” as used herein refers to an animal, such as a mammal (e.g., a human).

The term “host cell” as used herein refers to a particular subject cell that may be transfected with a nucleic acid molecule and the progeny or potential progeny of such a cell. Progeny of such a cell may not be identical to the parent cell transfected with the nucleic acid molecule due to mutations or environmental influences that may occur in succeeding generations or integration of the nucleic acid molecule into the host cell genome.

As used herein, the term “autologous” is meant to refer to any material derived from the same individual to whom it is later to be re-introduced into the individual.

“Allogeneic” refers to a graft derived from a different individual of the same species.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The term “immune response” used here is included a response of a cell of the immune system, such as a B cell, T cell, or monocyte, to a stimulus. In some embodiments, the response is specific for a particular antigen (an “antigen-specific response”). In some embodiments, an immune response is a T cell response, such as a CD4⁺ response or a CD8⁺response. In another embodiment, the response is a B cell response, and results in the production of specific antibodies.

The term “pharmaceutically acceptable” as used herein means being approved by a regulatory agency of the Federal or a state government, or listed in United States Pharmacopeia, European Pharmacopeia, or other generally recognized Pharmacopeia for use in animals, and more particularly in humans. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Lippincott Williams & Wilkins: Philadelphia, P A, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, F L, 2009. In some embodiments, pharmaceutically acceptable excipients are nontoxic to the cell or mammal being exposed thereto at the dosages and concentrations employed. In some embodiments, a pharmaceutically acceptable excipient is an aqueous pH buffered solution.

“Carrier” or “Excipient” means a pharmaceutically-acceptable material, composition, or vehicle, such as a liquid or solid filler, diluent, solvent, or encapsulating material. Excipients include, for example, encapsulating materials or additives such as absorption accelerators, antioxidants, binders, buffers, carriers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof. The term “excipient” can also refer to a diluent, adjuvant (e.g., Freunds' adjuvant (complete or incomplete) or vehicle).

The term “effective amount” or “therapeutically effective amount” as used herein refers to the amount of engineered immune effector cells or a therapeutic molecule comprising an agent and the engineered immune effector cells or pharmaceutical composition provided herein which is sufficient to result in the desired outcome.

The terms “subject” and “patient” may be used interchangeably. As used herein, in certain embodiments, a subject is a mammal, such as a non-primate or a primate (e.g., human). In specific embodiments, the subject is a human. In one embodiment, the subject is a mammal, e.g., a human, diagnosed with a disease or disorder. In another embodiment, the subject is a mammal, e.g., a human, at risk of developing a disease or disorder.

“Administer” or “administration” refers to the act of injecting or otherwise physically delivering a substance as it exists outside the body into a patient, such as by mucosal, intradermal, intravenous, intramuscular delivery, and/or any other method of physical delivery described herein or known in the art.

As used herein, the terms “treat,” “treatment” and “treating” refer to the reduction or amelioration of the progression, severity, and/or duration of a disease or condition resulting from the administration of one or more therapies. Treating may be determined by assessing whether there has been a decrease, alleviation and/or mitigation of one or more symptoms associated with the underlying disorder such that an improvement is observed with the patient, despite that the patient may still be afflicted with the underlying disorder. The term “treating” includes both managing and ameliorating the disease.

The terms “manage”, “managing” and “management” refer to the beneficial effects that a subject derives from a therapy which does not necessarily result in a cure of the disease.

The terms “prevent”, “preventing” and “prevention” refer to reducing the likelihood of the onset (or recurrence) of a disease, disorder, condition, or associated symptom(s) (e.g., a cancer).

As used herein, “delaying” the development of cancer means to defer, hinder, slow, retard, stabilize, and/or postpone development of the disease. This delay can be of varying lengths of time, depending on the history of the disease and/or individual being treated. As is evident to one skilled in the art, a sufficient or significant delay can, in effect, encompass prevention, in that the individual does not develop the disease. A method that “delays” development of cancer is a method that reduces probability of disease development in a given time frame and/or reduces the extent of the disease in a given time frame, when compared to not using the method. Such comparisons are typically based on clinical studies, using a statistically significant number of individuals. Cancer development can be detectable using standard methods, including, but not limited to, computerized axial tomography (CAT Scan), Magnetic Resonance Imaging (MRI), abdominal ultrasound, clotting tests, arteriography, or biopsy. Development may also refer to cancer progression that may be initially undetectable and includes occurrence, recurrence, and onset.

The terms “about” and “approximately” mean within 20%, within 15%, within 10%, within 9%, within 8%, within 7%, within 6%, within 5%, within 4%, within 3%, within 2%, within 1%, or less of a given value or range.

As used in the present disclosure and claims, the singular forms “a”, “an” and “the” include plural forms unless the context clearly dictates otherwise.

It is understood that wherever embodiments are described herein with the term “comprising” otherwise analogous embodiments described in terms of “consisting of” and/or “consisting essentially of” are also provided. It is also understood that wherever embodiments are described herein with the phrase “consisting essentially of” otherwise analogous embodiments described in terms of “consisting of” are also provided.

The term “between” as used in a phrase as such “between A and B” or “between A-B” refers to a range including both A and B.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### 5.2. Engineered Immune Effector Cells

As shown in Section 6 below, introducing a chimeric cytokine receptor into immune effector cells expressing one or more functional exogenous receptor(s) can significantly improves properties/functions of the immune effector cells. In some embodiments, an immune effector cell expressing (i) a chimeric cytokine receptor comprising (a) a first extracellular antigen binding domain, wherein the first extracellular antigen binding domain is derived from NKG2D, truncated NKG2D, antibody or antigen binding fragment thereof targeting NKG2D ligands, TIGIT or SIRP-a, or a variant thereof, (b) a first transmembrane domain, and (c) a cytokine receptor intracellular domain; and (ii) optionally a functional exogenous receptor comprising (a) a second extracellular antigen binding domain, (b) a second transmembrane domain, and (c) an intracellular signaling domain.

Thus, in one aspect, provided herein are host cells (such as immune effector cells) comprising a chimeric cytokine receptor comprising (a) a first extracellular antigen binding domain, (b) a first transmembrane domain, and (c) a cytokine receptor intracellular domain. In some embodiments, the extracellular domain is derived from the extracellular domain of NKG2D or a variant thereof. In some embodiments, the extracellular antigen binding domain comprises the amino acid sequence of SEQ ID NO: 6. In some embodiments, the first extracellular antigen binding domain is derived from truncated NKG2D. In some embodiments, the first extracellular antigen binding domain is derived from truncated NKG2D (amino acid positions 89-216, SEQ ID NO: 157). In some embodiments, the first extracellular antigen binding domain is derived from truncated NKG2D (amino acid positions 98-216, SEQ ID NO: 156). In some embodiments, the extracellular antigen binding domain comprises an amino acid sequence having at least 75%, 80%, 85%, 90%, 91%, 92%, 93%, 94%, 95%, 96%, 97%, 98%, or 99% identity to SEQ ID NO: 6, 156 or 157.

In other embodiments, the extracellular domain comprises an antibody or antigen binding fragment thereof that binds to an NKG2D ligand. In some embodiments, the extracelluar domain comprises one or more scFv(s) that bind to an NKG2D ligand. In other embodiments, the extracellular domain comprises one or more sdAb(s) (such as one or more V_{H}H domains) that bind to an NKG2D ligand. The NKG2D ligand can be selected from (but not limited to) a group consisting of MICA/B, ULBP-1, ULBP-2,5,6 and ULBP-3.

In some embodiments, immune effector cell comprises the amino acid sequence of SEQ ID NOs: 29, 135, 141, 143 or 149 or an amino acid sequence having at least 75%, 80%, 85%, 90%, 91%, 92%, 93%, 94%, 95%, 96%, 97%, 98%, or 99% identity to SEQ ID NOs: 29, 135, 141,143 or 149. In some embodiments, immune effector cell comprising the amino acid sequence of SEQ ID NOs: 30-134, 136-140, 142, 144, 146 or 148, or an amino acid sequence having at least 75%, 80%, 85%, 90%, 91%, 92%, 93%, 94%, 95%, 96%, 97%, 98%, or 99% identity to SEQ ID NOs: 30-134, 136-140, 142, 144, 146 or 148. In some embodiments, immune effector cell comprises the amino acid sequence of SEQ ID NOs:150 or 151, or an amino acid sequence having at least 75%, 80%, 85%, 90%, 91%, 92%, 93%, 94%, 95%, 96%, 97%, 98%, or 99% identity to SEQ ID NOs:150 or 151.

NKG2D is a transmembrane protein belonging to the NKG2 family of C-type lectin-like receptors, and it is also referred to as KLRK1, CD314, D12S2489E, KLR, NKG2-D, NKG2 natural killer group 2D, killer cell lectin-like receptor K1, and killer cell lectin like receptor Kl. NKG2D is encoded by KLRK1 gene which is located in the NK-gene complex (NKC) situated on chromosome 6 in mice and chromosome 12 in humans. In humans, it is expressed by NK cells, γδ T cells and CD8⁺ αβ T cells. NKG2D recognizes induced-self proteins from MIC and RAET1/ULBP families which appear on the surface of stressed, malignant transformed, and infected cells. NKG2D ligands are induced-self proteins which are completely absent or present only at low levels on surface of normal cells, but they are overexpressed by infected, transformed, senescent and stressed cells. NKG2D sequences are known in the art (see, e.g., OMIM: 611817, HomoloGene: 136440, GeneCards: KLRK1).

In some embodiments, the first extracellular antigen binding domain is derived from TIGIT. In some embodiments, the first extracellular antigen binding domain comprises the amino acid sequence of SEQ ID NO: 145. In some embodiments, the first extracellular antigen binding domain is derived from SIRP-α. In some embodiments, the first extracellular antigen binding domain comprises the amino acid sequence of SEQ ID NO: 147.

In some embodiments, the host cells express one or more functional exogenous receptor(s). The functional exogenous receptors can be, for example, chimeric antigen receptor (CAR), engineered T cell receptor (TCR), chimeric TCR (cTCR), and T cell antigen coupler (TAC)-like chimeric receptor. In some embodiments, the functional exogenous receptor is a CAR. In some embodiments, the functional exogenous receptor is a TCR. In some embodiments, the functional exogenous receptor is cTCR. In yet other embodiments, the functional exogenous receptor is a TAC. Any immune effector cell that can perform immune effector functions may be used in the present disclosure, including but not limited to, peripheral blood mononuclear cells (PBMC), natural killer (NK) cells, monocytes, cytotoxic T cells, neutrophils, and eosinophils.

In some embodiments, provided herein are host cells, such as immune effector cells, comprising any one of the polypeptides, polynucleotides, or vectors described herein, for example, as provided in the following sections.

### 5.2.1. Chimeric Cytokine Receptor

In one aspect, provided herein is a chimeric cytokine receptor comprising an intracellular domain derived from one or more cytokine receptor(s), i.e., cytokine receptor intracellular domain. The chimeric cytokine receptor provided herein also comprises an extracellular domain and a transmembrane domain.

### Cytokine Receptor Intracellular Domain

In some embodiments, the cytokine receptors employed in the present chimeric cytokine receptors are selected from a group consisting IL2Rα, IL7Rα, IL9R-1, IL9R-2, IL9R-3, IL12Rβ1, IL12Rβ2, IL15Rα, IL15Rβ-1, IL15Rβ-2, IL15Rβ-3, IL18Rα, IL18Rβ, IL21R-1, IL21R-2, IL21R-3, IL10R2, IL22R1, IL23R-1, IL23R-2, IL23R-3, IL27Rα, gp130, IL31RA, OSMRβ, IL36R, IL1RAcP, GM-CSFRα, GM-CSFRβ-1, GM-CSFRβ-2, a variant thereof, and combinations thereof.

In some embodiments, the chimeric cytokine receptor provided herein comprises an intracellular domain comprising a region derived from IL2Rα. In some embodiments, the chimeric cytokine receptor provided herein comprises an intracellular domain comprising a region derived from IL7Rα. In some embodiments, the chimeric cytokine receptor provided herein comprises an intracellular domain comprising a region derived from IL9R-1. In some embodiments, the chimeric cytokine receptor provided herein comprises an intracellular domain comprising a region derived from IL9R-2. In some embodiments, the chimeric cytokine receptor provided herein comprises an intracellular domain comprising a region derived from IL9R-3. In some embodiments, the chimeric cytokine receptor provided herein comprises an intracellular domain comprising a region derived from IL12Rβ1. In some embodiments, the chimeric cytokine receptor provided herein comprises an intracellular domain comprising a region derived from IL12Rβ2. In some embodiments, the chimeric cytokine receptor provided herein comprises an intracellular domain comprising a region derived from IL15Rα. In some embodiments, the chimeric cytokine receptor provided herein comprises an intracellular domain comprising a region derived from IL15Rβ-1. In some embodiments, the chimeric cytokine receptor provided herein comprises an intracellular domain comprising a region derived from IL15Rβ-2. In some embodiments, the chimeric cytokine receptor provided herein comprises an intracellular domain comprising a region derived from IL15Rβ-3. In some embodiments, the chimeric cytokine receptor provided herein comprises an intracellular domain comprising a region derived from IL-18Rα. In some embodiments, the chimeric cytokine receptor provided herein comprises an intracellular domain comprising a region derived from IL18Rβ. In some embodiments, the chimeric cytokine receptor provided herein comprises an intracellular domain comprising a region derived from IL21R-1. In some embodiments, the chimeric cytokine receptor provided herein comprises an intracellular domain comprising a region derived from IL21R-2. In some embodiments, the chimeric cytokine receptor provided herein comprises an intracellular domain comprising a region derived from IL21R-3. In some embodiments, the chimeric cytokine receptor provided herein comprises an intracellular domain comprising a region derived from IL-10R2. In some embodiments, the chimeric cytokine receptor provided herein comprises an intracellular domain comprising a region derived from IL22R1. In some embodiments, the chimeric cytokine receptor provided herein comprises an intracellular domain comprising a region derived from IL23R-1. In some embodiments, the chimeric cytokine receptor provided herein comprises an intracellular domain comprising a region derived from IL23R-2. In some embodiments, the chimeric cytokine receptor provided herein comprises an intracellular domain comprising a region derived from IL23R-3. In some embodiments, the chimeric cytokine receptor provided herein comprises an intracellular domain comprising a region derived from IL-27Rα. In some embodiments, the chimeric cytokine receptor provided herein comprises an intracellular domain comprising a region derived from gp130. In some embodiments, the chimeric cytokine receptor provided herein comprises an intracellular domain comprising a region derived from IL3IRA. In some embodiments, the chimeric cytokine receptor provided herein comprises an intracellular domain comprising a region derived from OSMRβ. In some embodiments, the chimeric cytokine receptor provided herein comprises an intracellular domain comprising a region derived from IL36R. In some embodiments, the chimeric cytokine receptor provided herein comprises an intracellular domain comprising a region derived from IL1RAcP. In some embodiments, the chimeric cytokine receptor provided herein comprises an intracellular domain comprising a region derived from GM-CSFRα. In some embodiments, the chimeric cytokine receptor provided herein comprises an intracellular domain comprising a region derived from GM-CSFRβ-1. In some embodiments, the chimeric cytokine receptor provided herein comprises an intracellular domain comprising a region derived from GM-CSFRβ-2.

In some embodiments, the intracellular domain of the chimeric cytokine receptor provided herein comprises sequences from two or more (such as 2, 3, 4, 5 or more) of IL2Rα, IL7Rα, IL9R-1, IL9R-2, IL9R-3, IL12Rβ1, IL12Rβ2, IL15Rα, IL15Rβ-1, IL15Rβ-2, IL15Rβ-3, IL18Rα, IL18Rβ, IL21R-1, IL21R-2, IL21R-3, IL10R2, IL22R1, IL23R-1, IL23R-2, IL23R-3, IL27Rα, gp130, IL31RA, OSMRβ, TL36R, IL1RAcP, GM-CSFRα, GM-CSFRβ-1, GM-CSFRβ-2, a variant thereof, or combinations thereof.

For example, in some embodiments, the intracellular domain of the chimeric cytokine receptor provided herein comprises a region derived from IL2Ra and a region derived from IL7Rα, IL2Rα, IL9R-1, IL9R-2, IL9R-3, IL12Rβ1, IL12Rβ2, IL15Rα, IL15Rβ-1, IL15Rβ-2, IL15Rβ-3, IL18Rα, IL18Rβ, IL21R-1, IL21R-2, IL21R-3, IL10R2, IL22R1, IL23R-1, IL23R-2, IL23R-3, IL27Rα, gp130, IL31RA, OSMRβ, IL36R, IL1RAcP, GM-CSFRα, GM-CSFRβ-1, GM-CSFRβ-2, a variant thereof, or combinations thereof. For example, in some embodiments, the intracellular domain of the chimeric cytokine receptor provided herein comprises a region derived from IL7Ra and a region derived from IL9R-1, IL9R-2, IL9R-3, IL12RP 1, IL12Rβ2, IL15Rα, IL15Rβ-1, IL15Rβ-2, IL15Rβ-3, IL18Rα, IL18Rβ, IL21R-1, IL21R-2, IL21R-3, IL10R2, IL22R1, IL23R-1, IL23R-2, IL23R-3, IL27Rα, gp130, IL31RA, OSMRβ, IL36R, IL1RAcP, GM-CSFRα, GM-CSFRβ-1, GM-CSFRβ-2, a variant thereof, or combinations thereof. For example, in some embodiments, the intracellular domain of the chimeric cytokine receptor provided herein comprises a region derived from IL9R-1 and a region derived from IL9R-2, IL9R-3, IL12Rβ1, IL12Rβ2, IL15Rα, IL15Rβ-1, IL15Rβ-2, IL15Rβ-3, IL18Rα, IL18Rβ, IL21R-1, IL21R-2, IL21R-3, IL10R2, IL22R1, IL23R-1, IL23R-2, IL23R-3, IL27Rα, gp130, IL31RA, OSMRβ, IL36R, IL1RAcP, GM-CSFRα, GM-CSFRβ-1, GM-CSFRβ-2, a variant thereof, or combinations thereof. For example, in some embodiments, the intracellular domain of the chimeric cytokine receptor provided herein comprises a region derived from IL9R-2 and a region derived from IL9R-3, IL12Rβ1, IL12Rβ2, IL15Rα, IL15Rβ-1, IL15Rβ-2, IL15Rβ-3, IL18Rα, IL18Rβ, IL21R-1, IL21R-2, IL21R-3, IL10R2, IL22R1, IL23R-1, IL23R-2, IL23R-3, IL27Rα, gp130, IL31RA, OSMRβ, IL36R, IL1RAcP, GM-CSFRα, GM-CSFRβ-1, GM-CSFRβ-2, a variant thereof, or combinations thereof. For example, in some embodiments, the intracellular domain of the chimeric cytokine receptor provided herein comprises a region derived from IL9R-3 and a region derived from IL12Rβ1, IL12Rβ2, IL15Rα, IL15Rβ-1, IL15Rβ-2, IL15Rβ-3, IL18Rα, IL18Rβ, IL21R-1, IL21R-2, IL21R-3, IL10R2, IL22R1, IL23R-1, IL23R-2, IL23R-3, IL27Rα, gp130, IL31RA, OSMRβ, IL36R, IL1RAcP, GM-CSFRα, GM-CSFRβ-1, GM-CSFRβ-2, a variant thereof, or combinations thereof. For example, in some embodiments, the intracellular domain of the chimeric cytokine receptor provided herein comprises a region derived from IL12Rβ1 and a region derived from IL12Rβ2, IL15Rα, IL15Rβ-1, IL15Rβ-2, IL15Rβ-3, IL18Rα, IL18Rβ, IL21R-1, IL21R-2, IL21R-3, IL10R2, IL22R1, IL23R-1, IL23R-2, IL23R-3, IL27Rα, gp130, IL31RA, OSMRβ, IL36R, IL1RAcP, GM-CSFRα, GM-CSFRβ-1, GM-CSFRβ-2, a variant thereof, or combinations thereof. For example, in some embodiments, the intracellular domain of the chimeric cytokine receptor provided herein comprises a region derived from IL12Rβ2 and a region derived from IL15Rα, IL15Rβ-1, IL15Rβ-2, IL15Rβ-3, IL18Rα, IL18Rβ, IL21R-1, IL21R-2, IL21R-3, IL10R2, IL22R1, IL23R-1, IL23R-2, IL23R-3, IL27Rα, gp130, IL31RA, OSMRβ, IL36R, IL1RAcP, GM-CSFRα, GM-CSFRβ-1, GM-CSFRβ-2, or a variant thereof, or combinations thereof. For example, in some embodiments, the intracellular domain of the chimeric cytokine receptor provided herein comprises a region derived from IL15Ra and a region derived from IL15Rβ-1, IL15Rβ-2, IL15Rβ-3, IL18Rα, IL18Rβ, IL21R-1, IL21R-2, IL21R-3, IL10R2, IL22R1, IL23R-1, IL23R-2, IL23R-3, IL27Rα, gp130, IL31RA, OSMRβ, TL36R, IL1RAcP, GM-CSFRα, GM-CSFRβ-1, GM-CSFRβ-2, a variant thereof, or combinations thereof. For example, in some embodiments, the intracellular domain of the chimeric cytokine receptor provided herein comprises a region derived from IL15Rβ-1 and a region derived from IL15Rβ-2, IL15Rβ-3, IL18Rα, IL18Rβ, IL21R-1, IL21R-2, IL21R-3, IL10R2, IL22R1, IL23R-1, IL23R-2, IL23R-3, IL27Rα, gp130, IL31RA, OSMRβ, IL36R, IL1RAcP, GM-CSFRα, GM-CSFRβ-1, GM-CSFRβ-2, a variant thereof, or combinations thereof. For example, in some embodiments, the intracellular domain of the chimeric cytokine receptor provided herein comprises a region derived from IL15Rβ-2 and a region derived from IL15Rβ-3, IL18Rα, IL18Rβ, IL21R-1, IL21R-2, IL21R-3, IL10R2, IL22R1, IL23R-1, IL23R-2, IL23R-3, IL27Rα, gp130, IL31RA, OSMRβ, IL36R, IL1RAcP, GM-CSFRα, GM-CSFRβ-1, GM-CSFRβ-2, a variant thereof, or combinations thereof. For example, in some embodiments, the intracellular domain of the chimeric cytokine receptor provided herein comprises a region derived from IL15Rβ-3 and a region derived from IL18Rα, IL18Rβ, IL21R-1, IL21R-2, IL21R-3, IL10R2, IL22R1, IL23R-1, IL23R-2, IL23R-3, IL27Rα, gp130, IL31RA, OSMRβ, IL36R, IL1RAcP, GM-CSFRα, GM-CSFRβ-1, GM-CSFRβ-2, a variant thereof, or combinations thereof. For example, in some embodiments, the intracellular domain of the chimeric cytokine receptor provided herein comprises a region derived from IL18Ra and a region derived from IL18R, IL18Rβ, IL21R-1, IL21R-2, IL21R-3, IL10R2, IL22R1, IL23R-1, IL23R-2, IL23R-3, IL27Rα, gp130, IL31RA, OSMRβ, IL36R, IL1RAcP, GM-CSFRα, GM-CSFRβ-1, GM-CSFRβ-2, a variant thereof, or combinations thereof. For example, in some embodiments, the intracellular domain of the chimeric cytokine receptor provided herein comprises a region derived from IL18RP and a region derived from IL21R-1 IL21R-2, IL21R-3, IL10R2, IL22R1, IL23R-1, IL23R-2, IL23R-3, IL27Rα, gp130, IL31RA, OSMRβ, IL36R, IL1RAcP, GM-CSFRα, GM-CSFRβ-1, GM-CSFRβ-2, a variant thereof, or combinations thereof. For example, in some embodiments, the intracellular domain of the chimeric cytokine receptor provided herein comprises a region derived from IL21R-1 and a region derived from IL21R-2, IL21R-3, IL10R2, IL22R1, IL23R-1, IL23R-2, IL23R-3, IL27Rα, gp130, IL31RA, OSMRβ, IL36R, IL1RAcP, GM-CSFRα, GM-CSFRβ-1, GM-CSFRβ-2, a variant thereof, or combinations thereof. For example, in some embodiments, the intracellular domain of the chimeric cytokine receptor provided herein comprises a region derived from IL21R-2 and a region derived from, IL21R-3, IL10R2, IL22R1, IL23R-1, IL23R-2, IL23R-3, IL27Rα, gp130, IL31RA, OSMRβ, IL36R, IL1RAcP, GM-CSFRα, GM-CSFRβ-1, GM-CSFRβ-2, a variant thereof, or combinations thereof. For example, in some embodiments, the intracellular domain of the chimeric cytokine receptor provided herein comprises a region derived from IL21R-3 and a region derived from IL10R2, IL22R1, IL23R-1, IL23R-2, IL23R-3, IL27Rα, gp130, IL31RA, OSMRβ, IL36R, IL1RAcP, GM-CSFRα, GM-CSFRβ-1, GM-CSFRβ-2, a variant thereof, or combinations thereof. For example, in some embodiments, the intracellular domain of the chimeric cytokine receptor provided herein comprises a region derived from IL10R2 and a region derived from IL22R1, IL23R-1, IL23R-2, IL23R-3, IL27Rα, gp130, IL31RA, OSMRβ, IL36R, IL1RAcP, GM-CSFRα, GM-CSFRβ-1, GM-CSFRβ-2, a variant thereof, or combinations thereof. For example, in some embodiments, the intracellular domain of the chimeric cytokine receptor provided herein comprises a region derived from IL22R1 and a region derived from IL23R-1, IL23R-2, IL23R-3, IL27Rα, gp130, IL31RA, OSMRβ, IL36R, IL1RAcP, GM-CSFRα, GM-CSFRβ-1, GM-CSFRβ-2, a variant thereof, or combinations thereof. For example, in some embodiments, the intracellular domain of the chimeric cytokine receptor provided herein comprises a region derived from IL22R1 and a region derived from GM-CSFRβ-1, GM-CSFRβ-2, a variant thereof, or combinations thereof. For example, in some embodiments, the intracellular domain of the chimeric cytokine receptor provided herein comprises a region derived from IL23R-1 and a region derived from IL23R-2, IL23R-3, IL27Rα, gp130, IL31RA, OSMRβ, IL36R, IL1RAcP, GM-CSFRα, GM-CSFRβ-1, GM-CSFRβ-2, a variant thereof, or combinations thereof. For example, in some embodiments, the intracellular domain of the chimeric cytokine receptor provided herein comprises a region derived from IL23R-2 and a region derived from IL23R-3, IL27Rα, gp130, IL31RA, OSMRβ, IL36R, IL1RAcP, GM-CSFRα, GM-CSFRβ-1, GM-CSFRβ-2, a variant thereof, or combinations thereof. For example, in some embodiments, the intracellular domain of the chimeric cytokine receptor provided herein comprises a region derived from IL23R-3 and a region derived from IL27Rα, gp130, IL31RA, OSMRβ, IL36R, IL1RAcP, GM-CSFRα, GM-CSFRβ-1, GM-CSFRβ-2, a variant thereof, or combinations thereof. For example, in some embodiments, the intracellular domain of the chimeric cytokine receptor provided herein comprises a region derived from IL27Ra and a region derived from gp130, IL31RA, OSMRβ, IL36R, IL1RAcP, GM-CSFRα, GM-CSFRβ-1, GM-CSFRβ-2, a variant thereof, or combinations thereof. For example, in some embodiments, the intracellular domain of the chimeric cytokine receptor provided herein comprises a region derived from gp130 and a region derived from IL31RA, OSMRβ, IL36R, IL1RAcP, GM-CSFRα, GM-CSFRβ-1, GM-CSFRβ-2, a variant thereof, or combinations thereof. For example, in some embodiments, the intracellular domain of the chimeric cytokine receptor provided herein comprises a region derived from IL3IRA and a region derived from OSMRβ, IL36R, IL1RAcP, GM-CSFRα, GM-CSFRβ-1, GM-CSFRβ-2, a variant thereof, or combinations thereof. For example, in some embodiments, the intracellular domain of the chimeric cytokine receptor provided herein comprises a region derived from OSMRβ and a region derived from IL36R, IL1RAcP, GM-CSFRα, GM-CSFRβ-1, GM-CSFRβ-2, a variant thereof, or combinations thereof. For example, in some embodiments, the intracellular domain of the chimeric cytokine receptor provided herein comprises a region derived from IL36R and a region derived from IL1RAcP, GM-CSFRα, GM-CSFRβ-1, GM-CSFRβ-2, a variant thereof, or combinations thereof. For example, in some embodiments, the intracellular domain of the chimeric cytokine receptor provided herein comprises a region derived from IL1RAcP and a region derived from GM-CSFRα, GM-CSFRβ-1, GM-CSFRβ-2, GM-CSFRβ-1, GM-CSFRβ-2, a variant thereof, or combinations thereof. In some embodiments, the intracellular domain of the chimeric cytokine receptor provided herein comprises a region derived from GM-CSFRβ-1 and a region derived from GM-CSFRβ-2, or a variant thereof.

In some embodiments, the intracellular domain of the chimeric cytokine receptor provided herein comprises a region derived from IL-7Rα, IL12Rβ1, IL12Rβ2, IL15Rα, IL15Rβ-1, IL15Rβ-2, IL15Rβ-3, IL-18Rα, IL18Rβ, IL21R-1, IL21R-2, IL21R-3, IL23R-1, IL23R-2, IL23R-3, GM-CSFRα, GM-CSFRβ-1, GM-CSFRβ-2, IL9R-1, IL9R-2, IL9R-3, IL-7Rα-IL12Rβ1, IL-7Rα-IL15Rα, IL-7Rα-IL15Rβ-1, IL-7Rα-IL15Rβ-2, IL-7Rα-IL21R-1, IL-7Rα-IL21R-2, IL-7Rα-IL21R-3, IL-7Rα-IL23R-2, IL-7Rα-GMCSFRα, IL-7Rα-GM-CSFRβ-1, IL-7Rα-GM-CSFRβ-2, IL12Rβ1-IL15Rα, IL12Rβ1-IL15Rβ-1, IL12Rβ1-IL15Rβ-2, IL12Rβ1-IL-21R-1, IL12Rβ1-IL-21R-2, IL12Rβ1-IL-21R-3, IL12Rβ1-IL-23R-2, IL12Rβ1-GM-CSFRα, IL12Rβ1-GM-CSFRβ-1, IL12Rβ1-GM-CSFRβ-2, IL12Rβ2-IL15Rα, IL12Rβ2-IL15Rβ-1, IL12Rβ2-IL15Rβ-2, IL12Rβ2-IL-21R-1, IL12Rβ2-IL-21R-2, IL12Rβ2-IL-21R-3, IL12Rβ2-IL-23R-2, IL12Rβ2-GM-CSFRα, IL12Rβ2-GM-CSFRβ-1, IL12Rβ2-GM-CSFRβ-2, IL15Rα-IL-21R-1, IL15Rα-IL-21R-2, IL15Rα-IL-21R-3, IL15Rα-IL-23R-2, IL15Rα-GM-CSFRα, IL15Rα-GM-CSFRβ-1, IL15Rα-GM-CSFRβ-2, IL15Rβ-1-IL-21R-1, L15Rβ-1-IL-21R-2, IL15Rβ-1-IL-21R-3, IL15Rβ-1-IL-23R-2, IL15Rβ-1-GM-CSFRα, IL15Rβ-1-GM-CSFRβ-1, IL15Rβ-1-GM-CSFRβ-2, IL15Rβ-2-IL-21R-1, IL15Rβ-2-IL-21R-2, IL15Rβ-2-IL-21R-3, L15Rβ-2-IL-23R-2, IL15Rβ-2-GM-CSFRα, IL15Rβ-2-GM-CSFRβ-1, IL15Rβ-2-GM-CSFRβ-2, IL-21R-1-IL-23R-2, IL-21R-1-GM-CSFRα, IL-21R-1-GM-CSFRβ-1, IL-21R-1-GM-CSFRβ-2, IL-21R-2-IL-23R-2, IL-21R-2-GM-CSFRα, IL-21R-2-GM-CSFRβ-1, IL-21R-2-GM-CSFRβ-2, IL-21R-3-IL-23R-2, IL-21R-3-GM-CSFRα, IL-21R-3-GM-CSFRβ-1, IL-21R-3-GM-CSFRβ-2, IL-23R-2-GM-CSFRα, IL-23R-2-GM-CSFRβ-1, IL-23R-2-GM-CSFRβ-2, IL-7Rα-IL-9R-2, IL12Rβ1-IL-9R-2, IL12Rβ2-IL-9R-2, IL15Rα-IL-9R-2, IL15Rβ-1-IL-9R-2, IL15Rβ-2-IL-9R-2, IL-21R-1-IL-9R-2, IL-21R-2-IL-9R-2, IL-21R-3-IL-9R-2, IL-23R-2-IL-9R-2, GM-CSFRα-IL-9R-2, GM-CSFRβ-1-IL-9R-2, GM-CSFRβ-2-IL-9R-2, IL-7Rα-IL-12Rβ2, or a variant thereof.

When the intracellular domain of the chimeric cytokine receptor provided herein comprises sequences from two or more cytokine receptors, these sequences can be in any order. In some embodiments, the cytokine receptor intracellular domain comprises a region having an amino acid sequence selected from SEQ ID NOs: 8-28, or an amino acid sequence having at least 75%, 80%, 90%, 91%, 92%, 93%, 94%, 95%, 96%, 97%, 98%, or 99% identity to an amino acid sequence selected from SEQ ID NOs: 8-28. In a specific embodiment, the cytokine receptor intracellular domain comprises one or more the amino acid sequences of SEQ ID NO: 8, 10, 14-17 or 20-28.

### Transmembrane Domain

The chimeric cytokine receptors of the present disclosure comprise a transmembrane domain that can be directly or indirectly fused to the extracellular antigen binding domain. The transmembrane domain may be derived either from a natural or from a synthetic source. As used herein, a “transmembrane domain” refers to any protein structure that is thermodynamically stable in a cell membrane, e.g., a eukaryotic cell membrane. Transmembrane domains compatible for use in the chimeric cytokine receptors described herein may be obtained from a naturally occurring protein. Alternatively, it can be a synthetic, non-naturally occurring protein segment, e.g., a hydrophobic protein segment that is thermodynamically stable in a cell membrane.

Transmembrane domains are classified based on the three dimensional structure of the transmembrane domain. For example, transmembrane domains may form an alpha helix, a complex of more than one alpha helix, a beta-barrel, or any other stable structure capable of spanning the phospholipid bilayer of a cell. Furthermore, transmembrane domains may also or alternatively be classified based on the transmembrane domain topology, including the number of passes that the transmembrane domain makes across the membrane and the orientation of the protein. For example, single-pass membrane proteins cross the cell membrane once, and multi-pass membrane proteins cross the cell membrane at least twice (e.g., 2, 3, 4, 5, 6, 7 or more times). Membrane proteins may be defined as Type I, Type II or Type III depending upon the topology of their termini and membrane-passing segment(s) relative to the inside and outside of the cell. Type I membrane proteins have a single membrane-spanning region and are oriented such that the N-terminus of the protein is present on the extracellular side of the lipid bilayer of the cell and the C-terminus of the protein is present on the cytoplasmic side. Type II membrane proteins also have a single membrane-spanning region but are oriented such that the C-terminus of the protein is present on the extracellular side of the lipid bilayer of the cell and the N-terminus of the protein is present on the cytoplasmic side. Type III membrane proteins have multiple membrane-spanning segments and may be further sub-classified based on the number of transmembrane segments and the location of N- and C-termini.

In some embodiments, the transmembrane domain of the chimeric cytokine receptor described herein is derived from a Type I single-pass membrane protein. In some embodiments, transmembrane domains from multi-pass membrane proteins may also be compatible for use in the chimeric cytokine receptors described herein. Multi-pass membrane proteins may comprise a complex (at least 2, 3, 4, 5, 6, 7 or more) alpha helices or a beta sheet structure. In some embodiments, the N-terminus and the C-terminus of a multi-pass membrane protein are present on opposing sides of the lipid bilayer, e.g., the N-terminus of the protein is present on the cytoplasmic side of the lipid bilayer and the C-terminus of the protein is present on the extracellular side.

Transmembrane domains for use in the chimeric cytokine receptors described herein can also comprise at least a portion of a synthetic, non-naturally occurring protein segment. In some embodiments, the transmembrane domain is a synthetic, non-naturally occurring alpha helix or beta sheet. In some embodiments, the protein segment is at least approximately 20 amino acids, e.g., at least 18, 19, 20, 21, 22, 23, 24, 25, 26, 27, 28, 29, 30, or more amino acids. Examples of synthetic transmembrane domains are known in the art, for example in U.S. Pat. No. 7,052,906 and PCT Publication No. WO 2000/032776, the relevant disclosures of which are incorporated by reference herein.

The transmembrane domain provided herein may comprise a transmembrane region and a cytoplasmic region located at the C-terminal side of the transmembrane domain. The cytoplasmic region of the transmembrane domain may comprise three or more amino acids and, in some embodiments, helps to orient the transmembrane domain in the lipid bilayer. In some embodiments, one or more cysteine residues are present in the transmembrane region of the transmembrane domain. In some embodiments, one or more cysteine residues are present in the cytoplasmic region of the transmembrane domain. In some embodiments, the cytoplasmic region of the transmembrane domain comprises positively charged amino acids. In some embodiments, the cytoplasmic region of the transmembrane domain comprises the amino acids arginine, serine, and lysine.

In some embodiments, the transmembrane region of the transmembrane domain comprises hydrophobic amino acid residues. In some embodiments, the transmembrane domain of the chimeric cytokine receptor provided herein comprises an artificial hydrophobic sequence. For example, a triplet of phenylalanine, tryptophan and valine may be present at the C terminus of the transmembrane domain. In some embodiments, the transmembrane region comprises mostly hydrophobic amino acid residues, such as alanine, leucine, isoleucine, methionine, phenylalanine, tryptophan, or valine. In some embodiments, the transmembrane region is hydrophobic. In some embodiments, the transmembrane region comprises a poly-leucine-alanine sequence. The hydropathy, or hydrophobic or hydrophilic characteristics of a protein or protein segment, can be assessed by any method known in the art, for example the Kyte and Doolittle hydropathy analysis.

In some embodiments, the transmembrane domain of the chimeric cytokine receptor comprises a transmembrane domain chosen from the transmembrane domain of an alpha, beta or zeta chain of a T-cell receptor, CD28, CD3 epsilon, CD45, CD4, CD5, CD8, CD9, CD16, CD22, CD33, CD3γ, CD64, CD80, CD86, CD134, CD137, CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL-2R beta, IL-2R gamma, IL-7R a, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CDIOO (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and/or NKG2C.

In some embodiments, the transmembrane domain of the present chimeric cytokine receptor comprises a Janus Kinase (JAK)-binding domain. In some embodiments, the JAK-binding domain is derived from a group consisting of EPOR, GHR, TPOR, or a variant thereof. In some embodiments, the JAK-binding domain is derived from TPOR. In some embodiments, the JAK-binding domain comprises the amino acid sequence of SEQ ID NO: 7. In some embodiments, the JAK-binding domain is derived from EPOR. In some embodiments, the JAK-binding domain is derived from GHR. In some embodiments, the transmembrane domain of the present chimeric cytokine receptor comprises a JAK-binding domain in addition to a transmembrane domain described above.

### Extracellular Domain

In some embodiments, the extracellular domain of the present chimeric cytokine receptor binds to an antigen expressed on the surface of a target cell, such as a tumor cell or an infected cell.

The extracellular domain of the chimeric cytokine receptors described herein comprises one or more antigen binding domains. The extracellular domain of the chimeric cytokine receptors provided herein can be in any format as long as the binding of the extracellular domain to its target activates downstream intracellular cytokine signals, e.g., triggering the dimerization of the chimeric cytokine receptors. In some embodiments, the extracellular domain is derived from a naturally occurring receptor (e.g., an ECD of a receptor). In other embodiments, the extracellular domain is not derived from a naturally occurring receptor. In some embodiments, the first extracellular antigen binding domain is derived from NKG2D or truncated NKG2D (such as the ECD of NKG2D or truncated NKG2D), or a variant thereof. In some embodiments, the first extracellular antigen binding domain comprises the amino acid sequence of SEQ ID NOs: 6, 156 or 157, or an amino acid sequence having at least 75%, 80%, 85%, 90%, 91%, 92%, 93%, 94%, 95%, 96%, 97%, 98%, or 99% identity to SEQ ID NOs: 6, 156 or 157. In some embodiments, the first extracellular antigen binding domain is derived from truncated NKG2D (amino acid positions 89-216, SEQ ID NO: 157). In some embodiments, the first extracellular antigen binding domain is derived from truncated NKG2D (amino acid positions 98-216, SEQ ID NO: 156). In some embodiments, the first extracellular antigen binding domain comprises the amino acid sequence of SEQ ID NO: 6. In some embodiments, the first extracellular antigen binding domain is derived from TIGIT (such as the ECD of TIGIT). In some embodiments, the first extracellular antigen binding domain comprises the amino acid sequence of SEQ ID NO: 145. In some embodiments, the first extracellular antigen binding domain is derived from SIRP-α (such as the ECD of SIRP-a). In some embodiments, the first extracellular antigen binding domain comprises the amino acid sequence of SEQ ID NO: 147. In some embodiments, the first extracellular antigen binding domain is derived from an antibody or antigen binding fragment thereof targeting NKG2D ligand or a variant thereof. In some embodiments, the NKG2D ligand is selected from a group consisting of MICA/B, ULBP-1, ULBP-2,5,6 and ULBP-3. In some embodiments, the antibody or antigen binding fragment thereof comprises one or more scFv(s) or one or more sdAb(s) that bind to the NKG2D ligand. In some embodiments, the extracellular antigen binding domain is derived from an extracellular domain of an immune checkpoint. In some embodiments, the immune checkpoint is selected from a group consisting of PD-1, CTLA4, or a variant thereof. In some embodiments, the immune checkpoint is PD-1. In some embodiments, the immune checkpoint is CTLA4.

In some embodiments, the extracellular antigen binding domain of the chimeric cytokine receptor provided herein is monospecific. In other embodiments, the extracellular antigen binding domain of the chimeric cytokine receptor provided herein is multispecific. In other embodiments, the extracellular antigen binding domain of the chimeric cytokine receptor provided herein is monovalent. In other embodiments, the extracellular antigen binding domain of the chimeric cytokine receptor provided herein is multivalent. In some embodiments, the extracellular antigen binding domain comprises two or more antigen binding domains which are fused to each other directly via peptide bonds, or via peptide linkers.

In some embodiments, the extracellular antigen binding domain comprises an antibody or a fragment thereof. For example, the binding domain may be derived from monoclonal antibodies (including agonist, antagonist, neutralizing antibodies, full length or intact monoclonal antibodies), antibody with polyepitopic or monoepitopic specificity, polyclonal or monovalent antibodies, multivalent antibodies, multispecific antibodies (e.g., bispecific antibodies so long as they exhibit the desired biological activity), formed from at least two intact antibodies, single chain antibodies, single domain antibodies and fragments thereof (e.g., domain antibodies). An antibody can be human, humanized, chimeric and/or affinity matured, as well as an antibody from other species, for example, mouse, rabbit, llama, etc. In some embodiments, the antibody include a polypeptide product of B cells within the immunoglobulin class of polypeptides that is able to bind to a specific molecular antigen and is composed of two identical pairs of polypeptide chains, wherein each pair has one heavy chain (about 50-70 kDa) and one light chain (about 25 kDa), each amino-terminal portion of each chain includes a variable region of about 100 to about 130 or more amino acids, and each carboxy-terminal portion of each chain includes a constant region. See, e.g., Antibody Engineering (Borrebaeck ed., 2d ed. 1995); and Kuby, Immunology (3d ed. 1997). Antibodies also include, but are not limited to, synthetic antibodies, recombinantly produced antibodies, single domain antibodies including from Camelidae species (e.g., llama or alpaca) or their humanized variants, intrabodies, anti-idiotypic (anti-Id) antibodies, and functional fragments (e.g., antigen-binding fragments) of any of the above, which refers to a portion of an antibody heavy or light chain polypeptide that retains some or all of the binding activity of the antibody from which the fragment was derived. Non-limiting examples of functional fragments (e.g., antigen-binding fragments) include single-chain Fvs (scFv) (e.g., including monospecific, bispecific, etc.), Fab fragments, F(ab′) fragments, F(ab)₂ fragments, F(ab′)₂ fragments, disulfide-linked Fvs (dsFv), Fd fragments, Fv fragments, diabody, triabody, tetrabody, and minibody. In particular, antibodies provided herein include immunoglobulin molecules and immunologically active portions of immunoglobulin molecules, for example, antigen-binding domains or molecules that contain an antigen-binding site that binds to an antigen (e.g., one or more CDRs of an antibody). Such antibody fragments can be found in, for example, Harlow and Lane, Antibodies: A Laboratory Manual (1989); Mol. Biology and Biotechnology: A Comprehensive Desk Reference (Myers ed., 1995); Huston et al., 1993, Cell Biophysics 22:189-224; Pluckthun and Skerra, 1989, Meth. Enzymol. 178:497-515; and Day, Advanced Immunochemistry (2d ed. 1990). The antibodies provided herein can be of any class (e.g., IgG, IgE, IgM, IgD, and IgA) or any subclass (e.g., IgG1, IgG2, IgG3, IgG4, IgA1, and IgA2) of immunoglobulin molecule. Antibodies may be agonistic antibodies or antagonistic antibodies. Antibodies may be neither agonistic nor antagonistic.

In a specific embodiment, the extracellular antigen binding domain of the present chimeric cytokine receptors comprise a single-chain Fv (sFv or scFv). ScFvs are antibody fragments that comprise the VH and VL antibody domains connected into a single polypeptide chain. Preferably, the scFv polypeptide further comprises a polypeptide linker between the VH and VL domains which enables the scFv to form the desired structure for antigen binding. See Pluckthun in The Pharmacology of Monoclonal Antibodies, vol. 113, Rosenburg and Moore eds., Springer-Verlag, New York, pp. 269-315 (1994).

In another specific embodiment, the extracellular antigen binding domain of the present chimeric cytokine receptors comprises one or more single domain antibodies (sdAbs). The sdAbs may be of the same or different origins, and of the same or different sizes. Exemplary sdAbs include, but are not limited to, heavy chain variable domains from heavy-chain only antibodies (e.g., V_{H}H or VNAR), binding molecules naturally devoid of light chains, single domains (such as VH or VL) derived from conventional 4-chain antibodies, humanized heavy-chain only antibodies, human single domain antibodies produced by transgenic mice or rats expressing human heavy chain segments, and engineered domains and single domain scaffolds other than those derived from antibodies. Any sdAbs known in the art or developed by the present disclosure, including the single domain antibodies described above in the present disclosure, may be used to construct the chimeric cytokine receptors described herein. The sdAbs may be derived from any species including, but not limited to mouse, rat, human, camel, llama, lamprey, fish, shark, goat, rabbit, and bovine. Single domain antibodies contemplated herein also include naturally occurring single domain antibody molecules from species other than Camelidae and sharks.

In some embodiments, the sdAb is derived from a naturally occurring single domain antigen binding molecule known as heavy chain antibody devoid of light chains (also referred herein as “heavy chain only antibodies”). Such single domain molecules are disclosed in WO 94/04678 and Hamers-Casterman, C. et al., Nature 363:446-448 (1993), for example. For clarity reasons, the variable domain derived from a heavy chain molecule naturally devoid of light chain is known herein as a V_{H}H to distinguish it from the conventional VH of four chain immunoglobulins. Such a V_{H}H molecule can be derived from antibodies raised in Camelidae species, for example, camel, llama, vicuna, fromedary, alpaca and guanaco. Other species besides Camelidae may produce heavy chain molecules naturally devoid of light chain, and such V_{H}Hs are within the scope of the present disclosure. In addition, humanized versions of V_{H}Hs as well as other modifications and variants are also contemplated and within the scope of the present disclosure. In some embodiments, the sdAb is derived from a variable region of the immunoglobulin found in cartilaginous fish. For example, the sdAb can be derived from the immunoglobulin isotype known as Novel Antigen Receptor (NAR) found in the serum of shark. Methods of producing single domain molecules derived from a variable region of NAR (“IgNARs”) are described in WO 03/014161 and Streltsov, Protein Sci. 14:2901-2909 (2005).

In some embodiments, naturally occurring V_{H}H domains against a particular antigen or target, can be obtained from (naïve or immune) libraries of Camelid V_{H}H sequences. Such methods may or may not involve screening such a library using said antigen or target, or at least one part, fragment, antigenic determinant or epitope thereof using one or more screening techniques known in the field. Such libraries and techniques are for example described in WO 99/37681, WO 01/90190, WO 03/025020 and WO 03/035694. Alternatively, improved synthetic or semi-synthetic libraries derived from (naïve or immune) V_{H}H libraries may be used, such as V_{H}H libraries obtained from (naïve or immune) V_{H}H libraries by techniques such as random mutagenesis and/or CDR shuffling, as for example described in WO 00/43507.

In some embodiments, the sdAb is recombinant, CDR-grafted, humanized, camelized, de-immunized and/or in vitro generated (e.g., selected by phage display). In some embodiments, the amino acid sequence of the framework regions may be altered by “camelization” of specific amino acid residues in the framework regions. Camelization refers to the replacing or substitution of one or more amino acid residues in the amino acid sequence of a (naturally occurring) VH domain from a conventional 4-chain antibody by one or more of the amino acid residues that occur at the corresponding position(s) in a V_{H}H domain of a heavy chain antibody. This can be performed in a manner known in the field, which will be clear to the skilled person. Such “camelizing” substitutions are preferably inserted at amino acid positions that form and/or are present at the VH-VL interface, and/or at the so-called Camelidae hallmark residues, as defined herein (see for example WO 94/04678, Davies and Riechmann FEBS Letters 339: 285-290 (1994); Davies and Riechmann, Protein Engineering 9 (6): 531-537 (1996); Riechmann, J. Mol. Biol. 259: 957-969 (1996); and Riechmann and Muyldermans, J. Immunol. Meth. 231: 25-38 (1999)).

In some embodiments, the sdAb is a human single domain antibody produced by transgenic mice or rats expressing human heavy chain segments. See, e.g., US20090307787, U.S. Pat. No. 8,754,287, US20150289489, US20100122358, and WO2004049794.

In some embodiments, the single domain antibodies are generated from conventional four-chain antibodies. See, for example, EP 0 368 684; Ward et al., Nature, 341 (6242): 544-6 (1989); Holt et al., Trends Biotechnol., 21(11):484-490 (2003); WO 06/030220; and WO 06/003388.

In some embodiments, the extracellular antigen binding domain comprises humanized antibodies or fragment thereof. A humanized antibody can comprise human framework region and human constant region sequences.

Humanized antibodies can be produced using a variety of techniques known in the art, including but not limited to, CDR-grafting (European Patent No. EP 239,400; International publication No. WO 91/09967; and U.S. Pat. Nos. 5,225,539, 5,530,101, and 5,585,089), veneering or resurfacing (European Patent Nos. EP 592,106 and EP 519,596; Padlan, 1991, Molecular Immunology 28(4/5):489-498; Studnicka et al., 1994, Protein Engineering 7(6):805-814; and Roguska et al., 1994, PNAS 91:969-973), chain shuffling (U.S. Pat. No. 5,565,332), and techniques disclosed in, e.g., U.S. Pat. Nos. 6,407,213, 5,766,886, WO 93/17105, Tan et al., J. Immunol. 169:1119 25 (2002), Caldas et al., Protein Eng. 13(5):353-60 (2000), Morea et al., Methods 20(3):267 79 (2000), Baca et al., J. Biol. Chem. 272(16):10678-84 (1997), Roguska et al., Protein Eng. 9(10):895 904 (1996), Couto et al., Cancer Res. 55 (23 Supp):5973s-5977s (1995), Couto et al., Cancer Res. 55(8):1717-22 (1995), Sandhu J S, Gene 150(2):409-10 (1994), and Pedersen et al., J. Mol. Biol. 235(3):959-73 (1994). See also U.S. Patent Pub. No. US 2005/0042664 A1 (Feb. 24, 2005), each of which is incorporated by reference herein in its entirety. Various methods for humanizing non-human antibodies are known in the art. For example, a humanized antibody can have one or more amino acid residues introduced into it from a source that is non-human. These non-human amino acid residues are often referred to as “import” residues, which are typically taken from an “import” variable domain. Humanization may be performed, for example, following the method of Jones et al., 1986, Nature 321:522-25; Riechmann et al., 1988, Nature 332:323-27; and Verhoeyen et al., 1988, Science 239:1534-36), by substituting hypervariable region sequences for the corresponding sequences of a human antibody.

In some cases, the humanized antibodies are constructed by CDR grafting, in which the amino acid sequences of the six CDRs of the parent non-human antibody (e.g., rodent) are grafted onto a human antibody framework. For example, Padlan et al. determined that only about one third of the residues in the CDRs actually contact the antigen, and termed these the “specificity determining residues,” or SDRs (Padlan et al., 1995, FASEB J. 9:133-39). In the technique of SDR grafting, only the SDR residues are grafted onto the human antibody framework (see, e.g., Kashmiri et al., 2005, Methods 36:25-34).

The choice of human variable domains, both light and heavy, to be used in making the humanized antibodies can be important to reduce antigenicity. For example, according to the so-called “best-fit” method, the sequence of the variable domain of a non-human (e.g., rodent) antibody is screened against the entire library of known human variable-domain sequences. The human sequence that is closest to that of the rodent may be selected as the human framework for the humanized antibody (Sims et al., 1993, J. Immunol. 151:2296-308; and Chothia et al., 1987, J. Mol. Biol. 196:901-17). Another method uses a particular framework derived from the consensus sequence of all human antibodies of a particular subgroup of light or heavy chains. The same framework may be used for several different humanized antibodies (Carter et al., 1992, Proc. Natl. Acad. Sci. USA 89:4285-89; and Presta et al., 1993, J. Immunol. 151:2623-32). In some cases, the framework is derived from the consensus sequences of the most abundant human subclasses, VL6 subgroup I (VL6I) and VH subgroup III (VHIII). In another method, human germline genes are used as the source of the framework regions.

In an alternative paradigm based on comparison of CDRs, called superhumanization, FR homology is irrelevant. The method consists of comparison of the non-human sequence with the functional human germline gene repertoire. Those genes encoding the same or closely related canonical structures to the murine sequences are then selected. Next, within the genes sharing the canonical structures with the non-human antibody, those with highest homology within the CDRs are chosen as FR donors. Finally, the non-human CDRs are grafted onto these FRs (see, e.g., Tan et al., 2002, J. Immunol. 169:1119-25).

It is further generally desirable that antibodies be humanized with retention of their affinity for the antigen and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. These include, for example, WAM (Whitelegg and Rees, 2000, Protein Eng. 13:819-24), Modeller (Sali and Blundell, 1993, J. Mol. Biol. 234:779-815), and Swiss PDB Viewer (Guex and Peitsch, 1997, Electrophoresis 18:2714-23). Inspection of these displays permits analysis of the likely role of the residues in the functioning of the candidate immunoglobulin sequence, e.g., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Another method for antibody humanization is based on a metric of antibody humanness termed Human String Content (HSC). This method compares the mouse sequence with the repertoire of human germline genes, and the differences are scored as HSC. The target sequence is then humanized by maximizing its HSC rather than using a global identity measure to generate multiple diverse humanized variants (Lazar et al., 2007, Mol. Immunol. 44:1986-98).

In addition to the methods described above, empirical methods may be used to generate and select humanized antibodies. These methods include those that are based upon the generation of large libraries of humanized variants and selection of the best clones using enrichment technologies or high throughput screening techniques. Antibody variants may be isolated from phage, ribosome, and yeast display libraries as well as by bacterial colony screening (see, e.g., Hoogenboom, 2005, Nat. Biotechnol. 23:1105-16; Dufner et al., 2006, Trends Biotechnol. 24:523-29; Feldhaus et al., 2003, Nat. Biotechnol. 21:163-70; and Schlapschy et al., 2004, Protein Eng. Des. Sel. 17:847-60).

In the FR library approach, a collection of residue variants are introduced at specific positions in the FR followed by screening of the library to select the FR that best supports the grafted CDR. The residues to be substituted may include some or all of the “Vernier” residues identified as potentially contributing to CDR structure (see, e.g., Foote and Winter, 1992, J. Mol. Biol. 224:487-99), or from the more limited set of target residues identified by Baca et al. (1997, J. Biol. Chem. 272:10678-84).

In FR shuffling, whole FRs are combined with the non-human CDRs instead of creating combinatorial libraries of selected residue variants (see, e.g., Dall'Acqua et al., 2005, Methods 36:43-60). The libraries may be screened for binding in a two-step process, first humanizing VL, followed by VH. Alternatively, a one-step FR shuffling process may be used. Such a process has been shown to be more efficient than the two-step screening, as the resulting antibodies exhibited improved biochemical and physicochemical properties including enhanced expression, increased affinity, and thermal stability (see, e.g., Damschroder et al., 2007, Mol. Immunol. 44:3049-60).

The “humaneering” method is based on experimental identification of essential minimum specificity determinants (MSDs) and is based on sequential replacement of non-human fragments into libraries of human FRs and assessment of binding. It begins with regions of the CDR3 of non-human VH and VL chains and progressively replaces other regions of the non-human antibody into the human FRs, including the CDR1 and CDR2 of both VH and VL. This methodology typically results in epitope retention and identification of antibodies from multiple subclasses with distinct human V-segment CDRs. Humaneering allows for isolation of antibodies that are 91-96% homologous to human germline gene antibodies (see, e.g., Alfenito, Cambridge Healthtech Institute's Third Annual PEGS, The Protein Engineering Summit, 2007).

The “human engineering” method involves altering a non-human antibody or antibody fragment, such as a mouse or chimeric antibody or antibody fragment, by making specific changes to the amino acid sequence of the antibody so as to produce a modified antibody with reduced immunogenicity in a human that nonetheless retains the desirable binding properties of the original non-human antibodies. Generally, the technique involves classifying amino acid residues of a non-human (e.g., mouse) antibody as “low risk,” “moderate risk,” or “high risk” residues. The classification is performed using a global risk/reward calculation that evaluates the predicted benefits of making particular substitution (e.g., for immunogenicity in humans) against the risk that the substitution will affect the resulting antibody's folding. The particular human amino acid residue to be substituted at a given position (e.g., low or moderate risk) of a non-human (e.g., mouse) antibody sequence can be selected by aligning an amino acid sequence from the non-human antibody's variable regions with the corresponding region of a specific or consensus human antibody sequence. The amino acid residues at low or moderate risk positions in the non-human sequence can be substituted for the corresponding residues in the human antibody sequence according to the alignment. Techniques for making human engineered proteins are described in greater detail in Studnicka et al., 1994, Protein Engineering 7:805-14; U.S. Pat. Nos. 5,766,886; 5,770,196; 5,821,123; and 5,869,619; and PCT Publication WO 93/11794.

A composite human antibody can be generated using, for example, Composite Human Antibody™ technology (Antitope Ltd., Cambridge, United Kingdom). To generate composite human antibodies, variable region sequences are designed from fragments of multiple human antibody variable region sequences in a manner that avoids T cell epitopes, thereby minimizing the immunogenicity of the resulting antibody. Such antibodies can comprise human constant region sequences, e.g., human light chain and/or heavy chain constant regions.

A deimmunized antibody is an antibody in which T-cell epitopes have been removed. Methods for making deimmunized antibodies have been described. See, e.g., Jones et al., Methods Mol Biol. 2009; 525:405-23, xiv, and De Groot et al., Cell. Immunol. 244:148-153(2006)). Deimmunized antibodies comprise T-cell epitope-depleted variable regions and human constant regions. Briefly, VH and VL of an antibody are cloned and T-cell epitopes are subsequently identified by testing overlapping peptides derived from the VH and VL of the antibody in a T cell proliferation assay. T cell epitopes are identified via in silico methods to identify peptide binding to human MHC class II. Mutations are introduced in the VH and VL to abrogate binding to human MHC class II. Mutated VH and VL are then utilized to generate the deimmunized antibody.

In certain embodiments, the extracellular antigen binding domain comprises multiple binding domains. In some embodiments, the extracellular antigen binding domain comprises multispecific antibodies or fragments thereof, e.g., an extracellular antigen binding domain comprising multiple binding domains (e.g., multiple scFvs) in tandem. In other embodiments, the extracellular antigen binding domain comprises multivalent antibodies or fragments thereof. The term “specificity” refers to selective recognition of an antigen binding protein for a particular epitope of an antigen. The term “multispecific” as used herein denotes that an antigen binding protein has two or more antigen-binding sites of which at least two bind different antigens. The term “valent” as used herein denotes the presence of a specified number of binding sites in an antigen binding protein. A full length antibody has two binding sites and is bivalent. As such, the terms “trivalent”, “tetravalent”, “pentavalent” and “hexavalent” denote the presence of two binding site, three binding sites, four binding sites, five binding sites, and six binding sites, respectively, in an antigen binding protein.

Multispecific antibodies such as bispecific antibodies are antibodies that have binding specificities for at least two different antigens. Methods for making multipecific antibodies are known in the art, such as, by co-expression of two immunoglobulin heavy chain-light chain pairs, where the two heavy chains have different specificities (see, e.g., Milstein and Cuello, 1983, Nature 305:537-40). For further details of generating multispecific antibodies (e.g., bispecific antibodies), see, for example, Bispecific Antibodies (Kontermann ed., 2011).

The antibodies can be multivalent antibodies with two or more antigen binding sites (e.g., tetravalent antibodies), which can be readily produced by recombinant expression of nucleic acid encoding the polypeptide chains of the antibody. In certain embodiments, a multivalent antibody comprises (or consists of) three to about eight antigen binding sites. In one such embodiment, a multivalent antibody comprises (or consists of) four antigen binding sites. The multivalent antibody comprises at least one polypeptide chain (e.g., two polypeptide chains), wherein the polypeptide chain(s) comprise two or more variable domains. For instance, the polypeptide chain(s) may comprise VD1-(X1)n-VD2-(X2)n-Fc, wherein VD1 is a first variable domain, VD2 is a second variable domain, Fc is one polypeptide chain of an Fc region, X1 and X2 represent an amino acid or polypeptide, and n is 0 or 1. For instance, the polypeptide chain(s) may comprise: VH-CH1-flexible linker-VH-CH1-Fc region chain; or VH-CH1-VH-CH1-Fc region chain. The multivalent antibody herein may further comprise at least two (e.g., four) light chain variable domain polypeptides. The multivalent antibody herein may, for instance, comprise from about two to about eight light chain variable domain polypeptides. The light chain variable domain polypeptides contemplated here comprise a light chain variable domain and, optionally, further comprise a CL domain.

In case there are multiple binding domains in the extracellular antigen binding domain of the present chimeric cytokine receptors. The various domains may be fused to each other via peptide linkers. In some embodiments, the domains are directly fused to each other without any peptide linkers. The peptide linkers may be the same or different. Each peptide linker may have the same or different length and/or sequence depending on the structural and/or functional features of the various domains. Each peptide linker may be selected and optimized independently. The length, the degree of flexibility and/or other properties of the peptide linker(s) used in the chimeric cytokine receptors may have some influence on properties, including but not limited to the affinity, specificity or avidity for one or more particular antigens or epitopes. In some embodiment, a peptide linker comprises flexible residues (such as glycine and serine) so that the adjacent domains are free to move relative to each other. For example, a glycine-serine doublet can be a suitable peptide linker.

The peptide linker may have a naturally occurring sequence, or a non-naturally occurring sequence. For example, a sequence derived from the hinge region of heavy chain only antibodies may be used as the linker. See, for example, WO1996/34103. In some embodiments, the peptide linker is a flexible linker. Exemplary flexible linkers include but not limited to glycine polymers (G)n, glycine-serine polymers (including, for example, (GS)n (SEQ ID NO: 152), (GSGGS)n (SEQ ID NO: 153), (GGGS)n (SEQ ID NO: 154), and (GGGGS)n (SEQ ID NO: 155), where n is an integer of at least one), glycine-alanine polymers, alanine-serine polymers, and other flexible linkers known in the art. Other linkers known in the art, for example, as described in WO2016014789, WO2015158671, WO2016102965, US20150299317, WO2018067992, U.S. Pat. No. 7,741,465, Colcher et al., J. Nat. Cancer Inst. 82:1191-1197 (1990), and Bird et al., Science 242:423-426 (1988) may also be included in the chimeric cytokine receptors provided herein, the disclosure of each of which is incorporated herein by reference.

In some embodiments, the extracellular antigen binding domain provided in the present chimeric cytokine receptors recognizes an antigen that acts as a cell surface marker on target cells associated with a special disease state. In some embodiments, the antigen is a tumor antigen. Tumors express a number of proteins that can serve as a target antigen for an immune response, particularly T cell mediated immune responses. The antigens targeted by the chimeric cytokine receptor may be antigens on a single diseased cell or antigens that are expressed on different cells that each contribute to the disease. The antigens targeted by the chimeric cytokine receptor may be directly or indirectly involved in the diseases.

In some embodiments, the antigen of a target cell is an antigen on the surface of the cancer cell. In some embodiments, the antigen is a tumor-specific antigen, a tumor-associated antigen, or a neoantigen.

In some embodiments, the target cell is a cancer cell, e.g., a cell of an adrenal cancer, anal cancer, appendix cancer, bile duct cancer, bladder cancer, bone cancer, brain cancer, breast cancer, cervical cancer, colorectal cancer, esophageal cancer, gallbladder cancer, gestational trophoblastic, head and neck cancer, Hodgkin lymphoma, intestinal cancer, kidney cancer, leukemia, liver cancer, lung cancer, melanoma, mesothelioma, multiple myeloma (MN), neuroendocrine tumor, non-Hodgkin lymphoma, oral cancer, ovarian cancer, pancreatic cancer, prostate cancer, sinus cancer, skin cancer, soft tissue sarcoma spinal cancer, stomach cancer, testicular cancer, throat cancer, thyroid cancer, uterine cancer, endometrial cancer, vaginal cancer, or vulvar cancer. In some embodiments, the cancer is an adrenal cancer, anal cancer, appendix cancer, bile duct cancer, bladder cancer, bone cancer, brain cancer, breast cancer, cervical cancer, colorectal cancer, esophageal cancer, gallbladder cancer, gestational trophoblastic, head and neck cancer, Hodgkin lymphoma, intestinal cancer, kidney cancer, leukemia, liver cancer, lung cancer, melanoma, mesothelioma, multiple myeloma (MM), neuroendocrine tumor, non-Hodgkin lymphoma, oral cancer, ovarian cancer, pancreatic cancer, prostate cancer, sinus cancer, skin cancer, soft tissue sarcoma spinal cancer, stomach cancer, testicular cancer, throat cancer, thyroid cancer, uterine cancer, endometrial cancer, vaginal cancer, or vulvar cancer.

In some embodiments, the adrenal cancer is an adrenocortical carcinoma (ACC), adrenal cortex cancer, pheochromocytoma, or neuroblastoma. In some embodiments, the anal cancer is a squamous cell carcinoma, cloacogenic carcinoma, adenocarcinoma, basal cell carcinoma, or melanoma. In some embodiments, the appendix cancer is a neuroendocrine tumor (NET), mucinous adenocarcinoma, goblet cell carcinoid, intestinal-type adenocarcinoma, or signet-ring cell adenocarcinoma. In some embodiments, the bile duct cancer is an extrahepatic bile duct cancer, adenocarcinomas, hilar bile duct cancer, perihilar bile duct cancer, distal bile duct cancer, or intrahepatic bile duct cancer. In some embodiments, the bladder cancer is transitional cell carcinoma (TCC), papillary carcinoma, flat carcinoma, squamous cell carcinoma, adenocarcinoma, small-cell carcinoma, or sarcoma. In some embodiments, the bone cancer is a primary bone cancer, sarcoma, osteosarcoma, chondrosarcoma, sarcoma, fibrosarcoma, malignant fibrous histiocytoma, giant cell tumor of bone, chordoma, or metastatic bone cancer. In some embodiments, the brain cancer is an astrocytoma, brain stem glioma, glioblastoma, meningioma, ependymoma, oligodendroglioma, mixed glioma, pituitary carcinoma, pituitary adenoma, craniopharyngioma, germ cell tumor, pineal region tumor, medulloblastoma, or primary CNS lymphoma. In some embodiments, the breast cancer is a breast adenocarcinoma, invasive breast cancer, noninvasive breast cancer, breast sarcoma, metaplastic carcinoma, adenocystic carcinoma, phyllodes tumor, angiosarcoma, HER2-positive breast cancer, triple-negative breast cancer, or inflammatory breast cancer. In some embodiments, the cervical cancer is a squamous cell carcinoma, or adenocarcinoma. In some embodiments, the colorectal cancer is a colorectal adenocarcinoma, primary colorectal lymphoma, gastrointestinal stromal tumor, leiomyosarcoma, carcinoid tumor, mucinous adenocarcinoma, signet ring cell adenocarcinoma, gastrointestinal carcinoid tumor, or melanoma. In some embodiments, the esophageal cancer is an adenocarcinoma or squamous cell carcinoma. In some embodiments, the gall bladder cancer is an adenocarcinoma, papillary adenocarcinoma, adenosquamous carcinoma, squamous cell carcinoma, small cell carcinoma, or sarcoma. In some embodiments, the gestational trophoblastic disease (GTD) is a hydatidiform mole, gestational trophoblastic neoplasia (GTN), choriocarcinoma, placental-site trophoblastic tumor (PSTT), or epithelioid trophoblastic tumor (ETT). In some embodiments, the head and neck cancer is a laryngeal cancer, nasopharyngeal cancer, hypopharyngeal cancer, nasal cavity cancer, paranasal sinus cancer, salivary gland cancer, oral cancer, oropharyngeal cancer, or tonsil cancer. In some embodiments, the Hodgkin lymphoma is a classical Hodgkin lymphoma, nodular sclerosis, mixed cellularity, lymphocyte-rich, lymphocyte-depleted, or nodular lymphocyte-predominant Hodgkin lymphoma (NLPHL). In some embodiments, the intestinal cancer is a small intestine cancer, small bowel cancer, adenocarcinoma, sarcoma, gastrointestinal stromal tumors, carcinoid tumors, or lymphoma. In some embodiments, the kidney cancer is a renal cell carcinoma (RCC), clear cell RCC, papillary RCC, chromophobe RCC, collecting duct RCC, unclassified RCC, transitional cell carcinoma, urothelial cancer, renal pelvis carcinoma, or renal sarcoma. In some embodiments, the leukemia is an acute lymphocytic leukemia (ALL), acute myeloid leukemia (AML), chronic lymphocytic leukemia (CLL), chronic myeloid leukemia (CML), hairy cell leukemia (HCL), or a myelodysplastic syndrome (MDS). In a specific embodiment, the leukemia is AML. In some embodiments, the liver cancer is a hepatocellular carcinoma (HCC), fibrolamellar HCC, cholangiocarcinoma, angiosarcoma, or liver metastasis. In some embodiments, the lung cancer is a small cell lung cancer, small cell carcinoma, combined small cell carcinoma, non-small cell lung cancer, lung adenocarcinoma, squamous cell lung cancer, large-cell undifferentiated carcinoma, pulmonary nodule, metastatic lung cancer, adenosquamous carcinoma, large cell neuroendocrine carcinoma, salivary gland-type lung carcinoma, lung carcinoid, mesothelioma, sarcomatoid carcinoma of the lung, or malignant granular cell lung tumor. In some embodiments, the melanoma is a superficial spreading melanoma, nodular melanoma, acral-lentiginous melanoma, lentigo maligna melanoma, amelanotic melanoma, desmoplastic melanoma, ocular melanoma, or metastatic melanoma. In some embodiments, the mesothelioma is a pleural mesothelioma, peritoneal mesothelioma, pericardial mesothelioma, or testicular mesothelioma. In some embodiments, the multiple myeloma is an active myeloma or smoldering myeloma. In some embodiments, the neuroendocrine tumor is a gastrointestinal neuroendocrine tumor, pancreatic neuroendocrine tumor, or lung neuroendocrine tumor. In some embodiments, the non-Hodgkin's lymphoma is an anaplastic large-cell lymphoma, lymphoblastic lymphoma, peripheral T cell lymphoma, follicular lymphoma, cutaneous T cell lymphoma, lymphoplasmacytic lymphoma, marginal zone B-cell lymphoma, MALT lymphoma, small-cell lymphocytic lymphoma, Burkitt lymphoma, chronic lymphocytic leukemia (CLL), small lymphocytic lymphoma (SLL), precursor T-lymphoblastic leukemia/lymphoma, acute lymphocytic leukemia (ALL), adult T cell lymphoma/leukemia (ATLL), hairy cell leukemia, B-cell lymphomas, diffuse large B-cell lymphoma (DLBCL), primary mediastinal B-cell lymphoma, primary central nervous system (CNS) lymphoma, mantle cell lymphoma (MCL), marginal zone lymphomas, mucosa-associated lymphoid tissue (MALT) lymphoma, nodal marginal zone B-cell lymphoma, splenic marginal zone B-cell lymphoma, lymphoplasmacytic lymphoma, B-cell non-Hodgkin lymphoma, T cell non-Hodgkin lymphoma, natural killer cell lymphoma, cutaneous T cell lymphoma, Alibert-Bazin synfrome, Sezary synfrome, primary cutaneous anaplastic large-cell lymphoma, peripheral T cell lymphoma, angioimmunoblastic T cell lymphoma (AITL), anaplastic large-cell lymphoma (ALCL), systemic ALCL, enteropathy-type T cell lymphoma (EATL), or hepatosplenic gamma/delta T cell lymphoma. In some embodiments, the oral cancer is a squamous cell carcinoma, verrucous carcinoma, minor salivary gland carcinomas, lymphoma, benign oral cavity tumor, eosinophilic granuloma, fibroma, granular cell tumor, karatoacanthoma, leiomyoma, osteochonfroma, lipoma, schwannoma, neurofibroma, papilloma, condyloma acuminatum, verruciform xanthoma, pyogenic granuloma, rhabdomyoma, odontogenic tumors, leukoplakia, erythroplakia, squamous cell lip cancer, basal cell lip cancer, mouth cancer, gum cancer, or tongue cancer. In some embodiments, the ovarian cancer is a ovarian epithelial cancer, mucinous epithelial ovarian cancer, endometrioid epithelial ovarian cancer, clear cell epithelial ovarian cancer, undifferentiated epithelial ovarian cancer, ovarian low malignant potential tumors, primary peritoneal carcinoma, fallopian tube cancer, germ cell tumors, teratoma, dysgerminoma ovarian germ cell cancer, endodermal sinus tumor, sex cord-stromal tumors, sex cord-gonadal stromal tumor, ovarian stromal tumor, granulosa cell tumor, granulosa-theca tumor, Sertoli-Leydig tumor, ovarian sarcoma, ovarian carcinosarcoma, ovarian adenosarcoma, ovarian leiomyosarcoma, ovarian fibrosarcoma, Krukenberg tumor, or ovarian cyst. In some embodiments, the pancreatic cancer is a pancreatic exocrine gland cancer, pancreatic endocrine gland cancer, or pancreatic adenocarcinoma, islet cell tumor, or neuroendocrine tumor. In some embodiments, the prostate cancer is a prostate adenocarcinoma, prostate sarcoma, transitional cell carcinoma, small cell carcinoma, or neuroendocrine tumor. In some embodiments, the sinus cancer is a squamous cell carcinoma, mucosa cell carcinoma, adenoid cystic cell carcinoma, acinic cell carcinoma, sinonasal undifferentiated carcinoma, nasal cavity cancer, paranasal sinus cancer, maxillary sinus cancer, ethmoid sinus cancer, or nasopharynx cancer. In some embodiments, the skin cancer is a basal cell carcinoma, squamous cell carcinoma, melanoma, Merkel cell carcinoma, Kaposi sarcoma (KS), actinic keratosis, skin lymphoma, or keratoacanthoma. In some embodiments, the soft tissue cancer is an angiosarcoma, dermatofibrosarcoma, epithelioid sarcoma, Ewing's sarcoma, fibrosarcoma, gastrointestinal stromal tumors (GISTs), Kaposi sarcoma, leiomyosarcoma, liposarcoma, dedifferentiated liposarcoma (DL), myxoid/round cell liposarcoma (MRCL), well-differentiated liposarcoma (WDL), malignant fibrous histiocytoma, neurofibrosarcoma, rhabdomyosarcoma (RMS), or synovial sarcoma. In some embodiments, the spinal cancer is a spinal metastatic tumor. In some embodiments, the stomach cancer is a stomach adenocarcinoma, stomach lymphoma, gastrointestinal stromal tumors, carcinoid tumor, gastric carcinoid tumors, Type I ECL-cell carcinoid, Type II ECL-cell carcinoid, or Type III ECL-cell carcinoid. In some embodiments, the testicular cancer is a seminoma, non-seminoma, embryonal carcinoma, yolk sac carcinoma, choriocarcinoma, teratoma, gonadal stromal tumor, leydig cell tumor, or sertoli cell tumor. In some embodiments, the throat cancer is a squamous cell carcinoma, adenocarcinoma, sarcoma, laryngeal cancer, pharyngeal cancer, nasopharynx cancer, oropharynx cancer, hypopharynx cancer, laryngeal cancer, laryngeal squamous cell carcinoma, laryngeal adenocarcinoma, lymphoepithelioma, spindle cell carcinoma, verrucous cancer, undifferentiated carcinoma, or lymph node cancer. In some embodiments, the thyroid cancer is a papillary carcinoma, follicular carcinoma, Hürthle cell carcinoma, medullary thyroid carcinoma, or anaplastic carcinoma. In some embodiments, the uterine cancer is an endometrial cancer, endometrial adenocarcinoma, endometroid carcinoma, serous adenocarcinoma, adenosquamous carcinoma, uterine carcinosarcoma, uterine sarcoma, uterine leiomyosarcoma, endometrial stromal sarcoma, or undifferentiated sarcoma. In some embodiments, the vaginal cancer is a squamous cell carcinoma, adenocarcinoma, melanoma, or sarcoma. In some embodiments, the vulvar cancer is a squamous cell carcinoma or adenocarcinoma.

Tumor antigens are proteins that are produced by tumor cells that can elicit an immune response, particularly T-cell mediated immune responses. Exemplary tumor antigens include, but not limited to, a glioma-associated antigen, carcinoembryonic antigen (CEA), β-human chorionic gonadotropin, alphafetoprotein (AFP), lectin-reactive AFP, thyroglobulin, RAGE-1, MN-CAIX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-la, p53, prostein, PSMA, HER2/neu, survivin and telomerase, prostate-carcinoma tumor antigen-1 (PCTA-1), MAGE, ELF2M, neutrophil elastase, ephrinB2, insulin growth factor (IGF)-I, IGF-II, IGF-I receptor, and mesothelin.

In some embodiments, the cancer antigen is CEA, immature laminin receptor, TAG-72, HPV E6, HPV E7, BING-4, calcium-activated chloride channel 2, cyclin-B1, 9D7, EpCAM, EphA3, Her2/neu, telomerase, mesothelin, SAP-1, surviving, a BAGE family antigen, CAGE family antigen, GAGE family antigen, MAGE family antigen, SAGE family antigen, XAGE family antigen, NY-ESO-1/LAGE-1, PRAME, SSX-2, Melan-A, MART-1, Gp100, pmel17, tyrosinase, TRP-1, TRP-2, P. polypeptide, MC1R, prostate-specific antigen, β-catenin, BRCA1, BRCA2, CDK4, CML66, fibronectin, MART-2, p53, Ras, TGF-βRII, or MUC1.

In some embodiments, the tumor antigen comprises one or more antigenic cancer epitopes associated with a malignant tumor. Malignant tumors express a number of proteins that can serve as target antigens for an immune attack. These molecules include, but are not limited to, tissue-specific antigens such as MART-1, tyrosinase and gp100 in melanoma and prostatic acid phosphatase (PAP) and prostate-specific antigen (PSA) in prostate cancer. Other target molecules belong to the group of transformation-related molecules such as the oncogene HER2/Neu/ErbB-2. Yet another group of target antigens are onco-fetal antigens such as carcinoembryonic antigen (CEA).

In some embodiments, the tumor antigen is a tumor-specific antigen (TSA) or a tumor-associated antigen (TAA). A TSA is unique to tumor cells and does not occur on other cells in the body. A TAA associated antigen is not unique to a tumor cell, and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development, when the immune system is immature, and unable to respond or they may be antigens that are normally present at extremely low levels on normal cells, but which are expressed at much higher levels on tumor cells.

Non-limiting examples of TSA or TAA antigens include: differentiation antigens such as MART-1/MelanA (MART-I), gp 100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7.

Other large, protein-based antigens include TSP-180, MAGE-4, MAGE-5, MAGE-6, RAGE, NY-ESO, pl85erbB2, pl80erbB-3, c-met, nm-23HI, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS 1, SDCCAG16, TA-90\Mac-2 binding protein\cyclophilin C-associated protein, TAAL6, TAG72, TLP, and TPS.

Additional non-limiting exemplary targets of the a chimeric cytokine receptors provided herein include GPC2, CD276, Delta-like protein ligand 3 (DLL3), NY-ESO-1, melanoma associated antigen 4; survivin protein, synovial sarcoma X breakpoint protein 2, CD3, epidermal growth factor receptor (EGFR), erbb2 tyrosine kinase receptor, HER2, CEA, CD66, CD66e, ROR1, ntrkr1 tyrosine kinase receptor, GPC3, mesothelin, glutamate carboxypeptidase II, PMSA, PD-L1, folate receptor alpha, PSCA, Mucin 1, HLA antigen (such as HLA class I antigen A-2 alpha, HLA class I antigen A-11 alpha, and HLA class II antigen), c-Met, hepatocyte growth factor receptor, K-Ras GTPase (KRAS), IL-15 receptor, Kit tyrosine kinase, PDGF receptor beta, RET tyrosine kinase receptor; Raf 1 protein kinase, Raf B protein kinase, thymidylate synthase, topoisomerase II, Brachyury protein, Flt3 tyrosine kinase, VEGF, VEGF receptor (VEGF-1 receptor, VEGF-2 receptor, and VEGF-3 receptor), estrogen receptor, neoantigen, human papillomavirus E6, and heat shock protein.

In some specific embodiments, at least one target antigen of the present chimeric cytokine receptors is CD19. In other specific embodiments, at least one target antigen of the present chimeric cytokine receptors is CD20. In yet other specific embodiments, at least one target antigen of the present chimeric cytokine receptors is CD22. In yet other specific embodiments, at least one target antigen of the present chimeric cytokine receptors is BCMA. In yet other specific embodiments, at least one target antigen of the present chimeric cytokine receptors is VEGFR2. In yet other specific embodiments, at least one target antigen of the present chimeric cytokine receptors is FAP. In yet other specific embodiments, at least one target antigen of the present chimeric cytokine receptors is EpCam. In yet other specific embodiments, at least one target antigen of the present chimeric cytokine receptors is GPC3. In yet other specific embodiments, at least one target antigen of the present chimeric cytokine receptors is DLL3. In yet other specific embodiments, at least one target antigen of the present chimeric cytokine receptors is CD133. In yet other specific embodiments, at least one target antigen of the present chimeric cytokine receptors is IL13Rα. In yet other specific embodiments, at least one target antigen of the present chimeric cytokine receptors is EGFRIII. In yet other specific embodiments, at least one target antigen of the present chimeric cytokine receptors is EphA2. In yet other specific embodiments, at least one target antigen of the present chimeric cytokine receptors is Muc1. In yet other specific embodiments, at least one target antigen of the present chimeric cytokine receptors is CD70. In yet other specific embodiments, at least one target antigen of the present chimeric cytokine receptors is CD123. In yet other specific embodiments, at least one target antigen of the present chimeric cytokine receptors is ROR1. In yet other specific embodiments, at least one target antigen of the present chimeric cytokine receptors is PSMA. In yet other specific embodiments, at least one target antigen of the present chimeric cytokine receptors is CD5. In yet other specific embodiments, at least one target antigen of the present chimeric cytokine receptors is GD2. In yet other specific embodiments, at least one target antigen of the present chimeric cytokine receptors is GAP. In yet other specific embodiments, at least one target antigen of the present chimeric cytokine receptors is CD33. In yet other specific embodiments, at least one target antigen of the present chimeric cytokine receptors is CEA. In yet other specific embodiments, at least one target antigen of the present chimeric cytokine receptors is PSCA. In yet other specific embodiments, at least one target antigen of the present chimeric cytokine receptors is Her2. In yet other specific embodiments, at least one target antigen of the present chimeric cytokine receptors is Mesothelin.

In some embodiments, the chimeric cytokine receptor provided herein binds to a B cell antigen. In some embodiments, the B cell antigen is a CD1a, CD1b, CD1c, CD1d, CD2, CD5, CD6, CD9, CD11a, CD11b, CD11c, CD17, CD18, CD19, CD20, CD21, CD22, CD23, CD24, CD25, CD26, CD27, CD29, CD30, CD31, CD32a, CD32b, CD35, CD37, CD38, CD39, CD40, CD45, CD45RA, CD45RB, CD45RC, CD45RO, CD46, CD47, CD48, CD49b, CD49c, CD49d, CD50, CD52, CD53, CD54, CD55, CD58, CD60a, CD62L, CD63, CD68, CD69, CD70, CD72, CD73, CD74, CD75, CD75S, CD77, CD79a, CD79b, CD80, CD81, CD82, CD83, CD84, CD85E, CD85I, CD85J, CD86, CD92, CD95, CD97, CD98, CD99, CD100, CD102, CD108, CD119, CD120a, CD120b, CD121b, CD122, CD124, CD125, CD126, CD130, CD132, CD137, CD138, CD139, CD147, CD148, CD150, CD152, CD162, CD164, CD166, CD167a, CD170, CD171, CD175, CD175s, CD180, CD184, CD185, CD192, CD196, CD197, CD200, CD205, CD201a, CDw210b, CD212, CD213a1, CD213a2, CD 215, CD217, CD218a, CD218b, CD220, CD221, CD222, CD224, CD225, CD226, CD227, CD229, CD230, CD232, CD252, CD252, CD254, CD255, CD256, CD257 CD258, CD259, CD260, CD261, CD262, CD263, CD264, CD267, CD268, CD269, CD270, CD272, CD274, CD275, CD277, CD279, CD283, CD289, CD290, CD295, CD298, CD300, CD300c, CD305, CD306, CD307a, CD307b, CD307c, CD307d, CD307e, CD314, CD215, CD316, CD317, CD319, CD321, CD327, CD328, CD329, CD338, CD351, CD352, CD353, CD354, CD355, CD356, CD357, CD358, CD360, CD361, CD362, or CD363 antigen.

In one embodiment, target of the present chimeric cytokine receptor is a pathogen. In certain embodiments, the target cell is a cell comprising a pathogen.

In some embodiments, the pathogen causes an infectious disease selected from the group consisting of an Acute Flaccid Myelitis (AFM), Anaplasmosis, Anthrax, Babesiosis, Botulism, Brucellosis, Campylobacteriosis, Carbapenem-resistant Infection, Chancroid, Chikungunya Virus Infection, Chlamydia, Ciguatera, Difficile Infection, Perfringens, Coccidioidomycosis fungal infection, coronavirus infection, Covid-19 (SARS-CoV-2), Creutzfeldt-Jacob Disease/transmissible spongiform encephalopathy, Cryptosporidiosis (Crypto), Cyclosporiasis, Dengue 1,2,3 or 4, Diphtheria, E. coli infection/Shiga toxin-producing (STEC), Eastern Equine Encephalitis, Hemorrhagic Fever (Ebola), Ehrlichiosis, Encephalitis, Arboviral or parainfectious, Non-Polio Enterovirus, D68 Enteroviru (EV-D68), Giardiasis, Glanders, Gonococcal Infection, Granuloma inguinale, Haemophilus Influenza disease Type B (Hib or H-flu), Hantavirus Pulmonary Synfrome (HPS), Hemolytic Uremic Synfrome (HUS), Hepatitis A (Hep A), Hepatitis B (Hep B), Hepatitis C (Hep C), Hepatitis D (Hep D), Hepatitis E (Hep E), Herpes, Herpes Zoster (Shingles), Histoplasmosis infection, Human Immunodeficiency Virus/AIDS (HIV/AIDS), Human Papillomavirus (HPV), Influenza (Flu), Legionellosis (Legionnaires Disease), Leprosy (Hansens Disease), Leptospirosis, Listeriosis (Listeria), Lyme Disease, Lymphogranuloma venereum infection (LGV), Malaria, Measles, Melioidosis, Meningitis (Viral), Meningococcal Disease (Meningitis (Bacterial)), Middle East Respiratory Synfrome Coronavirus (MERS-CoV), Mumps, Norovirus, Pediculosis, Pelvic Inflammatory Disease (PID), Pertussis (Whooping Cough), Plague (Bubonic, Septicemic, Pneumonic), Pneumococcal Disease (Pneumonia), Poliomyelitis (Polio), Powassan, Psittacosis, Pthiriasis, Pustular Rash diseases (Small pox, monkeypox, cowpox), Q-Fever, Rabies, Rickettsiosis (Rocky Mountain Spotted Fever), Rubella (German Measles), Salmonellosis gastroenteritis (Salmonella), Scabies, Scombroid, Sepsis, Severe Acute Respiratory Synfrome (SARS), Shigellosis gastroenteritis (Shigella), Smallpox, Staphyloccal Infection Methicillin-resistant (MRSA), Staphylococcal Food Poisoning Enterotoxin B Poisoning (Staph Food Poisoning), Saphylococcal Infection Vancomycin Intermediate (VISA), Staphylococcal Infection Vancomycin Resistant (VRSA), Streptococcal Disease Group A (invasive) (Strep A (invasive), Streptococcal Disease, Group B (Strep-B), Streptococcal Toxic-Shock Synfrome STSS Toxic Shock, Syphilis (primary, secondary, early latent, late latent, congenital), Tetanus Infection, Trichomoniasis, Trichonosis Infection, Tuberculosis (TB), Tuberculosis Latent (LTBI), Tularemia, Typhoid Fever Group D, Vaginosis, Varicella (Chickenpox), Vibrio cholerae (Cholera), Vibriosis (Vibrio), Ebola Virus Hemorrhagic Fever, Lasa Virus Hemorrhagic Fever, Marburg Virus Hemorrhagic Fever, West Nile Virus, Yellow Fever, Yersenia, and Zika Virus Infection. In some embodiments, the infectious disease is Acute Flaccid Myelitis (AFM).

In some embodiments, the pathogen is a bacteria. In some embodiments, the bacteria is a bacteria of a Bacillus, Bartonella, Bordetella, Borrelia, Brucella, Campylobacter, Chlamydia, Chlamydophila, Clostridium, Corynebacterium, Enterococcus, Escherichia, Francisella, Haemophilus, Helicobacter, Legionella, Leptospira, Listeria, Mycobacterium, Mycoplasma, Neisseria, Pseudomonas, Rickettsia, Salmonella, Shigella, Staphylococcus, Streptococcus, Treponema, Ureaplasma, Vibrio or Yersinia genus.

In some embodiments, the pathogen is a parasite. In some embodiments, the parasite is a protozoa, helminth, or ectoparasite. In some embodiments, the protozoa is an Entamoeba, Giardia, Leishmania, Balantidium, Plasmodium, or Cryptosporidium. In some embodiments, the helminth is a trematode, cestode, acanthocephalan, or round worm. In some embodiments, the ectoparasite is a arthropod.

In some embodiments, the pathogen is a virus. In some embodiments, the virus is a virus of the adenoviridae, arenaviridae, astroviridae, bunyaviridae, caliciviridae, coronaviridae, filoviridae, flaviviridae, hepadnaviridae, hepeviridae, orthomyxoviridae, papillomaviridae, paramyxoviridae, parvoviridae, picornaviridae, polyomaviridae, poxviridae, reoviridae, retroviridae, rhabdoviridae, or togaviridae family. In some embodiments, the virus is an adenovirus, coronavirus, coxsackievirus, Epstein-Barr virus, hepatitis A virus, hepatitis B virus, hepatitis C virus, herpes simplex virus type 2, cytomegalovirus, human herpes virus type 8, human immunodeficiency virus, influenza virus, measles virus, mumps virus, human papillomavirus, parainfluenza virus, poliovirus, rabies virus, respiratory syncytial virus, rubella virus, or varicella-zoster virus.

In some specific embodiments, the extracellular antigen binding domain of the present chimeric cytokine receptor is derived from NKG2D or truncated NKG2D (such as the ECD of NKG2D or truncated NKG2D). In some embodiments, the extracellular antigen binding domain comprises the amino acid sequence of SEQ ID NOs: 6, 156 or 157, or an amino acid sequence having at least 75%, 80%, 90%, 91%, 92%, 93%, 94%, 95%, 96%, 97%, 98%, or 99% identity to SEQ ID NOs: 6, 156 or 157. In other specific embodiments, the extracellular antigen binding domain of the present chimeric cytokine receptor comprises an antibody or antigen binding fragment thereof that binds to an NKG2D ligand. In some embodiments, the extracelluar domain comprises one or more scFv(s) that bind to an NKG2D ligand. In other embodiments, the extracellular domain comprises one or more sdAb(s) (such as one or more V_{H}H domains) that bind to an NKG2D ligand. The NKG2D ligand can be selected from (but not limited to) a group consisting of MICA/B, ULBP-1, ULBP-2,5,6 and ULBP-3. In some embodiment, the NKG2D ligand is MICA/B. In some embodiment, the NKG2D ligand is ULBP-1. In some embodiment, the NKG2D ligand is ULBP-2. In some embodiment, the NKG2D ligand is ULBP-5. In some embodiment, the NKG2D ligand is ULBP-6. In some embodiment, the NKG2D ligand is ULBP-3.

The determination of percent identity between two sequences (e.g., amino acid sequences or nucleic acid sequences) can be accomplished using a mathematical algorithm. A preferred, non-limiting example of a mathematical algorithm utilized for the comparison of two sequences is the algorithm of Karlin and Altschul, Proc. Natl. Acad. Sci. U.S.A. 87:2264 2268 (1990), modified as in Karlin and Altschul, Proc. Natl. Acad. Sci. U.S.A. 90:5873 5877 (1993). Such an algorithm is incorporated into the NBLAST and XBLAST programs of Altschul et al., J. Mol. Biol. 215:403 (1990). BLAST nucleotide searches can be performed with the NBLAST nucleotide program parameters set, e.g., for score=100, word length=12 to obtain nucleotide sequences homologous to a nucleic acid molecules described herein. BLAST protein searches can be performed with the XBLAST program parameters set, e.g., to score 50, word length=3 to obtain amino acid sequences homologous to a protein molecule described herein. To obtain gapped alignments for comparison purposes, Gapped BLAST can be utilized as described in Altschul et al., Nucleic Acids Res. 25:3389 3402 (1997). Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another non-limiting example of a mathematical algorithm utilized for the comparison of sequences is the algorithm of Myers and Miller, CABIOS 4:11-17 (1998). Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

In some embodiments, amino acid sequence modification(s) or variation(s) of the polypeptides described herein are contemplated. Variations may be a substitution, deletion, or insertion of one or more codons encoding the polypeptide that results in a change in the amino acid sequence as compared with the original polypeptide.

Amino acid substitutions can be the result of replacing one amino acid with another amino acid having similar structural and/or chemical properties, such as the replacement of a leucine with a serine, e.g., conservative amino acid replacements. Standard techniques known to those of skill in the art can be used to introduce mutations in the nucleotide sequence encoding a molecule provided herein, including, for example, site-directed mutagenesis and PCR-mediated mutagenesis which results in amino acid substitutions. Insertions or deletions may optionally be in the range of about 1 to 5 amino acids. In certain embodiments, the substitution, deletion, or insertion includes fewer than 25 amino acid substitutions, fewer than 20 amino acid substitutions, fewer than 15 amino acid substitutions, fewer than 10 amino acid substitutions, fewer than 5 amino acid substitutions, fewer than 4 amino acid substitutions, fewer than 3 amino acid substitutions, or fewer than 2 amino acid substitutions relative to the original molecule. In a specific embodiment, the substitution is a conservative amino acid substitution made at one or more predicted non-essential amino acid residues. The variation allowed may be determined by systematically making insertions, deletions, or substitutions of amino acids in the sequence and testing the resulting variants for activity exhibited by the parental polypeptides.

The polypeptides generated by conservative amino acid substitutions are included in the present disclosure. In a conservative amino acid substitution, an amino acid residue is replaced with an amino acid residue having a side chain with a similar charge. As described above, families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed and the activity of the protein can be determined. Conservative (e.g., within an amino acid group with similar properties and/or side chains) substitutions may be made, so as to maintain or not significantly change the properties.

Amino acids may be grouped according to similarities in the properties of their side chains (see, e.g., Lehninger, Biochemistry 73-75 (2d ed. 1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); and (4) basic: Lys (K), Arg (R), His(H). Alternatively, naturally occurring residues may be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; and (6) aromatic: Trp, Tyr, Phe. For example, any cysteine residue not involved in maintaining the proper conformation of the peptide also may be substituted, for example, with another amino acid, such as alanine or serine, to improve the oxidative stability of the molecule and to prevent aberrant crosslinking. Non-conservative substitutions will entail exchanging a member of one of these classes for another class.

Amino acid sequence insertions include amino- and/or carboxyl-terminal fusions ranging in length from one residue to polypeptides containing a hundred or more residues, as well as intrasequence insertions of single or multiple amino acid residues. The variations can be made using methods known in the art such as oligonucleotide-mediated (site-directed) mutagenesis, alanine scanning, and PCR mutagenesis. Site-directed mutagenesis (see, e.g., Carter, Biochem J. 237:1-7 (1986); and Zoller et al., Nucl. Acids Res. 10:6487-500 (1982)), cassette mutagenesis (see, e.g., Wells et al., Gene 34:315-23 (1985)), or other known techniques can be performed on the cloned DNA to produce the polypeptide variant DNA.

### Signal Peptide

In certain embodiments, the chimeric cytokine receptor provided herein may comprise a signal peptide (also known as a signal sequence) at the N-terminus of the polypeptide. In general, signal peptides are peptide sequences that target a polypeptide to the desired site in a cell. In some embodiments, the signal peptide targets the effector molecule to the secretory pathway of the cell and will allow for integration and anchoring of the effector molecule into the lipid bilayer. Signal peptides including signal sequences of naturally occurring proteins or synthetic, non-naturally occurring signal sequences, which are compatible for use in the CARs described herein will be evident to one of skill in the art. In some embodiments, the signal peptide is derived from a molecule selected from the group consisting of CD8α, GM-CSF receptor a, and IgG1 heavy chain.

In another aspect, provided herein is a polynucleotide encoding the chimeric cytokine receptor provided herein. In another aspect, provided herein is an immune cell expressing the chimeric cytokine receptor provided herein.

### Tag Peptide

In certain embodiments, the chimeric cytokine receptor provided herein may comprises a tag. In some embodiments, the chimeric cytokine receptor may comprises two or more tags. In some embodiments, the tag linking to the chimeric cytokine receptor is different form the tag linking to the functional exogenous receptor.

### 5.2.2. Functional Exogenous Receptors

In some embodiments, the immune effector cell provided herein further comprises one or more functional exogenous receptor(s). In some embodiments, the functional exogenous receptor is a T cell receptor (TCR), a chimeric antigen receptor (CAR), a chimeric TCR (cTCR), or a T cell antigen coupler (TAC)-like chimeric receptor. In some embodiments, the functional exogenous receptor is a CAR. In other embodiments, the functional exogenous receptor is a TCR.

Any functional exogenous receptors are included in the present disclosure. Chimeric antigen receptor (CAR) is described in more detail below only as an exemplary functional exogenous receptor provided herein, but does not limit the scope of the present disclosure.

In some embodiments, the CAR in the present immune effector cells comprises a polypeptide comprising: (a) an extracellular antigen binding domain; (b) a transmembrane domain; and (c) an intracellular signaling domain.

### Extracellular Antigen Binding Domain

The extracellular antigen binding domain of the CARs described herein comprises one or more antigen binding domains. In some embodiments, the extracellular antigen binding domain of the CAR provided herein is monospecific. In other embodiments, the extracellular antigen binding domain of the CAR provided herein is multispecific. In other embodiments, the extracellular antigen binding domain of the CAR provided herein is monovalent. In other embodiments, the extracellular antigen binding domain of the CAR provided herein is multivalent. In some embodiments, the extracellular antigen binding domain comprises two or more antigen binding domains which are fused to each other directly via peptide bonds, or via peptide linkers. In some embodiments, the extracellular antigen binding domain comprises an antibody or a fragment thereof as described in the context of the extracelluar domain of the chimeric cytokine receptor in Section 5.2.1 above. In case there are multiple binding domains in the extracellular antigen binding domain of the present CARs. The various domains may be fused to each other via peptide linkers. In some embodiments, the domains are directly fused to each other without any peptide linkers. The peptide linkers may be the same or different. Each peptide linker may have the same or different length and/or sequence depending on the structural and/or functional features of the various domains. Each peptide linker may be selected and optimized independently. The length, the degree of flexibility and/or other properties of the peptide linker(s) used in the CARs may have some influence on properties, including but not limited to the affinity, specificity or avidity for one or more particular antigens or epitopes. In some embodiment, a peptide linker comprises flexible residues (such as glycine and serine) so that the adjacent domains are free to move relative to each other. For example, a glycine-serine doublet can be a suitable peptide linker. Additional description of the applicable linkers used herein is provided in Section 5.2.1 above.

In some embodiments, the extracellular antigen binding domain provided in the present CARs recognizes an antigen that acts as a cell surface marker on target cells associated with a special disease state. In some embodiments, the antigen is a tumor antigen. Tumors express a number of proteins that can serve as a target antigen for an immune response, particularly T cell mediated immune responses. The antigens targeted by the CAR may be antigens on a single diseased cell or antigens that are expressed on different cells that each contribute to the disease. The antigens targeted by the CAR may be directly or indirectly involved in the diseases.

In some embodiments, the possible antiges targeted by the CAR provided herein is provided above in Section 5.2.1. In some embodiments, the target cell is a cancer cell. In one embodiment, target of the present CAR is a pathogen. In certain embodiments, the target cell is a cell comprising a pathogen. In some embodiments, the pathogen causes an infectious disease. In some embodiments, the pathogen is a bacteria. In some embodiments, the pathogen is a parasite. In some embodiments, the pathogen is a virus.

### Transmembrane Domain

The CARs of the present disclosure comprise a transmembrane domain that can be directly or indirectly fused to the extracellular antigen binding domain. The transmembrane domain may be derived either from a natural or from a synthetic source. Transmembrane domains compatible for use in the CARs described herein may be obtained from a naturally occurring protein. Alternatively, it can be a synthetic, non-naturally occurring protein segment, e.g., a hydrophobic protein segment that is thermodynamically stable in a cell membrane.

In some embodiments, the transmembrane domain of the CAR described herein is derived from a Type I single-pass membrane protein. In some embodiments, transmembrane domains from multi-pass membrane proteins may also be compatible for use in the CARs described herein. Multi-pass membrane proteins may comprise a complex (at least 2, 3, 4, 5, 6, 7 or more) alpha helices or a beta sheet structure. In some embodiments, the N-terminus and the C-terminus of a multi-pass membrane protein are present on opposing sides of the lipid bilayer, e.g., the N-terminus of the protein is present on the cytoplasmic side of the lipid bilayer and the C-terminus of the protein is present on the extracellular side.

Transmembrane domains for use in the CARs described herein can also comprise at least a portion of a synthetic, non-naturally occurring protein segment. In some embodiments, the transmembrane domain is a synthetic, non-naturally occurring alpha helix or beta sheet. In some embodiments, the protein segment is at least approximately 20 amino acids, e.g., at least 18, 19, 20, 21, 22, 23, 24, 25, 26, 27, 28, 29, 30, or more amino acids. Examples of synthetic transmembrane domains are known in the art, for example in U.S. Pat. No. 7,052,906 and PCT Publication No. WO 2000/032776, the relevant disclosures of which are incorporated by reference herein.

The transmembrane domain provided herein may comprise a transmembrane region and a cytoplasmic region located at the C-terminal side of the transmembrane domain. The cytoplasmic region of the transmembrane domain may comprise three or more amino acids and, in some embodiments, helps to orient the transmembrane domain in the lipid bilayer. In some embodiments, one or more cysteine residues are present in the transmembrane region of the transmembrane domain. In some embodiments, one or more cysteine residues are present in the cytoplasmic region of the transmembrane domain. In some embodiments, the cytoplasmic region of the transmembrane domain comprises positively charged amino acids. In some embodiments, the cytoplasmic region of the transmembrane domain comprises the amino acids arginine, serine, and lysine.

In some embodiments, the transmembrane region of the transmembrane domain comprises hydrophobic amino acid residues. In some embodiments, the transmembrane domain of the CAR provided herein comprises an artificial hydrophobic sequence. For example, a triplet of phenylalanine, tryptophan and valine may be present at the C terminus of the transmembrane domain. In some embodiments, the transmembrane region comprises mostly hydrophobic amino acid residues, such as alanine, leucine, isoleucine, methionine, phenylalanine, tryptophan, or valine. In some embodiments, the transmembrane region is hydrophobic. In some embodiments, the transmembrane region comprises a poly-leucine-alanine sequence. The hydropathy, or hydrophobic or hydrophilic characteristics of a protein or protein segment, can be assessed by any method known in the art, for example the Kyte and Doolittle hydropathy analysis.

In some embodiments, the transmembrane domain of the CAR comprises a transmembrane domain chosen from the transmembrane domain of an alpha, beta or zeta chain of a T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL-2R beta, IL-2R gamma, IL-7R a, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CDIOO (SEMA4D), SLAMF6 (NTB-A, Lyl08), SLAM (SLAMF1, CD150, IPO-3), BLAME (SLAMF8), SELPLG (CD162), LTBR, PAG/Cbp, NKp44, NKp30, NKp46, NKG2D, and/or NKG2C.

In some specific embodiments, the transmembrane domain is derived from CD8. In some specific embodiments, the transmembrane domain is derived from CD8α. In other specific embodiments, the transmembrane domain is derived from CD28.

### Intracellular Signaling Domain

The intracellular signaling domain in the CARs provided herein is responsible for activation of at least one of the normal effector functions of the immune effector cell expressing the CARs. The term “effector function” refers to a specialized function of a cell. Effector function of a T cell, for example, may be cytolytic activity or helper activity including the secretion of cytokines. Thus the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

In some embodiments, the intracellular signaling domain comprises a primary intracellular signaling domain of an immune effector cell. In some embodiments, the CAR comprises an intracellular signaling domain consisting essentially of a primary intracellular signaling domain of an immune effector cell. “Primary intracellular signaling domain” refers to cytoplasmic signaling sequence that acts in a stimulatory manner to induce immune effector functions. In some embodiments, the primary intracellular signaling domain contains a signaling motif known as immunoreceptor tyrosine-based activation motif, or ITAM. An “ITAM,” as used herein, is a conserved protein motif that is generally present in the tail portion of signaling molecules expressed in many immune cells. The motif may comprises two repeats of the amino acid sequence YxxL/I separated by 6-8 amino acids, wherein each x is independently any amino acid, producing the conserved motif YxxL/Ix(6-8)YxxL/I. ITAMs within signaling molecules are important for signal transduction within the cell, which is mediated at least in part by phosphorylation of tyrosine residues in the ITAM following activation of the signaling molecule. ITAMs may also function as docking sites for other proteins involved in signaling pathways. Exemplary ITAM-containing primary cytoplasmic signaling sequences include those derived from CD3z, FcR gamma (FCER1G), FcR beta (Fc Epsilon Rib), CD3 gamma, CD3 delta, CD3 epsilon, CD5, CD22, CD79a, CD79b, and CD66d.

### Co-Stimulatory Signaling Domain

In some embodiments, the CAR comprises at least one co-stimulatory signaling domain. The term “co-stimulatory signaling domain,” as used herein, refers to at least a portion of a protein that mediates signal transduction within a cell to induce an immune response such as an effector function. Many immune effector cells require co-stimulation, in addition to stimulation of an antigen-specific signal, to promote cell proliferation, differentiation and survival, as well as to activate effector functions of the cell.

The co-stimulatory signaling domain of the chimeric receptor described herein can be an intracellular signaling domain from a co-stimulatory protein, which transduces a signal and modulates responses mediated by immune cells, such as T cells, NK cells, macrophages, neutrophils, or eosinophils. “Co-stimulatory signaling domain” can be the cytoplasmic portion of a co-stimulatory molecule. The term “co-stimulatory molecule” refers to a cognate binding partner on an immune cell (such as T cell) that specifically binds with a co-stimulatory ligand, thereby mediating a co-stimulatory response by the immune cell, such as, but not limited to, proliferation and survival.

In some embodiments, the intracellular signaling domain comprises a single co-stimulatory signaling domain. In some embodiments, the intracellular signaling domain comprises two or more (such as about any of 2, 3, 4, or more) co-stimulatory signaling domains. In some embodiments, the intracellular signaling domain comprises two or more of the same co-stimulatory signaling domains. In some embodiments, the intracellular signaling domain comprises two or more co-stimulatory signaling domains from different co-stimulatory proteins, such as any two or more co-stimulatory proteins described herein. In some embodiments, the intracellular signaling domain comprises a primary intracellular signaling domain (such as intracellular signaling domain of CD3z) and one or more co-stimulatory signaling domains. In some embodiments, the one or more co-stimulatory signaling domains and the primary intracellular signaling domain (such as intracellular signaling domain of CD3z) are fused to each other via optional peptide linkers. The primary intracellular signaling domain, and the one or more co-stimulatory signaling domains may be arranged in any suitable order. In some embodiments, the one or more co-stimulatory signaling domains are located between the transmembrane domain and the primary intracellular signaling domain (such as intracellular signaling domain of CD3z). Multiple co-stimulatory signaling domains may provide additive or synergistic stimulatory effects.

Activation of a co-stimulatory signaling domain in a host cell (e.g., an immune cell) may induce the cell to increase or decrease the production and secretion of cytokines, phagocytic properties, proliferation, differentiation, survival, and/or cytotoxicity. The co-stimulatory signaling domain of any co-stimulatory molecule may be compatible for use in the CARs described herein. The type(s) of co-stimulatory signaling domain is selected based on factors such as the type of the immune effector cells in which the effector molecules would be expressed (e.g., T cells, NK cells, macrophages, neutrophils, or eosinophils) and the desired immune effector function (e.g., ADCC effect). Examples of co-stimulatory signaling domains for use in the CARs can be the intracellular signaling domain of co-stimulatory proteins, including, without limitation, members of the B7/CD28 family (e.g., B7-1/CD80, B7-2/CD86, B7-H1/PD-L1, B7-H2, B7-H3, B7-H4, B7-H6, B7-H7, BTLA/CD272, CD28, CTLA-4, Gi24/VISTA/B7-H5, ICOS/CD278, PD-1, PD-L2/B7-DC, and PDCD6); members of the TNF superfamily (e.g., 4-1BB/TNFSF9/CD137, 4-1BB Ligand/TNFSF9, BAFF/BLyS/TNFSF13B, BAFF R/TNFRSF13C, CD27/TNFRSF7, CD27 Ligand/TNFSF7, CD30/TNFRSF8, CD30 Ligand/TNFSF8, CD40/TNFRSF5, CD40/TNFSF5, CD40 Ligand/TNFSF5, DR3/TNFRSF25, GITR/TNFRSF18, GITR Ligand/TNFSF18, HVEM/TNFRSF14, LIGHT/TNFSF14, Lymphotoxin-alpha/TNF-beta, OX40/TNFRSF4, OX40 Ligand/TNFSF4, RELT/TNFRSF19L, TACI/TNFRSF13B, TL1A/TNFSF15, TNF-alpha, and TNF RII/TNFRSF1B); members of the SLAM family (e.g., 2B4/CD244/SLAMF4, BLAME/SLAMF8, CD2, CD2F-10/SLAMF9, CD48/SLAMF2, CD58/LFA-3, CD84/SLAMF5, CD229/SLAMF3, CRACC/SLAMF7, NTB-A/SLAMF6, and SLAM/CD150); and any other co-stimulatory molecules, such as CD2, CD7, CD53, CD82/Kai-1, CD90/Thy1, CD96, CD160, CD200, CD300a/LMIR1, HLA Class I, HLA-DR, Ikaros, Integrin alpha 4/CD49d, Integrin alpha 4 beta 1, Integrin alpha 4 beta 7/LPAM-1, LAG-3, TCL1A, TCL1B, CRTAM, DAP12, Dectin-1/CLEC7A, DPPIV/CD26, EphB6, TIM-1/KIM-1/HAVCR, TIM-4, TSLP, TSLP R, lymphocyte function associated antigen-1 (LFA-1), and NKG2C. In some embodiments, the one or more co-stimulatory signaling domains are selected from the group consisting of CD27, CD28, CD137, OX40, CD30, CD40, CD3, lymphocyte function-associated antigen-1(LFA-1), CD2, CD7, LIGHT, NKG2C, B7-H3 and ligands that specially bind to CD83.

In some embodiments, the co-stimulatory signaling domains are variants of any of the co-stimulatory signaling domains described herein, such that the co-stimulatory signaling domain is capable of modulating the immune response of the immune cell. In some embodiments, the co-stimulatory signaling domains comprises up to 10 amino acid residue variations (e.g., 1, 2, 3, 4, 5, or 8) as compared to a wild-type counterpart. Such co-stimulatory signaling domains comprising one or more amino acid variations may be referred to as variants. Mutation of amino acid residues of the co-stimulatory signaling domain may result in an increase in signaling transduction and enhanced stimulation of immune responses relative to co-stimulatory signaling domains that do not comprise the mutation. Mutation of amino acid residues of the co-stimulatory signaling domain may result in a decrease in signaling transduction and reduced stimulation of immune responses relative to co-stimulatory signaling domains that do not comprise the mutation.

### Signal Peptide

In certain embodiments, the CARs provided herein may comprise a signal peptide (also known as a signal sequence) at the N-terminus of the polypeptide. In general, signal peptides are peptide sequences that target a polypeptide to the desired site in a cell. In some embodiments, the signal peptide targets the effector molecule to the secretory pathway of the cell and will allow for integration and anchoring of the effector molecule into the lipid bilayer. Signal peptides including signal sequences of naturally occurring proteins or synthetic, non-naturally occurring signal sequences, which are compatible for use in the CARs described herein will be evident to one of skill in the art. In some embodiments, the signal peptide is derived from a molecule selected from the group consisting of CD8α, GM-CSF receptor a, and IgG1 heavy chain.

### Hinge Region

In some embodiments, the CARs provided herein comprise a hinge domain that is located between the extracellular antigen binding domain and the transmembrane domain. A hinge domain is an amino acid segment that is generally found between two domains of a protein and may allow for flexibility of the protein and movement of one or both of the domains relative to one another. Any amino acid sequence that provides such flexibility and movement of the extracellular antigen binding domain relative to the transmembrane domain of the effector molecule can be used.

Hinge domains of antibodies, such as an IgG, IgA, IgM, IgE, or IgD antibodies, are also compatible for use in the pH-dependent chimeric receptor systems described herein. In some embodiments, the hinge domain is the hinge domain that joins the constant domains CH1 and CH2 of an antibody. In some embodiments, the hinge domain is of an antibody and comprises the hinge domain of the antibody and one or more constant regions of the antibody. In some embodiments, the hinge domain comprises the hinge domain of an antibody and the CH3 constant region of the antibody. In some embodiments, the hinge domain comprises the hinge domain of an antibody and the CH2 and CH3 constant regions of the antibody. In some embodiments, the antibody is an IgG, IgA, IgM, IgE, or IgD antibody. In some embodiments, the antibody is an IgG antibody. In some embodiments, the antibody is an IgG1, IgG2, IgG3, or IgG4 antibody. In some embodiments, the hinge region comprises the hinge region and the CH2 and CH3 constant regions of an IgG1 antibody. In some embodiments, the hinge region comprises the hinge region and the CH3 constant region of an IgG1 antibody.

Non-naturally occurring peptides may also be used as hinge domains for the chimeric receptors described herein. In some embodiments, the hinge domain between the C-terminus of the extracellular ligand-binding domain of an Fc receptor and the N-terminus of the transmembrane domain is a peptide linker, such as a (G×S)n linker, wherein x and n, independently can be an integer between 3 and 12, including 3, 4, 5, 6, 7, 8, 9, 10, 11, 12, or more.

The hinge domain may contain about 10-100 amino acids, e.g., about any one of 15-75 amino acids, 20-50 amino acids, or 30-60 amino acids. In some embodiments, the hinge domain may be at least about any one of 10, 11, 12, 13, 14, 15, 16, 17, 18, 19, 20, 21, 22, 23, 24, 25, 26, 27, 28, 29, 30, 35, 40, 45, 50, 55, 60, 65, 70, or 75 amino acids in length.

In some embodiments, the hinge domain is a hinge domain of a naturally occurring protein. Hinge domains of any protein known in the art to comprise a hinge domain are compatible for use in the chimeric receptors described herein. In some embodiments, the hinge domain is at least a portion of a hinge domain of a naturally occurring protein and confers flexibility to the chimeric receptor.

In some specific embodiments, the hinge domain is derived from CD8α. In some embodiments, the hinge domain is a portion of the hinge domain of CD8α, e.g., a fragment containing at least 15 (e.g., 20, 25, 30, 35, or 40) consecutive amino acids of the hinge domain of CD8α. In other embodiments, the hinge domain is derived from CD28a. In some embodiments, the hinge domain is a portion of the hinge domain of CD28a, e.g., a fragment containing at least 15 (e.g., 20, 25, 30, 35, or 40) consecutive amino acids of the hinge domain of CD28a.

### Tag Peptide

In some embodiments, the functional exogenous receptor may comprises a tag. In some embodiments, the functional exogenous receptor may comprises two or more tags. In some embodiments, the tag linking to the chimeric cytokine receptor is different form the tag linking to the functional exogenous receptor.

### Exemplary CARs

Any CARs can be used in the present disclosure, including but not limited to CARs in the present disclosure.

In certain embodiments, the CAR provided herein comprises amino acid sequences with certain percent identity relative to any one of the exemplary CARs described above and in Section 6 below. In some embodiments, provided herein is a CAR comprising or consisting of an extracellular domain having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of the CARs described above and in Section 6 below.

In some embodiments, amino acid sequence modification(s) of the CARs described herein are contemplated. For example, it may be desirable to optimize the binding affinity and/or other biological properties of the extracellular domain, including but not limited to specificity, thermostability, expression level, effector functions, glycosylation, reduced immunogenicity, or solubility. Thus, in addition to the extracellular domain described herein, it is contemplated that variants of the domains described herein can be prepared. For example, scFv variants can be prepared by introducing appropriate nucleotide changes into the encoding DNA, and/or by synthesis of the desired antibody or polypeptide. Those skilled in the art who appreciate that amino acid changes may alter post-translational processes of the antibody.

Variations may be a substitution, deletion, or insertion of one or more codons encoding the polypeptide that results in a change in the amino acid sequence as compared with the original antibody or polypeptide. Sites of interest for substitutional mutagenesis include the CDRs and FRs.

Amino acid substitutions can be the result of replacing one amino acid with another amino acid having similar structural and/or chemical properties, such as the replacement of a leucine with a serine, e.g., conservative amino acid replacements. Standard techniques known to those of skill in the art can be used to introduce mutations in the nucleotide sequence encoding a molecule provided herein, including, for example, site-directed mutagenesis and PCR-mediated mutagenesis which results in amino acid substitutions. Insertions or deletions may optionally be in the range of about 1 to 5 amino acids. In certain embodiments, the substitution, deletion, or insertion includes fewer than 25 amino acid substitutions, fewer than 20 amino acid substitutions, fewer than 15 amino acid substitutions, fewer than 10 amino acid substitutions, fewer than 5 amino acid substitutions, fewer than 4 amino acid substitutions, fewer than 3 amino acid substitutions, or fewer than 2 amino acid substitutions relative to the original molecule. In a specific embodiment, the substitution is a conservative amino acid substitution made at one or more predicted non-essential amino acid residues. The variation allowed may be determined by systematically making insertions, deletions, or substitutions of amino acids in the sequence and testing the resulting variants for activity exhibited by the parental polypeptides.

The polypeptides generated by conservative amino acid substitutions are included in the present disclosure. Conservative (e.g., within an amino acid group with similar properties and/or side chains) substitutions may be made, so as to maintain or not significantly change the properties.

One type of substitutional variant involves substituting one or more hypervariable region residues of a parent antibody (e.g., a humanized or human antibody) or fragment thereof in the extraceullar antigen binding domain of the present CARs. Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more CDR residues are mutated and the variant antibodies displayed on phage and screened for a particular biological activity (e.g. binding affinity).

Alterations (e.g., substitutions) may be made in CDRs, e.g., to improve antibody affinity. Such alterations may be made in CDR “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant antibody or fragment thereof being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized. CDR residues involved in antigen binding may be specifically identified, e.g., using alanine scanning mutagenesis or modeling.

In some embodiments, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity may be made in CDRs. In some embodiments, each CDR either is unaltered, or contains no more than one, two or three amino acid substitutions.

A useful method for identification of residues or regions of an antibody that may be targeted for mutagenesis is called “alanine scanning mutagenesis” as described by Cunningham and Wells, Science, 244:1081-1085 (1989). Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

The variations can be made using methods known in the art such as oligonucleotide-mediated (site-directed) mutagenesis, alanine scanning, and PCR mutagenesis. Site-directed mutagenesis (see, e.g., Carter, Biochem J. 237:1-7 (1986); and Zoller et al., Nucl. Acids Res. 10:6487-500 (1982)), cassette mutagenesis (see, e.g., Wells et al., Gene 34:315-23 (1985)), or other known techniques can be performed on the cloned DNA to produce the antibody variant DNA.

Also included in the present disclosure are immune effector cells comprising two or more kinds of functional exogenous receptors, such dual CARs or tandem CARs that binds two different targets.

In some specific embodiments, the CAR provided herein binds to Glypican-3 (GPC3). GPC3, a 65-kDa protein of 580 amino acids, is a heparan sulfate proteoglycan anchored to the cell membrane by glycosylphosphatidylinositol. GPC3 is expressed in hepatocellular carcinoma (HCC), ovarian clear cell carcinoma (OCCC), melanomas, lung squamous cell carcinomas, hepatoblastomas, nephroblastomas (Wilms' tumors), and yolk sac tumors, as well as in certain stomach cancers, for example, gastric cancers that produce a-fetoprotein. The exact function of secreted and membrane-anchored GPC3 in these cancers is not entirely clear, but it is demonstrated to involve in neoplastic transformation, e.g., in HCC (Shirakawa H., et al, Cancer Sci. 2009; 100:1403-1407). Strikingly, the protein is nearly absent in all other cancer forms. GPC3 sequences are known in the art. In some embodiments, GPC3 comprises the amino acid sequence of at least one selected from GenBank Accession numbers: NM_001164617 and NP_001158089, NM_004484 and NP_004475, NM_001164618 and NP_001158090, and NM_001164619 and NP_001158091. In some specific embodiments, the CAR that binds to GPC3 provided herein comprises the amino acid sequence of SEQ ID NO: 29 or 135.

### 5.2.3. Immune Effector Cells

“Immune effector cells” are immune cells that can perform immune effector functions. In some embodiments, the immune effector cells express at least FcTRIII and perform ADCC effector function. Examples of immune effector cells which mediate ADCC include peripheral blood mononuclear cells (PBMC), natural killer (NK) cells, monocytes, cytotoxic T cells, neutrophils, and eosinophils.

In some embodiments, the immune effector cells are T cells. In some embodiments, the T cells are CD4⁺/CD8⁻, CD4⁻/CD8⁺, CD4⁺/CD8⁺, CD4⁻/CD8⁻, or combinations thereof. In some embodiments, the T cells produce IL-2, IFN, and/or TNF upon expressing the CAR and binding to the target cells. In some embodiments, the CD8⁺ T cells lyse antigen-specific target cells upon expressing the CAR and binding to the target cells.

In some embodiments, the immune effector cells are NK cells. In other embodiments, the immune effector cells can be established cell lines, for example, NK-92 cells.

In some embodiments, the immune effector cells are differentiated from a stem cell, such as a hematopoietic stem cell, a pluripotent stem cell, an iPS, or an embryonic stem cell.

The engineered immune effector cells are prepared by introducing the polypeptide provided herein into the immune effector cells, such as T cells. In some embodiments, the polypeptide is introduced to the immune effector cells by transfecting any one of the isolated nucleic acids or any one of the vectors described above.

Methods of introducing vectors or isolated nucleic acids into a mammalian cell are known in the art. The vectors described can be transferred into an immune effector cell by physical, chemical, or biological methods.

Physical methods for introducing the vector into an immune effector cell include calcium phosphate precipitation, lipofection, particle bombardment, microinjection, electroporation, and the like. Methods for producing cells comprising vectors and/or exogenous nucleic acids are well-known in the art. See, e.g., Sambrook et al. (2001) Molecular Cloning: A Laboratory Manual, Cold Spring Harbor Laboratory, New York. In some embodiments, the vector is introduced into the cell by electroporation.

Biological methods for introducing the vector into an immune effector cell include the use of DNA and RNA vectors. Viral vectors have become the most widely used method for inserting genes into mammalian, e.g., human cells.

Chemical means for introducing the vector into an immune effector cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro is a liposome (e.g., an artificial membrane vesicle).

In some embodiments, RNA molecules encoding any of the polypeptides described herein may be prepared by a conventional method (e.g., in vitro transcription) and then introduced into the immune effector cells via known methods such as mRNA electroporation. See, e.g., Rabinovich et al., Human Gene Therapy 17:1027-1035 (2006).

In some embodiments, the transduced or transfected immune effector cell is propagated ex vivo after introduction of the vector or isolated nucleic acid. In some embodiments, the transduced or transfected immune effector cell is cultured to propagate for at least about any of 1 day, 2 days, 3 days, 4 days, 5 days, 6 days, 7 days, 10 days, 12 days, or 14 days. In some embodiments, the transduced or transfected immune effector cell is further evaluated or screened to select the engineered mammalian cell.

Reporter genes may be used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al. FEBS Letters 479: 79-82 (2000)). Suitable expression systems are well known and may be prepared using known techniques or obtained commercially.

Other methods to confirm the presence of the nucleic acid encoding the polypeptide in the engineered immune effector cell, include, for example, molecular biological assays well known to those of skill in the art, such as Southern and Northern blotting, RT-PCR and PCR; biochemical assays, such as detecting the presence or absence of a particular peptide, e.g., by immunological methods (such as ELISAs and Western blots).

### 5.2.4. Sources of T Cells

In some embodiments, prior to expansion and genetic modification of the T cells, a source of T cells is obtained from a subject. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In some embodiments, any number of T cell lines available in the art, may be used. In some embodiments, T cells can be obtained from a unit of blood collected from a subject using any number of techniques known to the skilled artisan, such as Ficoll™ separation. In some embodiments, cells from the circulating blood of an individual are obtained by apheresis. The apheresis product typically contains lymphocytes, including T cells, monocytes, granulocytes, B cells, other nucleated white blood cells, red blood cells, and platelets. In some embodiments, the cells collected by apheresis may be washed to remove the plasma fraction and to place the cells in an appropriate buffer or media for subsequent processing steps. In some embodiments, the cells are washed with phosphate buffered saline (PBS). In some embodiments, the wash solution lacks calcium and may lack magnesium or may lack many if not all divalent cations. Initial activation steps in the absence of calcium may lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca²⁺-free, Mg²⁺-free PBS, PlasmaLyte A, or other saline solution with or without buffer. Alternatively, the undesirable components of the apheresis sample may be removed and the cells directly resuspended in culture media.

In some embodiments, T cells are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3⁺, CD28⁺, CD4⁺, CD8⁺, CD45RA⁺, and CD45RO⁺T cells, can be further isolated by positive or negative selection techniques. For example, in some embodiments, T cells are isolated by incubation with anti-CD3/anti-CD28 (i.e., 3×28)-conjugated beads, such as DYNABEADS© M-450 CD3/CD28 T, for a time period sufficient for positive selection of the desired T cells. In some embodiments, the time period is about 30 minutes. In a further embodiment, the time period ranges from 30 minutes to 36 hours or longer and all integer values there between. In a further embodiment, the time period is at least 1, 2, 3, 4, 5, or 6 hours. In some embodiments, the time period is 10 to 24 hours. In some embodiments, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumor infiltrating lymphocytes (TIL) from tumor tissue or from immune-compromised individuals. Further, use of longer incubation times can increase the efficiency of capture of CD8⁺ T cells. Thus, in some embodiments, by simply shortening or lengthening the time T cells are allowed to bind to the CD3/CD28 beads and/or by increasing or decreasing the ratio of beads to T cells, subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used. In some embodiments, it may be desirable to perform the selection procedure and use the “unselected” cells in the activation and expansion process. “Unselected” cells can also be subjected to further rounds of selection.

Enrichment of a T cell population by negative selection can be accomplished with a combination of antibodies directed to surface markers unique to the negatively selected cells. One method is cell sorting and/or selection via negative magnetic immuno adherence or flow cytometry that uses a cocktail of monoclonal antibodies directed to cell surface markers present on the cells negatively selected. For example, to enrich for CD4⁺ cells by negative selection, a monoclonal antibody cocktail typically includes antibodies to CD14, CD20, CD11b, CD16, HLA-DR, and CD8. In certain embodiments, it may be desirable to enrich for or positively select for regulatory T cells which typically express CD4⁺, CD25⁺, CD62Lhi, GITR⁺, and FoxP3⁺. Alternatively, in certain embodiments, T regulatory cells are depleted by anti-C25 conjugated beads or other similar method of selection.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/mL is used. In one embodiment, a concentration of 1 billion cells/mL is used. In a further embodiment, greater than 100 million cells/mL is used. In a further embodiment, a concentration of cells of 10, 15, 20, 25, 30, 35, 40, 45, or 50 million cells/mL is used. In yet another embodiment, a concentration of cells from 75, 80, 85, 90, 95, or 100 million cells/mL is used. In further embodiments, concentrations of 125 or 150 million cells/mL can be used. Using high concentrations may result in increased cell yield, cell activation, and cell expansion. Further, use of high cell concentrations may allow more efficient capture of cells that may weakly express target antigens of interest, such as CD28-negative T cells, or from samples where there are many tumor cells present (i.e., leukemic blood, tumor tissue, etc.). Such populations of cells may have therapeutic value and would be desirable to obtain. In some embodiments, using high concentration of cells allows more efficient selection of CD8+ T cells that normally have weaker CD28 expression.

In some embodiments, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4⁺ T cells express higher levels of CD28 and are more efficiently captured than CD8+ T cells in dilute concentrations. In some embodiments, the concentration of cells used is 5×10⁶/mL. In some embodiments, the concentration used can be from about 1×10⁵/mL to 1×10⁶/mL, and any integer value in between.

In some embodiments, the cells may be incubated on a rotator for varying lengths of time at varying speeds at either 2-10° C., or at room temperature.

T cells for stimulation can also be frozen after a washing step. Without being bound by theory, the freeze and subsequent thaw step may provide a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% dextran 40 and 5% dextrose, 20% human serum albumin and 7.5% DMSO, or 31.25% plasmalyte-A, 31.25% dextrose 5%, 0.45% NaCl, 10% dextran 40 and 5% dextrose, 20% human serum albumin, and 7.5% DMSO or other suitable cell freezing media containing for example, Hespan and PlasmaLyte A. The cells then are frozen to −80° C. at a rate of 1° C. per minute and stored in the vapor phase of a liquid nitrogen storage tank. Other methods of controlled freezing may be used as well as uncontrolled freezing immediately at −20° C. or in liquid nitrogen.

In some embodiments, cryopreserved cells are thawed and washed as described herein and allowed to rest for one hour at room temperature prior to activation.

Also contemplated in the present disclosure is the collection of blood samples or apheresis product from a subject at a time period prior to when the expanded cells as described herein might be needed. As such, the source of the cells to be expanded can be collected at any time point necessary, and desired cells, such as T cells, isolated and frozen for later use in T cell therapy for any number of diseases or conditions that would benefit from T cell therapy, such as those described herein. In one embodiment, a blood sample or an apheresis is taken from a generally healthy subject. In certain embodiments, a blood sample or an apheresis is taken from a generally healthy subject who is at risk of developing a disease, but who has not yet developed a disease, and the cells of interest are isolated and frozen for later use. In certain embodiments, the T cells may be expanded, frozen, and used at a later time. In certain embodiments, samples are collected from a patient shortly after diagnosis of a particular disease as described herein but prior to any treatments. In a further embodiment, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, cytoxan, fludarabine, cyclosporin, FK506, rapamycin, mycophenolic acid, steroids, FR901228, and irradiation. These drugs inhibit either the calcium dependent phosphatase calcineurin (cyclosporine and FK506) or inhibit the p70S6 kinase that is important for growth factor induced signaling (rapamycin) (Liu et al., Cell 66:807-815 (1991); Henderson et al., Immun 73:316-321 (1991); Bierer et al., Curr. Opin. Immun. 5:763-773 (1993)). In a further embodiment, the cells are isolated for a patient and frozen for later use in conjunction with (e.g., before, simultaneously or following) bone marrow or stem cell transplantation, T cell ablative therapy using either chemotherapy agents such as, fludarabine, external-beam radiation therapy (XRT), cyclophosphamide, or antibodies such as OKT3 or CAMPATH. In another embodiment, the cells are isolated prior to and can be frozen for later use for treatment following B-cell ablative therapy such as agents that react with CD20, e.g., Rituxan.

In some embodiments, T cells are obtained from a patient directly following treatment. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present disclosure to collect blood cells, including T cells, dendritic cells, or other cells of the hematopoietic lineage, during this recovery phase. Further, in certain embodiments, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system. 5.2.5. Activation and Expansion of T Cells

In some embodiments, prior to or after genetic modification of the T cells with the CARs described herein, the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

Generally, T cells can be expanded by contact with a surface having attached thereto an agent that stimulates a CD3/TCR complex associated signal and a ligand that stimulates a co-stimulatory molecule on the surface of the T cells. In particular, T cell populations may be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4⁺ T cells or CD8⁺ T cells, an anti-CD3 antibody and an anti-CD28 antibody. Examples of an anti-CD3 antibody include UCHT1, OKT3, HIT3a (BioLegend, San Diego, US) can be used as can other methods commonly known in the art (Graves J, et al., J. Immunol. 146:2102 (1991); Li B, et al., Immunology 116:487 (2005); Rivollier A, et al., Blood 104:4029 (2004)). Examples of an anti-CD28 antibody include 9.3, B-T3, XR-CD28 (Diaclone, Besancon, France) can be used as can other methods commonly known in the art (Berg et al., Transplant Proc. 30(8):3975-3977 (1998); Haanen et al., J. Exp. Med. 190(9):13191328 (1999); Garland et al., J. Immunol Meth. 227(1-2):53-63 (1999)).

In some embodiments, the primary stimulatory signal and the co-stimulatory signal for the T cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In one embodiment, the agent providing the co-stimulatory signal is bound to a cell surface and the agent providing the primary activation signal is in solution or coupled to a surface. In certain embodiments, both agents can be in solution. In another embodiment, the agents may be in soluble form, and then cross-linked to a surface, such as a cell expressing Fc receptors or an antibody or other binding agent which will bind to the agents. In this regard, see for example, U.S. Patent Application Publication Nos. 20040101519 and 20060034810 for artificial antigen presenting cells (aAPCs) that are contemplated for use in activating and expanding T cells in certain embodiments in the present disclosure.

In some embodiments, the T cells, are combined with agent-coated beads, the beads and the cells are subsequently separated, and then the cells are cultured. In an alternative embodiment, prior to culture, the agent-coated beads and cells are not separated but are cultured together. In a further embodiment, the beads and cells are first concentrated by application of a force, such as a magnetic force, resulting in increased ligation of cell surface markers, thereby inducing cell stimulation.

By way of example, cell surface proteins may be ligated by allowing paramagnetic beads to which anti-CD3 and anti-CD28 are attached (3×28 beads) to contact the T cells. In one embodiment, the cells (for example, 10⁴ to 4×10⁸ T cells) and beads (for example, anti-CD3/CD28 MACSiBead particles at a recommended titer of 1:100) are combined in a buffer, preferably PBS (without divalent cations such as, calcium and magnesium). Those of ordinary skill in the art can readily appreciate any cell concentration may be used. For example, the target cell may be very rare in the sample and comprise only 0.01% of the sample or the entire sample (i.e., 100%) may comprise the target cell of interest. Accordingly, any cell number is within the context of the present disclosure. In certain embodiments, it may be desirable to significantly decrease the volume in which particles and cells are mixed together (i.e., increase the concentration of cells), to ensure maximum contact of cells and particles. For example, in one embodiment, a concentration of about 2 billion cells/mL is used. In another embodiment, greater than 100 million cells/mL is used. In a further embodiment, a concentration of cells of 10, 15, 20, 25, 30, 35, 40, 45, or 50 million cells/mL is used. In yet another embodiment, a concentration of cells from 75, 80, 85, 90, 95, or 100 million cells/mL is used. In further embodiments, concentrations of 125 or 150 million cells/mL can be used. Using high concentrations may result in increased cell yield, cell activation, and cell expansion. Further, use of high cell concentrations may allow more efficient capture of cells that may weakly express target antigens of interest, such as CD28-negative T cells. Such populations of cells may have therapeutic value and would be desirable to obtain in certain embodiments. For example, using high concentration of cells allows more efficient selection of CD8+ T cells that normally have weaker CD28 expression.

In some embodiments, the mixture may be cultured for several hours (about 3 hours) to about 14 days or any hourly integer value in between. In another embodiment, the mixture may be cultured for 21 days. In one embodiment, the beads and the T cells are cultured together for about eight days. In another embodiment, the beads and T cells are cultured together for 2-3 days. Several cycles of stimulation may also be desired such that culture time of T cells can be 60 days or more. Conditions appropriate for T cell culture include an appropriate media (e.g., Minimal Essential Media or RPMI Media 1640 or X-vivo 15 (Lonza)) that may contain factors necessary for proliferation and viability, including serum (e.g., fetal bovine or human serum), interleukin-2 (IL-2), insulin, IFN-γ, IL-4, IL-7, GM-CSF, IL-10, IL-12, IL-15, TGFβ, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, X-Vivo 20, optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂). T cells that have been exposed to varied stimulation times may exhibit different characteristics. For example, typical blood or apheresed peripheral blood mononuclear cell products have a helper T cell population (TH, CD4⁺) that is greater than the cytotoxic or suppressor T cell population (TC, CD8). Ex vivo expansion of T cells by stimulating CD3 and CD28 receptors produces a population of T cells that prior to about days 8-9 consists predominately of TH cells, while after about days 8-9, the population of T cells comprises an increasingly greater population of TC cells. Accordingly, depending on the purpose of treatment, infusing a subject with a T cell population comprising predominately of TH cells may be advantageous. Similarly, if an antigen-specific subset of TC cells has been isolated it may be beneficial to expand this subset to a greater degree.

Further, in addition to CD4 and CD8 markers, other phenotypic markers vary significantly, but in large part, reproducibly during the course of the cell expansion process. Thus, such reproducibility enables the ability to tailor an activated T cell product for specific purposes. 5.3. Polypeptides Comprising a Functional Exogenous Receptor and Chimeric Cytokine Receptor

In another aspect, provided herein is a polypeptide comprising at least one functional exogenous receptor (such as CAR, TCR and TAC) and a chimeric cytokine receptor provided herein. The chimeric cytokine receptor in the present polypeptide can be any of those described in Section 5.2.1 above. The functional exogenous receptor in the present polypeptide can be any of those described in Section 5.2.2 above.

In the present polypeptides, the chimeric cytokine receptor may be at the N-terminus of the functional exogenous receptor, or the chimeric cytokine receptor may be at the C-terminus of the functional exogenous receptor.

In some embodiments of the various polypeptides provided herein, the chimeric cytokine receptor and the functional exogenous receptor are linked with each other via a peptide linker. In some embodiments, the peptide linker is a self-cleaving peptide such as 2A self-cleaving peptide, so that the chimeric cytokine receptor and the functional exogenous receptor become separate polypeptides upon cleavage in cells.

The members of 2A peptides are named after the virus in which they have been first described. For example, F2A, the first described 2A peptide, is derived from foot-and-mouth disease virus. The self-cleaving 18-22 amino acids long 2A peptides mediate ‘ribosomal skipping’ between the proline and glycine residues and inhibit peptide bond formation without affecting downstream translation. These peptides allow multiple proteins to be encoded as polyproteins, which dissociate into component proteins upon translation. Self-cleaving peptides are found in members of the picornaviridae virus family, including aphthoviruses such as foot-and-mouth disease virus (FMDV), equine rhinitis A virus (ERAV), thosea asigna virus (TaV) and porcine teschovirus-1 (PTV-1) (see Donnelly et al., J. Gen. Virol., 82: 1027-101 (2001); Ryan et al., J. Gen. Virol., 72: 2727-2732 (2001)) and cardioviruses such as theilovirus (e.g., theiler's murine encephalomyelitis) and encephalomyocarditis viruses. The 2A peptides derived from FMDV, ERAV, PTV-1, and TaV are sometimes referred to as “F2A,” “E2A,” “P2A,” and “T2A,” respectively, and are included in the present disclosure, e.g., as described in Donnelly et al., J. Gen. Virol., 78: 13-21 (1997); Ryan and Drew, EMBO J., 13: 928-933 (1994); Szymczak et al., Nature Biotech., 5: 589-594 (2004); Hasegawa et al., Stem Cells, 25(7): 1707-12 (2007). In yet other embodiments, intein mediated protein splicing system is used herein, e.g., as described in Shah and Muir, Chem Sci., 5(1): 446-461 (2014) and Topilina and Mills, Mobile DNA, 5 (5) (2014). Other methods known in the art can also be used in the present constructs.

In some embodiments, the 2A self-cleaving peptide is selected from a group consisting of F2A, E2A, P2A, T2A, or variants thereof. In some embodiments, the self-cleaving peptide is a 2A self-cleaving peptide P2A fragment. In a specific embodiment, the self-cleaving peptide is T2A fragment.

In another aspect, provided herein is a nucleic acid encoding a polypeptide described above comprising a functional exogenous receptor (such as a CAR) provided herein and a functional exogenous receptor, which is described in more detail below.

### 5.4. Polynucleotides

In another aspect, the disclosure provides polynucleotides that encode the polypeptide provided herein, including those described in Section 5.2 and Section 5.3 above.

More specifically, in some embodiments, provided herein is a polynucleotide encoding a polypeptide comprising a chimeric cytokine receptor and a functional exogenous receptor (suchy as a CAR), wherein the chimeric cytokine receptor and the functional exogenous receptor are linked with each other by peptide linkers, such as 2A self-cleaving peptide linkers.

In yet another aspect, provided herein is a polynucleotide comprising a region encoding a chimeric cytokine receptor provided herein (e.g., as those described in Section 5.2.1) and a region encoding a functional exogenous receptor provided herein (e.g., as those described in Section 5.2.2). In some embodiments, the two regions are controlled by the same promoter. For example, in some embodiments, internal ribosomal entry sites (IRES) are used herein to express multiple genes from one promoter. In other embodiments, these regions are controlled by separate promoters.

In yet another aspect, provided herein is a composition comprising a first polynucleotide encoding a chimeric cytokine receptor provided herein (e.g., as those described in Section 5.2.1), a second polynucleotide encoding a functional exogenous receptor provided herein (e.g., as those described in Section 5.2.2).

The polynucleotides of the disclosure can be in the form of RNA or in the form of DNA. DNA includes cDNA, genomic DNA, and synthetic DNA; and can be double-stranded or single-stranded, and if single stranded can be the coding strand or non-coding (anti-sense) strand. In some embodiments, the polynucleotide is in the form of cDNA. In some embodiments, the polynucleotide is a synthetic polynucleotide.

The present disclosure further relates to variants of the polynucleotides described herein, wherein the variant encodes, for example, fragments, analogs, and/or derivatives of the polypeptide of the disclosure. In certain embodiments, the present disclosure provides a polynucleotide comprising a polynucleotide having a nucleotide sequence at least about 75% identical, at least about 80% identical, at least about 85% identical, at least about 90% identical, at least about 95% identical, and in some embodiments, at least about 96%, 97%, 98% or 99% identical to a polynucleotide encoding the polypeptide of the disclosure. As used herein, the phrase “a polynucleotide having a nucleotide sequence at least, for example, 95% “identical” to a reference nucleotide sequence” is intended to mean that the nucleotide sequence of the polynucleotide is identical to the reference sequence except that the polynucleotide sequence can include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. These mutations of the reference sequence can occur at the 5′ or 3′ terminal positions of the reference nucleotide sequence or anywhere between those terminal positions, interspersed either individually among nucleotides in the reference sequence or in one or more contiguous groups within the reference sequence.

The polynucleotide variants can contain alterations in the coding regions, non-coding regions, or both. In some embodiments, a polynucleotide variant contains alterations which produce silent substitutions, additions, or deletions, but does not alter the properties or activities of the encoded polypeptide. In some embodiments, a polynucleotide variant comprises silent substitutions that results in no change to the amino acid sequence of the polypeptide (due to the degeneracy of the genetic code). Polynucleotide variants can be produced for a variety of reasons, for example, to optimize codon expression for a particular host (i.e., change codons in the human mRNA to those preferred by a bacterial host such as E. coli). In some embodiments, a polynucleotide variant comprises at least one silent mutation in a non-coding or a coding region of the sequence.

In some embodiments, a polynucleotide variant is produced to modulate or alter expression (or expression levels) of the encoded polypeptide. In some embodiments, a polynucleotide variant is produced to increase expression of the encoded polypeptide. In some embodiments, a polynucleotide variant is produced to decrease expression of the encoded polypeptide. In some embodiments, a polynucleotide variant has increased expression of the encoded polypeptide as compared to a parental polynucleotide sequence. In some embodiments, a polynucleotide variant has decreased expression of the encoded polypeptide as compared to a parental polynucleotide sequence.

### 5.5. Vectors

Also provided are vectors comprising the polynucleotides or nucleic acid molecules described herein. In one embodiment, the nucleic acid molecules can be incorporated into a recombinant expression vector.

The present disclosure provides vectors for cloning and expressing any one of the polypeptides described herein. In some embodiments, the vector is suitable for replication and integration in eukaryotic cells, such as mammalian cells. In some embodiments, the vector is a viral vector. Examples of viral vectors include, but are not limited to, adenoviral vectors, adeno-associated virus vectors, lentiviral vector, retroviral vectors, vaccinia vector, herpes simplex viral vector, and derivatives thereof. Viral vector technology is well known in the art and is described, for example, in Sambrook et al. (2001, Molecular Cloning: A Laboratory Manual, Cold Spring Harbor Laboratory, New York), and in other virology and molecular biology manuals.

A number of viral based systems have been developed for gene transfer into mammalian cells. For example, retroviruses provide a convenient platform for gene delivery systems. The heterologous nucleic acid can be inserted into a vector and packaged in retroviral particles using techniques known in the art. The recombinant virus can then be isolated and delivered to the engineered mammalian cell in vitro or ex vivo. A number of retroviral systems are known in the art. In some embodiments, adenovirus vectors are used. A number of adenovirus vectors are known in the art. In some embodiments, lentivirus vectors are used. In some embodiments, self-inactivating lentiviral vectors are used. For example, self-inactivating lentiviral vectors carrying the immunomodulator (such as immune checkpoint inhibitor) coding sequence and/or self-inactivating lentiviral vectors carrying chimeric antigen receptors can be packaged with protocols known in the art. The resulting lentiviral vectors can be used to transduce a mammalian cell (such as primary human T cells) using methods known in the art. Vectors derived from retroviruses such as lentivirus are suitable tools to achieve long-term gene transfer, because they allow long-term, stable integration of a transgene and its propagation in progeny cells. Lentiviral vectors also have low immunogenicity, and can transduce non-proliferating cells.

In some embodiments, the vector comprises any one of the nucleic acids encoding a polypeptide described herein. The nucleic acid can be cloned into the vector using any known molecular cloning methods in the art, including, for example, using restriction endonuclease sites and one or more selectable markers. In some embodiments, the nucleic acid is operably linked to a promoter. Varieties of promoters have been explored for gene expression in mammalian cells, and any of the promoters known in the art may be used in the present disclosure. Promoters may be roughly categorized as constitutive promoters or regulated promoters, such as inducible promoters.

In some embodiments, the nucleic acid encoding the polypeptide is operably linked to a constitutive promoter. Constitutive promoters allow heterologous genes (also referred to as transgenes) to be expressed constitutively in the host cells. Exemplary constitutive promoters contemplated herein include, but are not limited to, murine stem cell virus (MSCV) promoter, cytomegalovirus (CMV) promoters, human elongation factors-1 alpha (hEFla), ubiquitin C promoter (UbiC), phosphoglycerokinase promoter (PGK), simian virus 40 early promoter (SV40), and chicken β-Actin promoter coupled with CMV early enhancer (CAGG). The efficiencies of such constitutive promoters on driving transgene expression have been widely compared in a huge number of studies. For example, Michael C. Milone et al compared the efficiencies of CMV, hEFla, UbiC and PGK to drive chimeric antigen receptor expression in primary human T cells, and concluded that hEFla promoter not only induced the highest level of transgene expression, but was also optimally maintained in the CD4 and CD8 human T cells (Molecular Therapy, 17(8): 1453-1464 (2009)). In some embodiments, the nucleic acid encoding the CAR is operably linked to a hEF1α promoter. In some embodiments, the nucleic acid encoding the CAR is operably linked to a MSCV promoter.

In some embodiments, the nucleic acid encoding the polypeptide is operably linked to an inducible promoter. Inducible promoters belong to the category of regulated promoters. The inducible promoter can be induced by one or more conditions, such as a physical condition, microenvironment of the engineered immune effector cell, or the physiological state of the engineered immune effector cell, an inducer (i.e., an inducing agent), or a combination thereof.

In some embodiments, the inducing condition does not induce the expression of endogenous genes in the engineered mammalian cell, and/or in the subject that receives the pharmaceutical composition. In some embodiments, the inducing condition is selected from the group consisting of: inducer, irradiation (such as ionizing radiation, light), temperature (such as heat), redox state, tumor environment, and the activation state of the engineered mammalian cell.

In some embodiments, the vector also contains a selectable marker gene or a reporter gene to select cells expressing the polypeptide from the population of host cells transfected through lentiviral vectors. Both selectable markers and reporter genes may be flanked by appropriate regulatory sequences to enable expression in the host cells. For example, the vector may contain transcription and translation terminators, initiation sequences, and promoters useful for regulation of the expression of the nucleic acid sequences.

### 5.6. Methods of Making

In yet another aspect, provided herein is a method for making an engineered immune effector cell (e.g., a CAR-T cell or a TCR-T cell or a TAC-T cell), comprising introducing one or more polynucleotide(s) or the vector(s) provided herein (e.g., as described in Section 5.4 and Section 5.5 above) into an immune effector cell (e.g., a T cell).

More specifically, in some embodiments, engineered immune effector cells provided herein can be produced by introducing one or more nucleic acid(s) encoding a polypeptide described in Section 5.3 or one or more nucleic acid(s) described in Section 5.4 into T cells.

The chimeric cytokine receptor and the functional exogenous receptor can each be introduced into T cells separately as separately polypeptides.

Alternatively, engineered immune effector cells provided herein can be produced by a polynucleotide comprising multiple regions, for example, a region encoding a CAR, a region encoding a chimeric cytokine receptor. Different regions can be controlled by the same promoter. For example, in some embodiments, internal ribosomal entry sites (IRES) are used herein to express multiple genes from one promoter. In other embodiments, different regions are controlled by separate promoters.

In yet another aspect, provided herein is an engineered immune effector cell (e.g., a CAR-T cell) produced according to the method provided herein.

The methods described above in the context of CAR-T cells are also applicable to production of other immune effector cells such as TCR-T cells and TAC-T cells.

### 5.7. Pharmaceutical Compositions

In one aspect, the present disclosure further provides pharmaceutical compositions comprising an engineered cell of the present disclosure. In some embodiments, a pharmaceutical composition comprises a therapeutically effective amount of the engineered T cell of the present disclosure and a pharmaceutically acceptable excipient.

In a specific embodiment, the term “excipient” can also refer to a diluent, adjuvant (e.g., Freunds' adjuvant (complete or incomplete), carrier or vehicle. Pharmaceutical excipients can be sterile liquids, such as water and oils, including those of petroleum, animal, vegetable or synthetic origin, such as peanut oil, soybean oil, mineral oil, sesame oil and the like. Saline solutions and aqueous dextrose and glycerol solutions can also be employed as liquid excipients. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Examples of suitable pharmaceutical excipients are described in Remington's Pharmaceutical Sciences (1990) Mack Publishing Co., Easton, PA. Such compositions will contain a prophylactically or therapeutically effective amount of the active ingredient provided herein, such as in purified form, together with a suitable amount of excipient so as to provide the form for proper administration to the patient. The formulation should suit the mode of administration.

In some embodiments, the choice of excipient is determined in part by the particular cell, and/or by the method of administration. Accordingly, there are a variety of suitable formulations.

Typically, acceptable carriers, excipients, or stabilizers are nontoxic to recipients at the dosages and concentrations employed, and include buffers, antioxidants including ascorbic acid, methionine, Vitamin E, sodium metabisulfite; preservatives, isotonicifiers, stabilizers, metal complexes (e.g. Zn-protein complexes); chelating agents such as EDTA and/or non-ionic surfactants.

Buffers may be used to control the pH in a range which optimizes the therapeutic effectiveness, especially if stability is pH dependent. Suitable buffering agents for use with the present disclosure include both organic and inorganic acids and salts thereof. For example, citrate, phosphate, succinate, tartrate, fumarate, gluconate, oxalate, lactate, acetate. Additionally, buffers may comprise histidine and trimethylamine salts such as Tris.

Preservatives may be added to retard microbial growth. Suitable preservatives for use with the present disclosure include octadecyldimethylbenzyl ammonium chloride; hexamethonium chloride; benzalkonium halides (e.g., chloride, bromide, iodide), benzethonium chloride; thimerosal, phenol, butyl or benzyl alcohol; alkyl parabens such as methyl or propyl paraben; catechol; resorcinol; cyclohexanol, 3-pentanol, and m-cresol.

Tonicity agents, sometimes known as “stabilizers” can be present to adjust or maintain the tonicity of liquid in a composition. When used with large, charged biomolecules such as proteins and antibodies, they are often termed “stabilizers” because they can interact with the charged groups of the amino acid side chains, thereby lessening the potential for inter and intra-molecular interactions. Exemplary tonicity agents include polyhydric sugar alcohols, trihydric or higher sugar alcohols, such as glycerin, erythritol, arabitol, xylitol, sorbitol and mannitol.

Additional exemplary excipients include: (1) bulking agents, (2) solubility enhancers, (3) stabilizers and (4) agents preventing denaturation or adherence to the container wall. Such excipients include: polyhydric sugar alcohols (enumerated above); amino acids such as alanine, glycine, glutamine, asparagine, histidine, arginine, lysine, ornithine, leucine, 2-phenylalanine, glutamic acid, threonine, etc.; organic sugars or sugar alcohols such as sucrose, lactose, lactitol, trehalose, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, a-monothioglycerol and sodium thio sulfate; low molecular weight proteins such as human serum albumin, bovine serum albumin, gelatin or other immunoglobulins; hydrophilic polymers such as polyvinylpyrrolidone; monosaccharides (e.g., xylose, mannose, fructose, glucose; disaccharides (e.g., lactose, maltose, sucrose); trisaccharides such as raffinose; and polysaccharides such as dextrin or dextran.

Non-ionic surfactants or detergents (also known as “wetting agents”) may be present to help solubilize the therapeutic agent as well as to protect the therapeutic protein against agitation-induced aggregation, which also permits the formulation to be exposed to shear surface stress without causing denaturation of the active therapeutic protein or antibody. Suitable non-ionic surfactants include, e.g., polysorbates (20, 40, 60, 65, 80, etc.), polyoxamers (184, 188, etc.), PLURONIC® polyols, TRITON®, polyoxyethylene sorbitan monoethers (TWEEN®-20, TWEEN®-80, etc.), lauromacrogol 400, polyoxyl 40 stearate, polyoxyethylene hydrogenated castor oil 10, 50 and 60, glycerol monostearate, sucrose fatty acid ester, methyl celluose and carboxymethyl cellulose. Anionic detergents that can be used include sodium lauryl sulfate, dioctyle sodium sulfosuccinate and dioctyl sodium sulfonate. Cationic detergents include benzalkonium chloride or benzethonium chloride.

The route of administration is in accordance with known and accepted methods, such as by single or multiple bolus or infusion over a long period of time in a suitable manner, e.g., injection or infusion by subcutaneous, intravenous, intraperitoneal, intramuscular, intraarterial, intralesional or intraarticular routes, topical administration, inhalation or by sustained release or extended-release means.

In another embodiment, a pharmaceutical composition can be provided as a controlled release or sustained release system. In one embodiment, a pump may be used to achieve controlled or sustained release (see, e.g., Sefton, Crit. Ref Biomed. Eng. 14:201-40 (1987); Buchwald et al., Surgery 88:507-16 (1980); and Saudek et al., N. Engl. J. Med. 321:569-74 (1989)). In another embodiment, polymeric materials can be used to achieve controlled or sustained release of a prophylactic or therapeutic agent (e.g., a fusion protein as described herein) or a composition provided herein (see, e.g., Medical Applications of Controlled Release (Langer and Wise eds., 1974); Controlled Drug Bioavailability, Drug Product Design and Performance (Smolen and Ball eds., 1984); Ranger and Peppas, J. Macromol. Sci. Rev. Macromol. Chem. 23:61-126 (1983); Levy et al., Science 228:190-92 (1985); During et al., Ann. Neurol. 25:351-56 (1989); Howard et al., J. Neurosurg. 71:105-12 (1989); U.S. Pat. Nos. 5,679,377; 5,916,597; 5,912,015; 5,989,463; and 5,128,326; PCT Publication Nos. WO 99/15154 and WO 99/20253). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In one embodiment, the polymer used in a sustained release formulation is inert, free of leachable impurities, stable on storage, sterile, and biodegradable. In yet another embodiment, a controlled or sustained release system can be placed in proximity of a particular target tissue, for example, the nasal passages or lungs, thus requiring only a fraction of the systemic dose (see, e.g., Goodson, Medical Applications of Controlled Release Vol. 2, 115-38 (1984)). Controlled release systems are discussed, for example, by Langer, Science 249:1527-33 (1990). Any technique known to one of skill in the art can be used to produce sustained release formulations comprising one or more agents as described herein (see, e.g., U.S. Pat. No. 4,526,938, PCT publication Nos. WO 91/05548 and WO 96/20698, Ning et al., Radiotherapy & Oncology 39:179-89 (1996); Song et al., PDA J. of Pharma. Sci. & Tech. 50:372-97 (1995); Cleek et al., Pro. Int'l. Symp. Control. Rel. Bioact. Mater. 24:853-54 (1997); and Lam et al., Proc. Int'l. Symp. Control Rel. Bioact. Mater. 24:759-60 (1997)).

The pharmaceutical compositions described herein may also contain more than one active compound or agent as necessary for the particular indication being treated. Alternatively, or in addition, the composition may comprise a cytotoxic agent, chemotherapeutic agent, cytokine, immunosuppressive agent, or growth inhibitory agent. Such molecules are suitably present in combination in amounts that are effective for the purpose intended.

The active ingredients may also be entrapped in microcapsules prepared, for example, by coascervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 18th edition.

Various compositions and delivery systems are known and can be used with the therapeutic agents provided herein, including, but not limited to, encapsulation in liposomes, microparticles, microcapsules, recombinant cells capable of expressing the single domain antibody or therapeutic molecule provided herein, construction of a nucleic acid as part of a retroviral or other vector, etc.

In some embodiments, the pharmaceutical composition provided herein contains the binding molecules and/or cells in amounts effective to treat or prevent the disease or disorder, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined.

### 5.8. Methods and Uses

The methods disclosed herein can be used for various therapeutic purposes. In one aspect, the disclosure provides methods for treating a cancer in a subject, methods of reducing the rate of the increase of volume of a tumor in a subject overtime, methods of reducing the risk of developing a metastasis, or methods of reducing the risk of developing an additional metastasis in a subject. In some embodiments, the treatment can halt, slow, retard, or inhibit progression of a cancer. In some embodiments, the treatment can result in the reduction of in the number, severity, and/or duration of one or more symptoms of the cancer in a subject.

As used herein, in some embodiments, the treatment provided herein delay development of a disease or disorder, e.g., defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease or disorder. For example, a late stage cancer, such as development of metastasis, may be delayed. In other embodiments, the method or the use provided herein prevents a disease or disorder.

In some embodiments, the present cell therapies comprise therapies comprising T cells expressing the various CARs, TCRs, cTCRs, TACs, TAC-like chimeric receptors, CARs and CCR(s), TCRs and CCR(s), cTCRs and CCR(s), TACs and CCR(s), TAC-like chimeric receptors and CCR(s), or combinations thereof, described herein. In some embodiments, the present CAR-T cell therapies are used for treating solid tumor cancer. In other embodiments, the present CAR-T cell therapies are used for treating blood cancer.

In some embodiments, the subject has GPC3, AFP, Claudin 18.2, CD19, CD20, CD22, BCMA, GD2, DLL3, MSLN, CD30, CLL1 or CD33-positive cancer. In some embodiments, the subject has liver cancer (e.g., hepatocellular carcinoma), glioma, lung cancer, colorectal cancer, head and neck cancer, stomach cancer, renal cancer, urothelial cancer, testis cancer, breast cancer, cervical cancer, endometrial cancer, and/or ovarian cancer. In some embodiments, the subject has squamous cell lung carcinoma, or solid tumor. In some embodiments, the subject has a CNS tumor, thyroid cancer, gastrointestinal cancer, skin cancer, sarcoma, urogenital cancer, and/or germ cell tumor. GPC3-related cancers can be found, e.g., in Moek, Kirsten L., et al., The American Journal of Pathology 188.9 (2018): 1973-1981, which is incorporated herein by reference in its entirety.

In other embodiments, the disease or disorder is an autoimmune and inflammatory disease. In other embodiments, the disease or disorder is an infectious disease. In some embodiments, the inflammatory or autoimmune disease is selected from the group consisting of arthritis, colitis, psoriasis, severe asthma, and moderate to severe Crohn's disease.

In some embodiments, the disease or disorder is a hematological cancer, such as leukemia, lymphoma, or myeloma. In some embodiments, the cancer is selected from a group consisting of Hodgkin's lymphoma, non-Hodgkin's lymphoma (NHL), cutaneous B-cell lymphoma, activated B-cell lymphoma, diffuse large B-cell lymphoma (DLBCL), mantle cell lymphoma (MCL), follicular center lymphoma, transformed lymphoma, lymphocytic lymphoma of intermediate differentiation, intermediate lymphocytic lymphoma (ILL), diffuse poorly differentiated lymphocytic lymphoma (PDL), centrocytic lymphoma, diffuse small-cleaved cell lymphoma (DSCCL), peripheral T-cell lymphomas (PTCL), cutaneous T-Cell lymphoma, mantle zone lymphoma, low grade follicular lymphoma, multiple myeloma (MM), chronic lymphocytic leukemia (CLL), diffuse large B-cell lymphoma (DLBCL), myelodysplastic syndrome (MDS), acute T cell leukemia, acute myeloid leukemia (AML), acute promyelocytic leukemia, acute myeloblastic leukemia, acute megakaryoblastic leukemia, precursor B acute lymphoblastic leukemia, precursor T acute lymphoblastic leukemia, Burkitt's leukemia (Burkitt's lymphoma), acute biphenotypic leukemia, chronic myeloid lymphoma, chronic myelogenous leukemia (CML), and chronic monocytic leukemia. In a specific embodiment, the disease or disorder is myelodysplastic syndromes (MDS). In another specific embodiment, the disease or disorder is acute myeloid leukemia (AML). In another specific embodiment, the disease or disorder is chronic lymphocytic leukemia (CLL). In yet another specific embodiment, the disease or disorder is multiple myeloma (MM).

In other embodiments, the disease or disorder is a solid tumor cancer. In some embodiments, the solid tumor cancer is selected from a group consisting of a carcinoma, an adenocarcinoma, an adrenocortical carcinoma, a colon adenocarcinoma, a colorectal adenocarcinoma, a colorectal carcinoma, a ductal cell carcinoma, a lung carcinoma, a thyroid carcinoma, a nasopharyngeal carcinoma, a melanoma, a non-melanoma skin carcinoma, a liver cancer and a lung cancer.

In some embodiments, the disease or disorder is caused by a pathogen. In some embodiments, the pathogen causes an infectious disease. In some embodiments, the pathogen is a bacteria. In some embodiments, the pathogen is a parasite. In some embodiments, the pathogen is a virus.

In other embodiments, the disease or disorder is an immune or autoimmune disorder. In some embodiments, the disease or disorder is an inflammatory disease. Inflammation plays a fundamental role in host defenses and the progression of immune-mediated diseases. The inflammatory response is initiated in response to injury (e.g., trauma, ischemia, and foreign particles) and infection (e.g., bacterial or viral infection) by a complex cascade of events, including chemical mediators (e.g., cytokines and prostaglandins) and inflammatory cells (e.g., leukocytes). The inflammatory response is characterized by increased blood flow, increased capillary permeability, and the influx of phagocytic cells. These events result in swelling, redness, warmth (altered heat patterns), and pus formation at the site of injury or infection.

Methods for administration of cells for adoptive cell therapy are known, as described, e.g., in US Patent Application Publication No. 2003/0170238; U.S. Pat. No. 4,690,915; Rosenberg, Nat Rev Clin Oncol. 8 (10):577-85 (2011); Themeli et al., Nat Biotechnol. 31(10): 928-933 (2013); Tsukahara et al., Biochem Biophys Res Commun 438(1): 84-9 (2013); and Davila et al., PLoS ONE 8(4): e61338 (2013). These methods may be used in connection with the methods and compositions provided herein.

In some embodiments, the cell therapy (e.g., adoptive T cell therapy) is carried out by autologous transfer, in which the cells are isolated and/or otherwise prepared from the subject who is to receive the cell therapy, or from a sample derived from such a subject. Thus, in some aspects, the cells are derived from a subject in need of a treatment and the cells, following isolation and processing are administered to the same subject. In other embodiments, the cell therapy (e.g., adoptive T cell therapy) is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

In some embodiments, the subject, to whom the cells, cell populations, or compositions are administered is a primate, such as a human. The subject can be male or female and can be any suitable age, including infant, juvenile, adolescent, adult, and geriatric subjects. In some embodiments, the subject is a validated animal model for disease, adoptive cell therapy, and/or for assessing toxic outcomes.

The composition provided herein can be administered by any suitable means, for example, by injection, e.g., intravenous or subcutaneous injections. In some embodiments, they are administered by parenteral, intrapulmonary, and intranasal, and, if desired for local treatment, intralesional administration.

The amount of a prophylactic or therapeutic agent provided herein that will be effective in the prevention and/or treatment of a disease or condition can be determined by standard clinical techniques. Effective doses may be extrapolated from dose-response curves derived from in vitro or animal model test systems. For the prevention or treatment of disease, the appropriate dosage of the binding molecule or cell may depend on the type of disease or disorder to be treated, the type of binding molecule, the severity and course of the disease or disorder, whether the therapeutic agent is administered for preventive or therapeutic purposes, previous therapy, the patient's clinical history and response to the agent, and the discretion of the attending physician. The compositions, molecules and cells are in some embodiments suitably administered to the patient at one time or over a series of treatments. Multiple doses may be administered intermittently. An initial higher loading dose, followed by one or more lower doses may be administered.

In the context of genetically engineered cells, in some embodiments, a subject may be administered the range of about one million to about 100 billion cells and/or that amount of cells per kilogram of body weight. In some embodiments, wherein the pharmaceutical composition comprises any one of the engineered immune cells described herein, the pharmaceutical composition is administered at a dosage of at least about any of 10⁴, 10⁵, 10⁶, 107, 10⁸, or 10⁹ cells/kg of body weight of the individual. Dosages may vary depending on attributes particular to the disease or disorder and/or patient and/or other treatments.

In some embodiments, the pharmaceutical composition is administered for a single time. In some embodiments, the pharmaceutical composition is administered for multiple times (such as any of 2, 3, 4, 5, 6, or more times). In some embodiments, the pharmaceutical composition is administered once or multiple times during a dosing cycle. A dosing cycle can be, e.g., 1, 2, 3, 4, 5 or more week(s), or 1, 2, 3, 4, 5, or more month(s). The optimal dosage and treatment regime for a particular patient can be determined by one skilled in the art of medicine by monitoring the patient for signs of disease and adjusting the treatment accordingly.

In some embodiments, the compositions provided herein are administered as part of a combination treatment, such as simultaneously with or sequentially with, in any order, another therapeutic intervention, such as another antibody or engineered cell or receptor or agent, such as a cytotoxic or therapeutic agent.

In certain embodiments, once the cells are administered to a mammal (e.g., a human), the biological activity of the engineered cell populations is measured by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells also can be measured by assaying expression and/or secretion of certain cytokines, such as CD107a, IFNγ, IL-2, and TNF. In some aspects the biological activity is measured by assessing clinical outcome, such as reduction in tumor burden or load. 5.9. Kits and Articles of Manufacture

Further provided are kits, unit dosages, and articles of manufacture comprising any of the engineered immune effector cells described herein. In some embodiments, a kit is provided which contains any one of the pharmaceutical compositions described herein and preferably provides instructions for its use.

The kits of the present application are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

The article of manufacture can comprise a container and a label or package insert on or associated with the container. Suitable containers include, for example, bottles, vials, syringes, etc. The containers may be formed from a variety of materials such as glass or plastic. Generally, the container holds a composition which is effective for treating a disease or disorder (such as cancer) described herein, and may have a sterile access port (for example the container may be an intravenous solution bag or a vial having a stopper pierceable by a hypodermic injection needle). The label or package insert indicates that the composition is used for treating the particular condition in an individual. The label or package insert will further comprise instructions for administering the composition to the individual. The label may indicate directions for reconstitution and/or use. The container holding the pharmaceutical composition may be a multi-use vial, which allows for repeat administrations (e.g. from 2-6 administrations) of the reconstituted formulation. Package insert refers to instructions customarily included in commercial packages of therapeutic products that contain information about the indications, usage, dosage, administration, contraindications and/or warnings concerning the use of such therapeutic products. Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kits or article of manufacture may include multiple unit doses of the pharmaceutical composition and instructions for use, packaged in quantities sufficient for storage and use in pharmacies, for example, hospital pharmacies and compounding pharmacies.

The disclosure is generally disclosed herein using affirmative language to describe the numerous embodiments. The disclosure also specifically includes embodiments in which particular subject matter is excluded, in full or in part, such as substances or materials, method steps and conditions, protocols, procedures, assays or analysis. Thus, even though the disclosure is generally not expressed herein in terms of what the disclosure does not include, aspects that are not expressly included in the disclosure are nevertheless disclosed herein.

A number of embodiments of the disclosure have been described. Nevertheless, it will be understood that various modifications may be made without departing from the spirit and scope of the disclosure. Accordingly, the following examples are intended to illustrate but not limit the scope of disclosure described in the claims.

### 6. EXAMPLES

The following is a description of various methods and materials used in the studies, and are put forth so as to provide those of ordinary skill in the art with a complete disclosure and description of how to make and use the present disclosure, and are not intended to limit the scope of what the inventors regard as their disclosure nor are they intended to represent that the experiments below were performed and are all of the experiments that may be performed. It is to be understood that exemplary descriptions written in the present tense were not necessarily performed, but rather that the descriptions can be performed to generate the data and the like associated with the teachings of the present disclosure. Efforts have been made to ensure accuracy with respect to numbers used (e.g., amounts, percentages, etc.), but some experimental errors and deviations should be accounted for.

### 6.1. Example 1—Plasmid Construction, Virus Preparation, Titer Evaluation

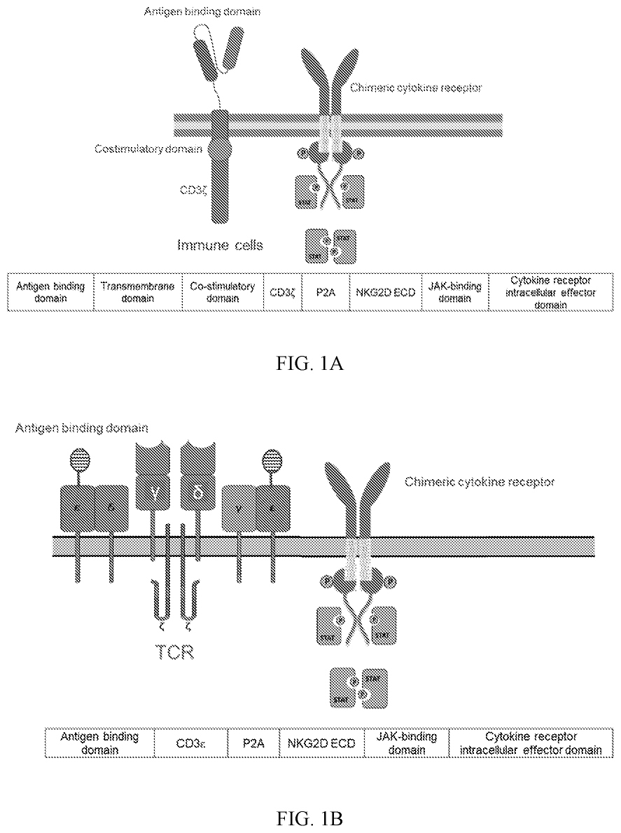

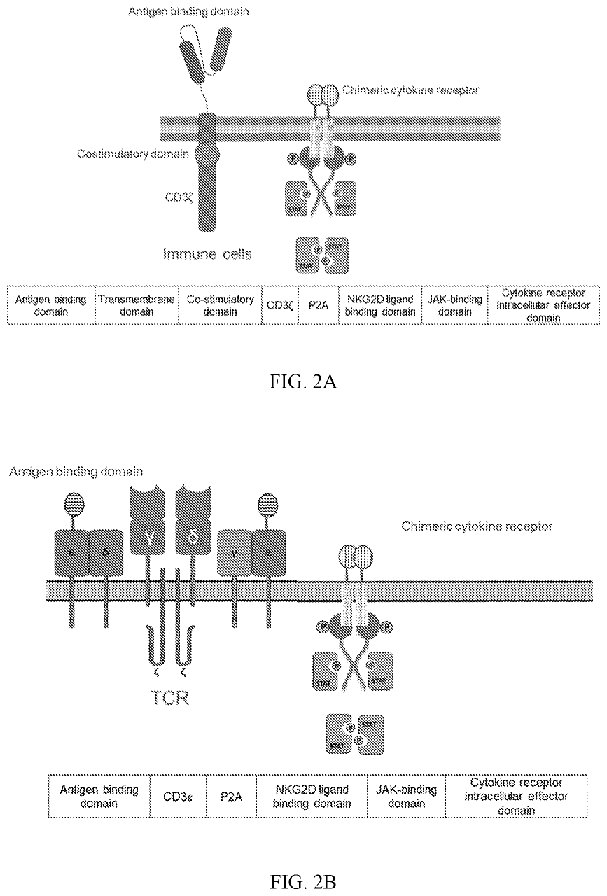

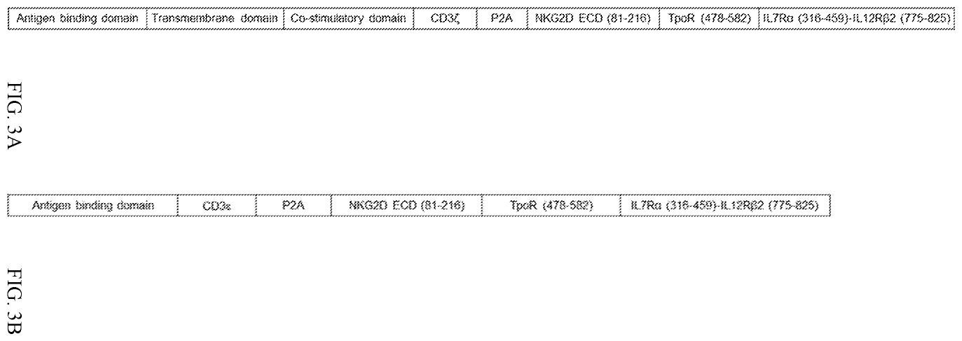

CART cells armored with different chimeric cytokine receptors were designed as shown in FIGS. 1A-1B, 2A-2B, and 3A-3B and SEQ ID NOs: 30-134, 136-140, 142, 144, 146, 148, 150 and 151. To generate viral particles comprising polynucleic acids encoding any of the systems disclosed herein lentivirus packaging plasmid mixture including pMDLg/pRRE (Addgene #11251), pRSV-Rev (Addgene #11253), and pMD2.G (Addgene #11259) were pre-mixed with a PLVX-EF1A (including target system) vector at a pre-optimized ratio with polyetherimide (PEI), and incubated at room temperature for 5 minutes. The transfection mix was added dropwise to 293-T cells and mixed gently. Transfected 293-T cells were incubated overnight at 37° C. and 5% CO₂. Twenty-four hours post-transfection, supernatants were collected and centrifuged at 4° C., 500 g for 10 min to remove any cellular debris. Centrifuged supernatants were filtered through a 0.45 m PES filter to concentrate the viral supernatants post ultracentrifugation. After centrifugation, the supernatants were carefully discarded and the virus pellets were rinsed with pre-chilled DPBS. The concentration of virus was measured. Virus was aliquoted and stored at −80° C. Viral titers were determined by functional transduction on a T cell line.

Briefly, the lentiviral vector was modified using pLVX-Puro (Clontech #632164) by replacing the original promoter with human elongation factor 1α promoter (hEF1α) and by removing the puromycin resistance gene with EcoRI and BamHI by GenScript. PLVX-EF1A was further subjected to the lentivirus packaging procedure as described above.

### 6.2. Example 2—T Cell Transduction and FACS Analysis of Transduced T Cells

γδ T cells or αβT cells were prepared by addition of 5 μM Zoledronate and 1000 IU/mL IL-2 to PBMCs and cultured for 14 days with periodical change of media supplemented with 1000 IU/mL IL-2. Alternatively, 76 T cells or αβT cells were isolated from PBMC or umbilical cord blood (UCB) and then stimulated by anti-γδ TCR antibody or anti-a TCR antibody and anti-CD3 (OKT3) followed by co-incubation of K562-based artificial antigen-presenting cells (aAPCs) at an 1:2 ratio for at least 10 days.

PBMCs were isolated by density centrifugation (lymphoprep) from leukapheresis material and cryopreserved. PBMCs were resuscitated and activated with zoledronic acid (5 μM) in cell culture media AIM-V supplemented with IL-2 (1000 IU/ml) and 5% human AB serum and kept in a humidified chamber (37° C., 5% CO₂). Forty-eight hours post-activation, cells were transduced with lentiviral vectors encoding the system of Example 1 at an MOI of 5 with 5 pg/ml polybrene. Such transduction procedure was repeated the next day followed by replenishment of fresh media containing IL-2 (1000 IU/ml) the second day after the transduction. Cells were cultured in AIM-V supplemented with IL-2 (1000 IU/ml) in a humidified chamber with periodical change of media as determined by the pH of the culture media for further expansion. Cells were harvested 10 days post-transduction and the total number, purity and transduction efficiency was determined. Cells were further enriched with a negative TCRγ/δ+ T cell or TCRαβ+ T cell isolation kit (Miltenyi Biotec) before future applications or cryopreserved.

### 6.3. Example 3—Quantification of Transgene Expression

On day 3 and onwards (typically day 3, 7 and 14) post transduction, cells were evaluated for expression of the system of Example 1 by flow cytometry. An aliquot of cells was collected from the culture before washed, pelleted, and resuspended in diluted antibodies (eBioscience) at a dilution factor of 100 in PBS+0.5% FBS−50-100 μl per sample. Resuspended cells were resuspended in about 50 to 100 μl of solution. Cell were incubated at 4° C. for 30 minutes. Viability dye eFluor780 or SYTOX Blue viability stain was also added according to manufacturer's instructions. Post-incubation, cells were washed twice in PBS and resuspended in 100 to 200 μl PBS for analysis. The mean fluorescence of the system was quantified by flow cytometry.

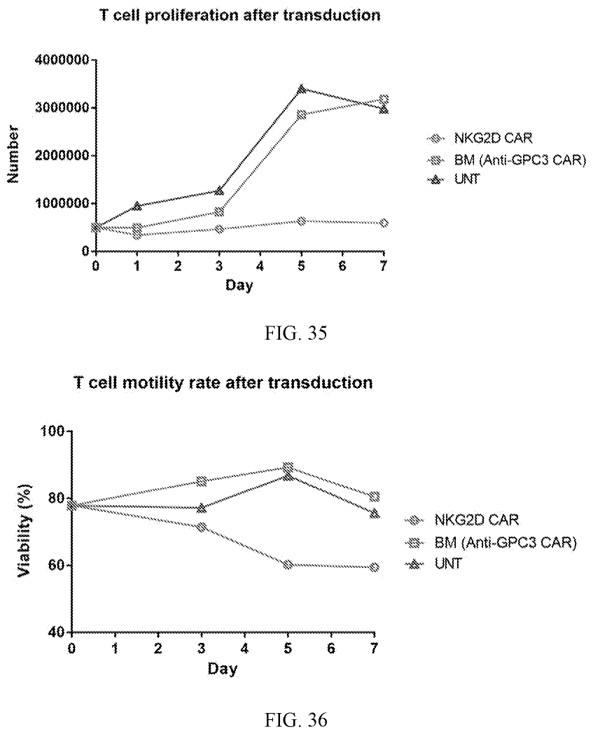

As shown in FIGS. 35 and 36, compared to anti-GPC3 CAR αβT cells, NKG2D CAR T cells have poor proliferation and viability during culture because of suicide or fratricide.

For anti-GPC3 CAR-T staining, cells were stained with PE anti-DYKDDDDK Tag Antibody (Biolegend). Flow cytometry analysis for all experiments was performed by using FlowJo (Tree Star, Inc.).

The positive rates of virus infected T cells expressing different CAR or with armor were shown in Table 1.

TABLE 1
Transfection efficiency of exemplary anti-GPC3 CAR T cells

| | Positive rate of T |
|---|---|
| Construct | cells transfection |
| BM CAR (γδT cells) | 50.3% |
| BM NKG2D-IL-7Rα--IL12Rβ2 (γδT cells) | 37.3% |

### 6.4. Example 4—Quantification of Ligands Expression on Tumor Cell Lines

Ligand surface expression of NKG2D on HCC cell lines huh7 was determined. Huh7 cells were stained with anti-MIC A/B antibody (Biolegend), anti-ULBP-1 antibody (R&D), anti-ULBP-3 antibody (R&D), anti-ULBP-2,5,6 antibody (R&D) or anti-CD155 antibody (Biolegend).

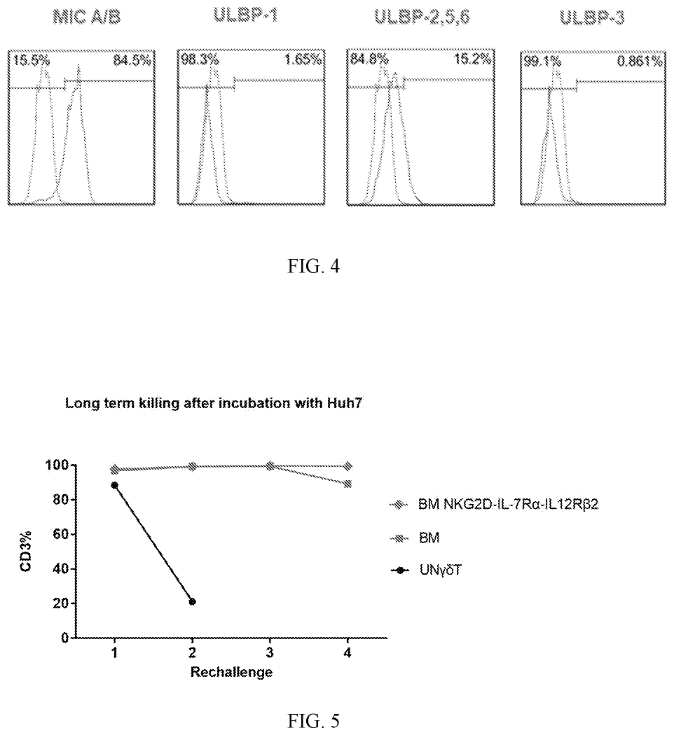

As shown in FIG. 4, huh7 cells moderately expressed NKG2D ligands, MIC A/B, ULBP-2, 5, 6.

### 6.5. Example 5—In Vitro Cytotoxicity Assay

For quick evaluation of anti-tumor activities of CAR-T cells (76 T cells or αβT cells) in vitro, FACS assay for cytotoxicity was performed. On day 8 after transduction, transduced T cells were harvested and co-incubated with target cells (GPC3⁺ HCC cell line: Huh7 and PLC-PRF-5, GPC3-HCC cell line: SK-HEP-1) at an E/T ratio (Effector: CAR-T/Target: Huh7, PLC-PRF-5 or SK-HEP-1) of 3:1, 1:1 or 0.3:1 for 16 hours. The target cells were pre-stained for CFSE. Un-transduced T cells (UNT) from the same batch were used as a negative control. The mixed cells were harvested and stained for 7-ADD. The cytotoxicity was calculated by the equation below: 7AAD+(E+T): the 7-ADD % in E/T co-incubation, 7AAD+(T): the 7-ADD % in target only. All anti-GPC3 CAR T cells (γδ T cells or αβT cells) show strong and comparable cytotoxicity against huh7 cells. Cytoxocity is calculated as: Cytotoxicity %=(7AAD+(E+T)−7AAD+(T))/(1−7AAD+(T))×100.

### 6.6. Example 6—IFN-γ, GM-CSF and TNF-α Secretion Detected by HTRF

In order to measure effector T-cell activation and proliferation, the production of effector cytokines such as IFN-γ, GM-CSF and TNF-α was measured. Supernatant from the in vitro cytotoxicity assay was collected to assess CAR-induced cytokine release. HTRF assays for IFN-7 (Cisbio, Cat #62HIFNGPEH), GM-CSF (Cisbio, Cat #62HGMCSFPEH) and TNFα (Cisbio, Cat #62HTNFAPEH) were performed according to the manufacturer's manual.

All anti-GPC3 CART cells (γδ T cells or αβT cells) exhibit potent killing activity against huh7 cells, and release IFN-γ, GM-CSF and TNF-α in response to huh7 cells. CAR T cells (γδ T cells or αβT cells) armored with NKG2D chimeric cytokine receptors showed similar level of TNF-α, IFN-γ and GM-CSF secretion as naked CAR T cells.

### 6.7. Example 7—Long-Term Cytotoxicity Assay

To evaluate the long-term killing efficacy of CAR T cells (γδ T cells or αβT cell), long-term co-culture assays were performed, which mimic the dynamic killing process in vivo. Transduced or non-transduced T cells (1×10⁵/well) were co-cultured with tumor cell lines (huh7, Raji or U937 cells, 1×10⁵/well) at an E:T ratio of 1:1 in 24-well plates, in the absence of exogenous cytokines (IL-2). Part of the cells were harvested and stained for CD3 after 2 or 3 days' co-culture. For serial co-culture assays, the remaining T cells were then re-challenged with fresh huh7 cells at the same E:T ratio. Co-cultures were carried on until tumor cells outgrew. The T cell proliferation rate at each time point was calculated by dividing the number of T cells at the time point by the number of T cells at the initial time point.

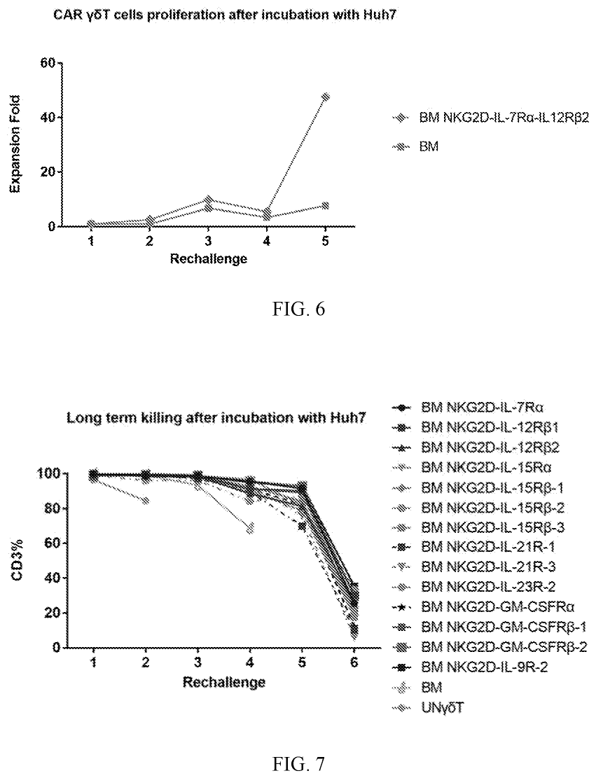

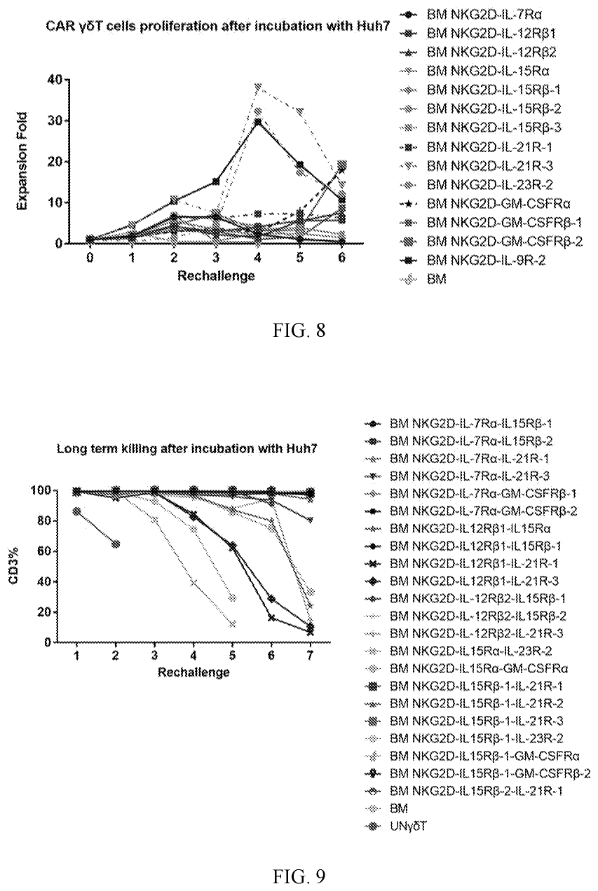

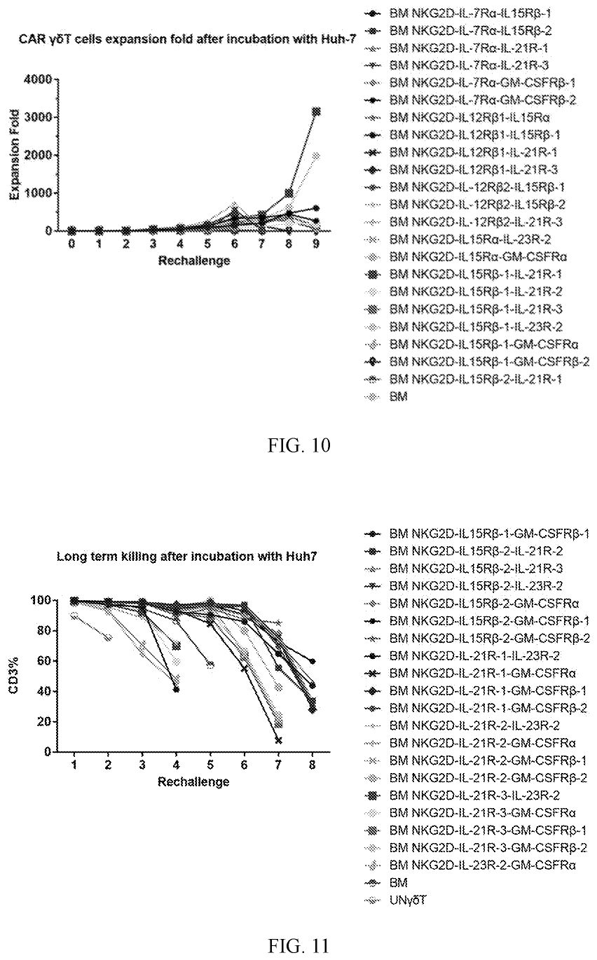

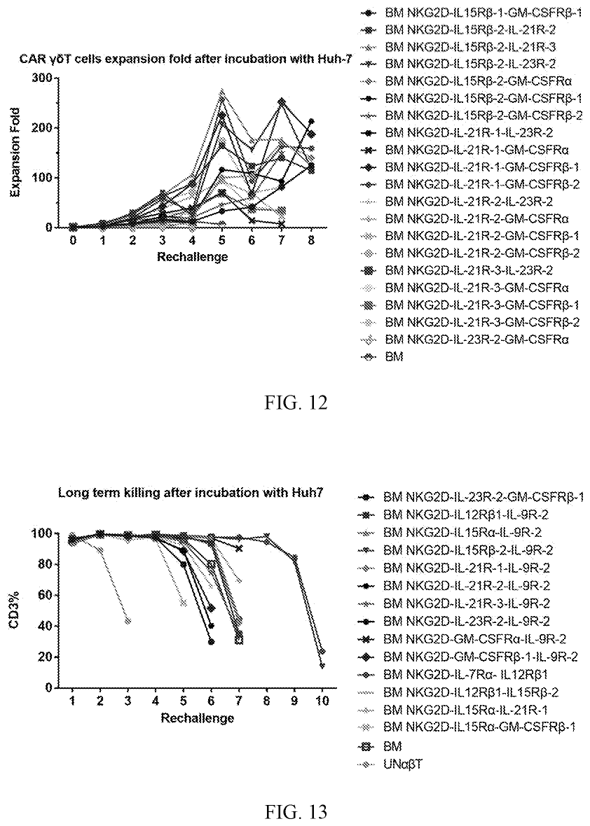

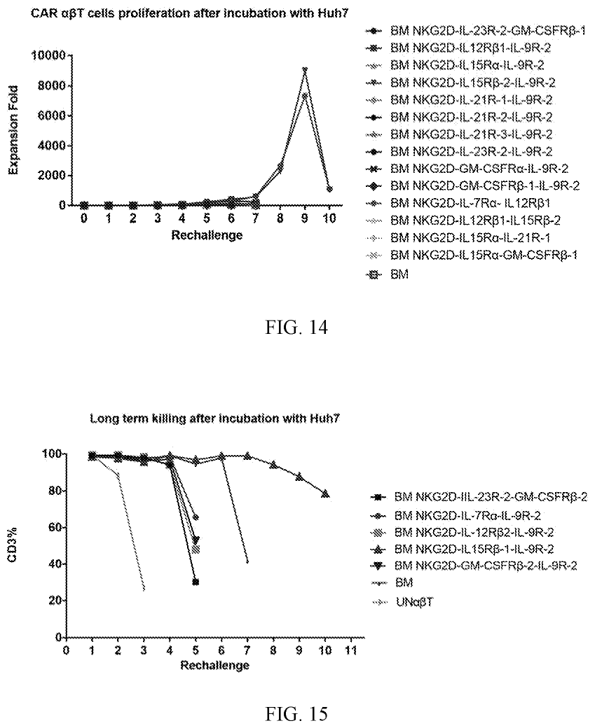

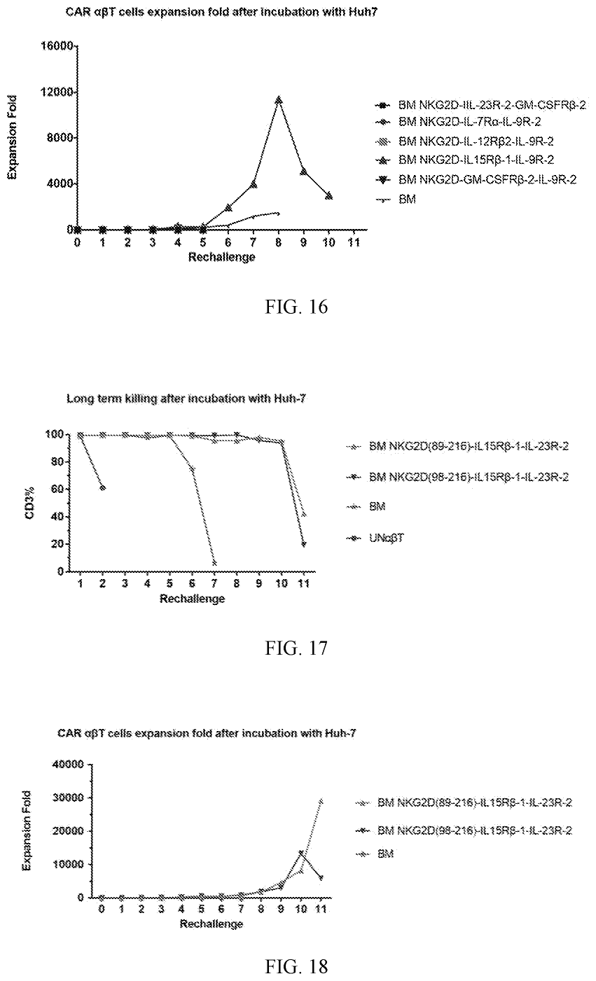

Long-term cytotoxicity of NKG2D chimeric single or two cytokine receptors armored BM CAR γδT cells or αβT cells by FACS detection are shown in FIGS. 5, 7, 9, 11, 13 and 15. Calculated T cells proliferation from the same experiment are shown in FIGS. 6, 8, 10, 12, 14 and 16. The data indicated that NKG2D chimeric cytokine receptors armors improved BM CAR T cells cytotoxicity and proliferation.

Long-term cytotoxicity of truncated NKG2D chimeric two cytokine receptors armored BM CAR αβT cells by FACS detection are shown in FIG. 17. Calculated T cells proliferation from the same experiment are shown in FIG. 18. The data indicated that truncated NKG2D chimeric cytokine receptors armors improved BM CAR T cells cytotoxicity and proliferation.

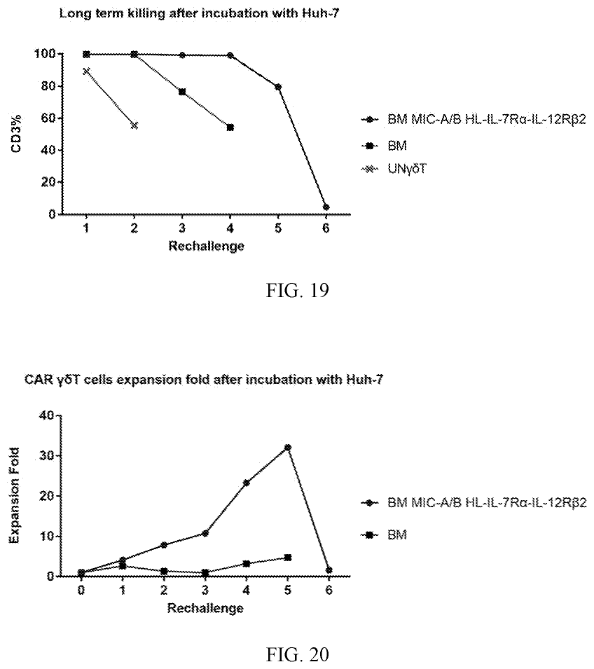

Long-term cytotoxicity of anti-MIC-A/B scFv chimeric two cytokine receptors armored BM CAR γδT cells by FACS detection are shown in FIG. 19. Calculated T cells proliferation from the same experiment are shown in FIG. 20. The data indicated that anti-MIC-A/B scFv chimeric cytokine receptors armors improved BM CAR T cells cytotoxicity and proliferation.

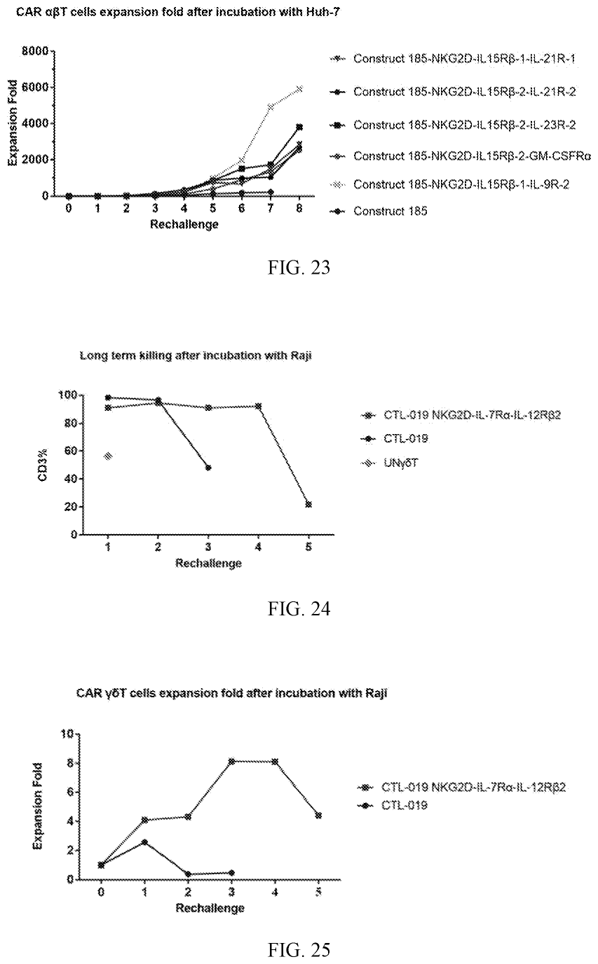

Long-term cytotoxicity of NKG2D chimeric two cytokine receptors armored construct 185 CAR αβT cells (an anti-GPC3 CAR αβT cells) by FACS detection are shown in FIG. 22. Calculated T cells proliferation from the same experiment are shown in FIG. 23. The data indicated that NKG2D chimeric two cytokine receptors armors improved construct 185 CAR CAR T cells cytotoxicity and proliferation.

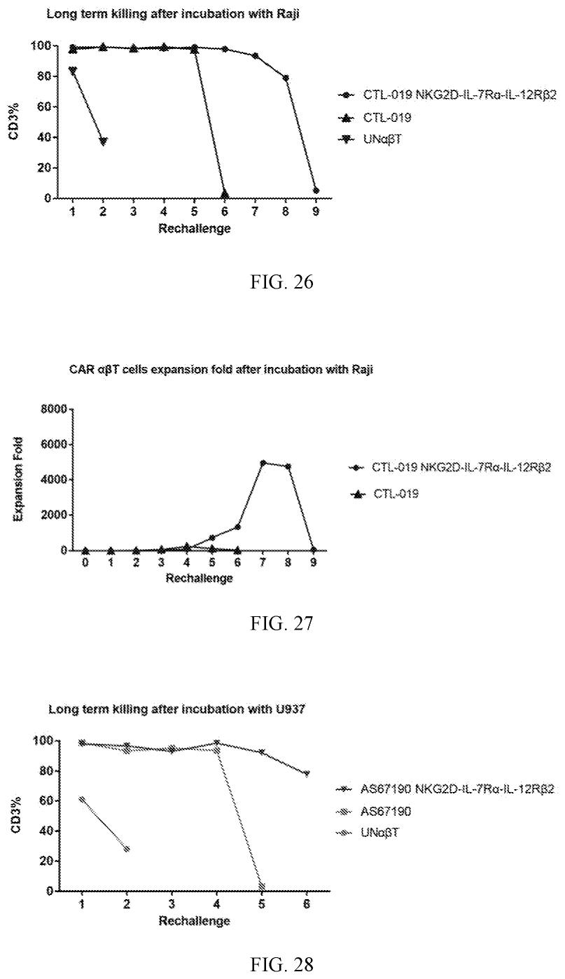

Long-term cytotoxicity of NKG2D chimeric two cytokine receptors armored CTL-019 CAR (an anti-CD19 CAR) 76T cells or αβT cells by FACS detection are shown in FIGS. 24 and 26. Calculated T cells proliferation from the same experiment are shown in FIGS. 25 and 27. The data indicated that NKG2D chimeric cytokine receptors armors improved CTL-019 CAR T cells cytotoxicity and proliferation.

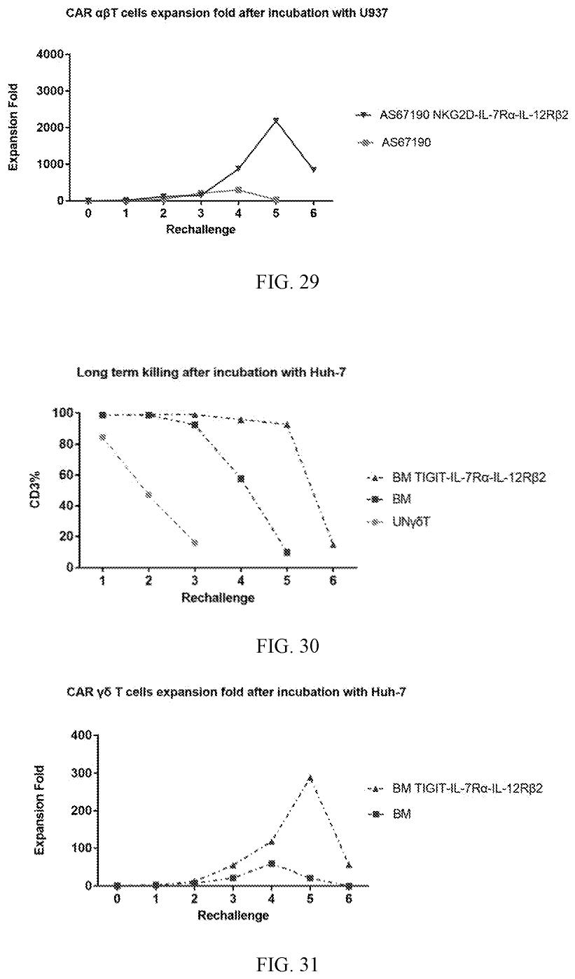

Long-term cytotoxicity of NKG2D chimeric two cytokine receptors armored AS67190 CAR αβT cells (an anti-CD33 CAR αβT cells) by FACS detection are shown in FIG. 28. Calculated T cells proliferation from the same experiment are shown in FIG. 29. The data indicated that NKG2D chimeric two cytokine receptors armors improved AS67190 CAR CAR T cells cytotoxicity and proliferation.

Long-term cytotoxicity of TIGIT chimeric two cytokine receptors armored BM CAR γδT cells by FACS detection are shown in FIG. 30. Calculated T cells proliferation from the same experiment are shown in FIG. 31. The data indicated that TIGIT chimeric cytokine receptors armors improved BM CAR T cells cytotoxicity and proliferation.

Long-term cytotoxicity of SIRP-α chimeric two cytokine receptors armored AS67190 CAR γδT cells by FACS detection are shown in FIG. 33. Calculated T cells proliferation from the same experiment are shown in FIG. 34. The data indicated that SIRP-α chimeric cytokine receptors armors improved AS67190 CAR T cells cytotoxicity and proliferation.

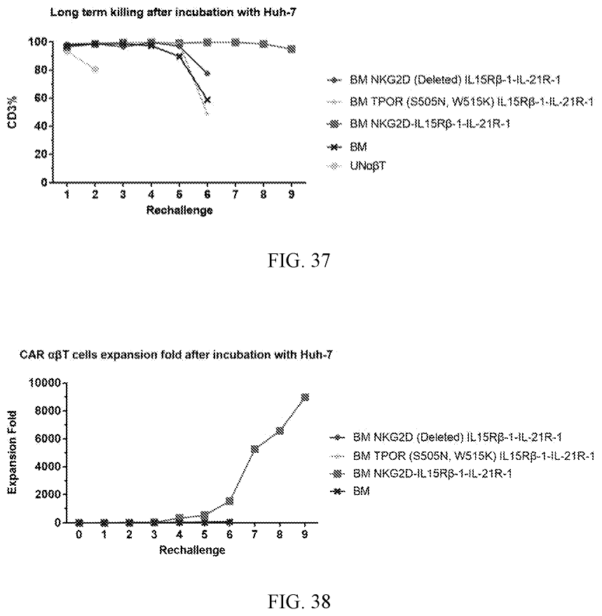

Long-term cytotoxicity of depleted NKG2D or constitutively active chimeric two cytokine receptors armored BM CAR αβT cells by FACS detection are shown in FIG. 37. Calculated T cells proliferation from the same experiment are shown in FIG. 38. The data indicated that NKG2D is significantly important to chimeric cytokine receptors armors.

### 6.8. Example 8—In Vivo Safety and Efficacy Evaluation

Anti-tumor activity of an exemplary anti-GPC3 CAR-T cells (γδ T cells or αβT cells) was assessed in vivo in a huh7 xenograft model. Briefly, 3 million (3×10⁶) huh7 cells were implanted subcutaneously on day 0 in NOD/SCID IL-2RγC null (NSG) mice. Ten days after tumor inoculation, mice were treated with intravenous injection of 1×10⁶ armored CAR-76 T or mock T cells or phosphate-buffered saline (PBS). Tumor dimensions were measured with calipers twice a week, and tumor volumes were calculated using the formula V=1/2(length×width2). Mice were euthanized when the mean tumor burden in the control mice reached 2,000 mm³. In addition, T cell proliferation was monitored via FACS analysis from plasma drawn from blood.

For toxicity evaluations, clinical symptoms were observed every day, while the animals' body weights. Blood (0.2 mL) were taken every week for detecting the humanized cytokine profiles (GM-CSF, IFN-γ and TNF-α) in mice.

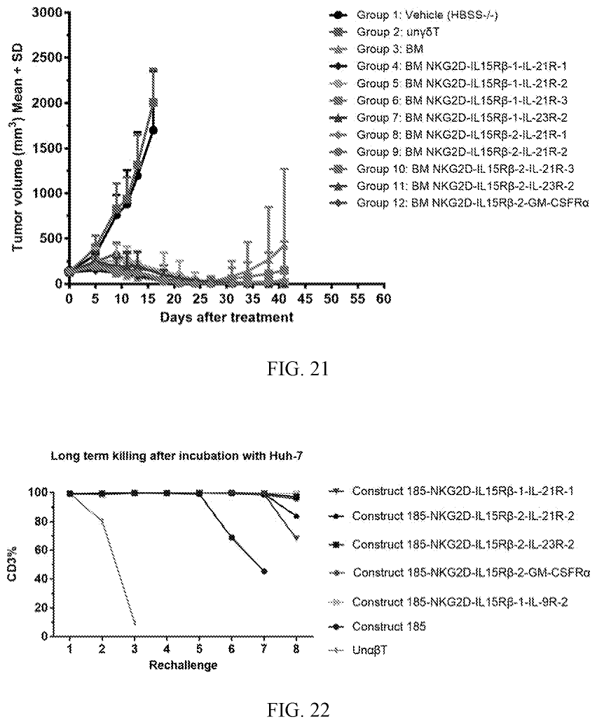

The results of anti-tumor effect of anti-GPC3 CAR γδT cells or anti-GPC3 CAR armored with NKG2D chimeric two cytokine receptors γδT cells in huh7 xenograft model were shown in FIG. 21. Unarmored CAR-76 T cells, alongside chimeric cytokine receptors armored CAR-γδ T cells inhibited tumor growth. Specifically, unarmored CAR-γδ T cells-treated mice reached tumor free but slowly repulsed, while NKG2D chimeric two cytokine receptors armored CAR-γδ T cells-treated mice reached tumor-free and remained healthy and tumor-free till the end of experimental observations.

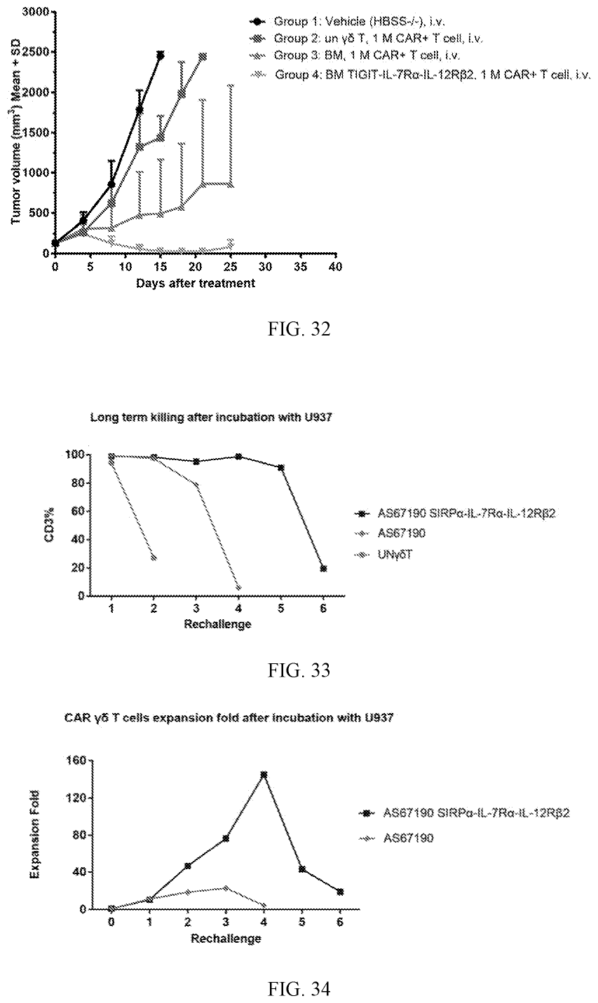

The result of anti-tumor effect of anti-GPC3 CAR γδT cells or anti-GPC3 CAR armored with TIGIT chimeriytokine receptors γδT cells in huh7 xenograft model was shown in FIG. 32. Unarmored CAR-γδ T cells, alongside chimeric cytokine receptors armored CAR-γδ T cells inhibited tumor growth. Specifically, unarmored CAR-γδ T cells-treated mice were slightly inhibited, while TIGIT chimeric two cytokine receptors armored CAR-γδ T cells-treated mice reached tumor-free.

In summary, chimeric cytokine receptors armored CAR-γδ T cells were demonstrated efficacious and safe in treating tumors as shown in the in vitro efficacy, in vivo efficacy, and safety tests.

From the foregoing, it will be appreciated that, although specific embodiments have been described herein for the purpose of illustration, various modifications may be made without deviating from the spirit and scope of what is provided herein. All of the references referred to above are incorporated herein by reference in their entireties.

SEQUENCE LISTING

SEQ ID NO: 1 (CD8α signal peptide amino acid sequence)
MALPVTALLLPLALLLHAARP
SEQ ID NO: 2 (GM-CSF signal peptide amino acid sequence)
MWLQSLLLLGTVACSIS
SEQ ID NO: 3 (IL-15 signal peptide amino acid sequence)
MRISKPHLRSISIQCYLCLLLNSHFLTEA
SEQ ID NO: 4 (FLAG tag amino acid sequence)
DYKDDDDK
SEQ ID NO: 5 (HA tag amino acid sequence)
YPYDVPDYA
SEQ ID NO: 6 (NKG2D 81-216 amino acid sequence)
SLFNQEVQIPLTESYCGPCPKNWICYKNNCYQFFDESKNWYESQASCMSQNASLLKVYS
KEDQDLLKLVKSYHWMGLVHIPTNGSWQWEDGSILSPNLLTIIEMQKGDCALYASSFKG
YIENCSTPNTYICMQRTV
SEQ ID NO: 7 (TpoR 478-582 amino acid sequence)
SDPTRVETATETAWISLVTALHLVLGLSAVLGLLLLRWQFPAHYRRLRHALWPSLPDLHRV
LGQYLRDTAALSPPKATVSDTCEEVEPSLLEILPKSSERTPLPL
SEQ ID NO: 8 (IL7Ra 316-459 amino acid sequence)
ARDEVEGFLQDTFPQQLEESEKQRLGGDVQSPNCPSEDVVITPESFGRDSSLTCLAGNVS
ACDAPILSSSRSLDCRESGKNGPHVYQDLLLSLGTTNSTLPPPFSLQSGILTLNPVAQGQPI
LTSLGSNQEEAYVTMSSFYQNQ
SEQ ID NO: 9 (IL9R-1 310-464 amino acid sequence)
MFFQPLYSVHNGNFQTWMGAHGAGVLLSQDCAGTPQGALEPCVQEATALLTCGPARPW
KSVALEEEQEGPGTRLPGNLSSEDVLPAGCTEWRVQTLAYLPQEDWAPTSLTRPAPPDSE
GSRSSSSSSSSNNNNYCALGCYGGWHLSALPGNTQSS
SEQ ID NO: 10 (IL9R-2 391-464 amino acid sequence)
DVLPAGCTEWRVQTLAYLPQEDWAPTSLTRPAPPDSEGSRSSSSSSSSNNNNYCALGCYG
GWHLSALPGNTQSS
SEQ ID NO: 11 (IL9R-3 310-337, 391-464 amino acid sequence)
MFFQPLYSVHNGNFQTWMGAHGAGVLLSDVLPAGCTEWRVQTLAYLPQEDWAPTSLTR
PAPPDSEGSRSSSSSSSSNNNNYCALGCYGGWHLSALPGNTQSS
SEQ ID NO: 12 (IL12Rβ1 612-662 amino acid sequence)
SLQEALVVEMSWDKGERTEPLEKTELPEGAPELALDTELSLEDGDRCKAKM
SEQ ID NO: 13 (IL12Rβ2 775-825 amino acid sequence)
SDPKPENPACPWTVLPAGDLPTHDGYLPSNIDDLPSHEAPLADSLEELEPQ
SEQ ID NO: 14 (IL15Rα 229-267 amino acid sequence)
KSRQTPPLASVEMEAMEALPVTWGTSSRDEDLENCSHHL
SEQ ID NO: 15 (IL15Rβ-1 333-551 amino acid sequence)
VTQLLLQQDKVPEPASLSSNHSLTSCFTNQGYFFFHLPDALEIEACQVYFTYDPYSEEDPD
EGVAGAPTGSSPQPLQPLSGEDDAYCTFPSRDDLLLFSPSLLGGPSPPSTAPGGSGAGEER
MPPSLQERVPRDWDPQPLGPPTPGVPDLVDFQPPPELVLREAGEEVPDAGPREGVSFPWS
RPPGQGEFRALNARLPLNTDAYLSLQELQGQDPTHLV
SEQ ID NO: 16 (IL15Rβ-2 345-431, 521-551 amino acid sequence)
EPASLSSNHSLTSCFTNQGYFFFHLPDALEIEACQVYFTYDPYSEEDPDEGVAGAPTGSSP
QPLQPLSGEDDAYCTFPSRDDLLLFSEFRALNARLPLNTDAYLSLQELQGQDPTHLV
SEQ ID NO: 17 (IL15Rβ-3 401-431, 521-551 amino acid sequence)
TGSSPQPLQPLSGEDDAYCTFPSRDDLLLFSEFRALNARLPLNTDAYLSLQELQGQDPTHL
V
SEQ ID NO: 18 (IL-18Rα 361-530 amino acid sequence)
HLTRRDETLTDGKTYDAFVSYLKECRPENGEEHTFAVEILPRVLEKHFGYKLCIFERDVVP
GGAVVDEIHSLIEKSRRLIIVLSKSYMSNEVRYELESGLHEALVERKIKIILIEFTPVTDFTFL
PQSLKLLKSHRVLKWKADKSLSYNSRFWKNLLYLMPAKTVKPGRD
SEQ ID NO: 19 (IL-18Rβ 391-570 amino acid sequence)
LYRHWIEIVLLYRTYQSKDQTLGDKKDFDAFVSYAKWSSFPSEATSSLSEEHLALSLFPDV
LENKYGYSLCLLERDVAPGGVYAEDIVSIIKRSRRGIFILSPNYVNGPSIFELQAAVNLALD
DQTLKLILIKFCYFQEPESLPHLVKKALRVLPTVTWRGLKSVPPNSRFWAKMRYHMPVK
NSQGFTWN
SEQ ID NO: 20 (IL-21R-1 301-411 amino acid sequence)
LELGPWSPEVPSTLEVYSCHPPRSPAKRLQLTELQEPAELVESDGVPKPSFWPTAQNSGG
SAYSEERDRPYGLVSIDTVTVLDAEGPCTWPCSCEDDGYPALDLDAGLEPS
SEQ ID NO: 21 (IL-21R-2 504-538 amino acid sequence)
ECDFTSPGDEGPPRSYLRQWVVIPPPLSSPGPQAS
SEQ ID NO: 22 (IL-21R-3 301-411, 504-538 amino acid sequence)
LELGPWSPEVPSTLEVYSCHPPRSPAKRLQLTELQEPAELVESDGVPKPSFWPTAQNSGG
SAYSEERDRPYGLVSIDTVTVLDAEGPCTWPCSCEDDGYPALDLDAGLEPSECDFTSPGD
EGPPRSYLRQWVVIPPPLSSPGPQAS
SEQ ID NO: 23 (IL-23R-1 381-500 amino acid sequence)
RTGIKRRILLLIPKWLYEDIPNMKNSNVVKMLQENSELMNNNSSEQVLYVDPMITEIKEIF
IPEHKPTDYKKENTGPLETRDYPQNSLFDNTTVVYIPDLNTGYKPQISNFLPEGSHLSN
SEQ ID NO: 24 (IL-23R-2 593-625 amino acid sequence)
EFVSCLGIVNEELPSINTYFPQNILESHENRIS
SEQ ID NO: 25 (IL-23R-3 381-500, 593-625 amino acid sequence)
RTGIKRRILLLIPKWLYEDIPNMKNSNVVKMLQENSELMNNNSSEQVLYVDPMITEIKEIF
IPEHKPTDYKKENTGPLETRDYPQNSLFDNTTVVYIPDLNTGYKPQISNFLPEGSHLSNEF
VSCLGIVNEELPSINTYFPQNILESHENRIS
SEQ ID NO: 26 (GM-CSFRα 371-400 amino acid sequence)
EVEDEIIWEEFTPEEGKGYREEVLTVKEIT
SEQ ID NO: 27 (GM-CSFRβ-1 701-729, 751-779, 809-838, 867-897 amino acid sequence)
DTEDPGVASGYVSSADLVFTPNSGASSVSGPPGAPGPVKSGFEGYVELPPIEGRSPRSQPE
GLLVLQQVGDYCFLPGLGPGPLSLRSKQVPVIQLFKALKQQDYLSLPPWEVNKPGEVC
SEQ ID NO: 28 (GM-CSFRβ-2 581-644, 701-729, 751-779, 809-838, 867-897 amino acid
sequence)
EKQASSFDFNGPYLGPPHSRSLPDILGQPEPPQEGGSQKSPPPGSLEYLCLPAGGQVQLVP
LAQDTEDPGVASGYVSSADLVFTPNSGASSVSGPPGAPGPVKSGFEGYVELPPIEGRSPRS
QPEGLLVLQQVGDYCFLPGLGPGPLSLRSKQVPVIQLFKALKQQDYLSLPPWEVNKPGE
VC
SEQ ID NO: 29 (BM CAR amino acid sequence)
MALPVTALLLPLALLLHAARPDVVMTQSPLSLPVTPGEPASISCRSSQSL VHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSS
LRSEDTAVYYCTRFYSYTYWGQGTLVTVSSTTTPAPRPPTPAPTIASQPLSLRPEACRPAA
GGAVHTRGLDFACDIYIWAPLAGTCGVLLLSLVITLYCKRGRKKLLYIFKQPFMRPVQTT
QEEDGCSCRFPEEEEGGCELRVKFSRSADAPAYKQGQNQLYNELNLGRREEYDVLDKR
RGRDPEMGGKPRRKNPQEGLYNELQKDKMAEAYSEIGMKGERRRGKGHDGLYQGLST
ATKDTYDALHMQALPPR
SEQ ID NO: 30 (BM NKG2D-IL7Rα-IL12Rβ2 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLARD
EVEGFLQDTFPQQLEESEKQRLGGDVQSPNCPSEDVVITPESFGRDSSLTCLAGNVSACD
APILSSSRSLDCRESGKNGPHVYQDLLLSLGTTNSTLPPPFSLQSGILTLNPVAQGQPILTSL
GSNQEEAYVTMSSFYQNQSDPKPENPACPWTVLPAGDLPTHDGYLPSNIDDLPSHEAPLA
DSLEELEPQ
SEQ ID NO: 31 (BM NKG2D-IL-7Rα amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLARD
EVEGFLQDTFPQQLEESEKQRLGGDVQSPNCPSEDVVITPESFGRDSSLTCLAGNVSACD
APILSSSRSLDCRESGKNGPHVYQDLLLSLGTTNSTLPPPFSLQSGILTLNPVAQGQPILTSL
GSNQEEAYVTMSSFYQNQ
SEQ ID NO: 32 (BM NKG2D-IL12Rβ1 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLSLQE
ALVVEMSWDKGERTEPLEKTELPEGAPELALDTELSLEDGDRCKAKM
SEQ ID NO: 33 (BM NKG2D-IL-12Rβ2 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLSDPK
PENPACPWTVLPAGDLPTHDGYLPSNIDDLPSHEAPLADSLEELEPQ
SEQ ID NO: 34 (BM NKG2D-IL15Rα amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLKSR
QTPPLASVEMEAMEALPVTWGTSSRDEDLENCSHHL
SEQ ID NO: 35 (BM NKG2D-IL15Rβ-1 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLVTQL
LLQQDKVPEPASLSSNHSLTSCFTNQGYFFFHLPDALEIEACQVYFTYDPYSEEDPDEGVA
GAPTGSSPQPLQPLSGEDDAYCTFPSRDDLLLFSPSLLGGPSPPSTAPGGSGAGEERMPPSL
QERVPRDWDPQPLGPPTPGVPDLVDFQPPPELVLREAGEEVPDAGPREGVSFPWSRPPGQ
GEFRALNARLPLNTDAYLSLQELQGQDPTHLV
SEQ ID NO: 36 (BM NKG2D-IL15Rβ-2 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLEPAS
LSSNHSLTSCFTNQGYFFFHLPDALEIEACQVYFTYDPYSEEDPDEGVAGAPTGSSPQPLQ
PLSGEDDAYCTFPSRDDLLLFSEFRALNARLPLNTDAYLSLQELQGQDPTHLV
SEQ ID NO: 37 (BM NKG2D-IL15Rβ-3 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLTGSS
PQPLQPLSGEDDAYCTFPSRDDLLLFSEFRALNARLPLNTDAYLSLQELQGQDPTHLV
SEQ ID NO: 38 (BM NKG2D-IL-18Rα amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLHLTR
RDETLTDGKTYDAFVSYLKECRPENGEEHTFAVEILPRVLEKHFGYKLCIFERDVVPGGA
VVDEIHSLIEKSRRLIIVLSKSYMSNEVRYELESGLHEALVERKIKIILIEFTPVTDFTFLPQS
LKLLKSHRVLKWKADKSLSYNSRFWKNLLYLMPAKTVKPGRD
SEQ ID NO: 39 (BM NKG2D-IL-18Rβ amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLLYR
HWIEIVLLYRTYQSKDQTLGDKKDFDAFVSYAKWSSFPSEATSSLSEEHLALSLFPDVLEN
KYGYSLCLLERDVAPGGVYAEDIVSIIKRSRRGIFILSPNYVNGPSIFELQAAVNLALDDQT
LKLILIKFCYFQEPESLPHLVKKALRVLPTVTWRGLKSVPPNSRFWAKMRYHMPVKNSQ
GFTWN
SEQ ID NO: 40 (BM NKG2D-IL-21R-1 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLLELG
PWSPEVPSTLEVYSCHPPRSPAKRLQLTELQEPAELVESDGVPKPSFWPTAQNSGGSAYSE
ERDRPYGLVSIDTVTVLDAEGPCTWPCSCEDDGYPALDLDAGLEPS
SEQ ID NO: 41 (BM NKG2D-IL-21R-2 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLECDF
TSPGDEGPPRSYLRQWVVIPPPLSSPGPQAS
SEQ ID NO: 42 (BM NKG2D-IL-21R-3 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLLELG
PWSPEVPSTLEVYSCHPPRSPAKRLQLTELQEPAELVESDGVPKPSFWPTAQNSGGSAYSE
ERDRPYGLVSIDTVTVLDAEGPCTWPCSCEDDGYPALDLDAGLEPSECDFTSPGDEGPPR
SYLRQWVVIPPPLSSPGPQAS
SEQ ID NO: 43 (BM NKG2D-IL-23R-1 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLRTGI
KRRILLLIPKWLYEDIPNMKNSNVVKMLQENSELMNNNSSEQVLYVDPMITEIKEIFIPEH
KPTDYKKENTGPLETRDYPQNSLFDNTTVVYIPDLNTGYKPQISNFLPEGSHLSN
SEQ ID NO: 44 (BM NKG2D-IL-23R-2 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLEFVS
CLGIVNEELPSINTYFPQNILESHENRIS
SEQ ID NO: 45 (BM NKG2D-IL-23R-3 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLRTGI
KRRILLLIPKWLYEDIPNMKNSNVVKMLQENSELMNNNSSEQVLYVDPMITEIKEIFIPEH
KPTDYKKENTGPLETRDYPQNSLFDNTTVVYIPDLNTGYKPQISNFLPEGSHLSNEFVSCL
GIVNEELPSINTYFPQNILESHENRIS
SEQ ID NO: 46 (BM NKG2D-GM-CSFRα amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLEVE
DEIIWEEFTPEEGKGYREEVLTVKEIT
SEQ ID NO: 47 (BM NKG2D-GM-CSFRβ-1 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLDTE
DPGVASGYVSSADLVFTPNSGASSVSGPPGAPGPVKSGFEGYVELPPIEGRSPRSQPEGLL
VLQQVGDYCFLPGLGPGPLSLRSKQVPVIQLFKALKQQDYLSLPPWEVNKPGEVC
SEQ ID NO: 48 (BM NKG2D-GM-CSFRβ-2 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLEKQ
ASSFDFNGPYLGPPHSRSLPDILGQPEPPQEGGSQKSPPPGSLEYLCLPAGGQVQLVPLAQ
DTEDPGVASGYVSSADLVFTPNSGASSVSGPPGAPGPVKSGFEGYVELPPIEGRSPRSQPE
GLLVLQQVGDYCFLPGLGPGPLSLRSKQVPVIQLFKALKQQDYLSLPPWEVNKPGEVC
SEQ ID NO: 49 (BM NKG2D-IL-9R-1 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLMFF
QPLYSVHNGNFQTWMGAHGAGVLLSQDCAGTPQGALEPCVQEATALLTCGPARPWKSV
ALEEEQEGPGTRLPGNLSSEDVLPAGCTEWRVQTLAYLPQEDWAPTSLTRPAPPDSEGSR
SSSSSSSSNNNNYCALGCYGGWHLSALPGNTQSS
SEQ ID NO: 50 (BM NKG2D-IL-9R-2 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLDVLP
AGCTEWRVQTLAYLPQEDWAPTSLTRPAPPDSEGSRSSSSSSSSNNNNYCALGCYGGWH
LSALPGNTQSS
SEQ ID NO: 51 (BM NKG2D-IL-9R-3 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLMFF
QPLYSVHNGNFQTWMGAHGAGVLLSDVLPAGCTEWRVQTLAYLPQEDWAPTSLTRPAP
PDSEGSRSSSSSSSSNNNNYCALGCYGGWHLSALPGNTQSS
SEQ ID NO: 52 (BM NKG2D-IL-7Rα-IL12Rβ1 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLARD
EVEGFLQDTFPQQLEESEKQRLGGDVQSPNCPSEDVVITPESFGRDSSLTCLAGNVSACD
APILSSSRSLDCRESGKNGPHVYQDLLLSLGTTNSTLPPPFSLQSGILTLNPVAQGQPILTSL
GSNQEEAYVTMSSFYQNQSLQEALVVEMSWDKGERTEPLEKTELPEGAPELALDTELSL
EDGDRCKAKM
SEQ ID NO: 53 (BM NKG2D-IL-7Rα-IL15Rα amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLARD
EVEGFLQDTFPQQLEESEKQRLGGDVQSPNCPSEDVVITPESFGRDSSLTCLAGNVSACD
APILSSSRSLDCRESGKNGPHVYQDLLLSLGTTNSTLPPPFSLQSGILTLNPVAQGQPILTSL
GSNQEEAYVTMSSFYQNQKSRQTPPLASVEMEAMEALPVTWGTSSRDEDLENCSHHL
SEQ ID NO: 54 (BM NKG2D-IL-7Rα-IL15R7Rα-1 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLARD
EVEGFLQDTFPQQLEESEKQRLGGDVQSPNCPSEDVVITPESFGRDSSLTCLAGNVSACD
APILSSSRSLDCRESGKNGPHVYQDLLLSLGTTNSTLPPPFSLQSGILTLNPVAQGQPILTSL
GSNQEEAYVTMSSFYQNQVTQLLLQQDKVPEPASLSSNHSLTSCFTNQGYFFFHLPDALE
IEACQVYFTYDPYSEEDPDEGVAGAPTGSSPQPLQPLSGEDDAYCTFPSRDDLLLFSPSLL
GGPSPPSTAPGGSGAGEERMPPSLQERVPRDWDPQPLGPPTPGVPDLVDFQPPPELVLREA
GEEVPDAGPREGVSFPWSRPPGQGEFRALNARLPLNTDAYLSLQELQGQDPTHLV
SEQ ID NO: 55 (BM NKG2D-IL-7Rα-IL15R7Rα-2 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLARD
EVEGFLQDTFPQQLEESEKQRLGGDVQSPNCPSEDVVITPESFGRDSSLTCLAGNVSACD
APILSSSRSLDCRESGKNGPHVYQDLLLSLGTTNSTLPPPFSLQSGILTLNPVAQGQPILTSL
GSNQEEAYVTMSSFYQNQEPASLSSNHSLTSCFTNQGYFFFHLPDALEIEACQVYFTYDP
YSEEDPDEGVAGAPTGSSPQPLQPLSGEDDAYCTFPSRDDLLLFSEFRALNARLPLNTDAY
LSLQELQGQDPTHLV
SEQ ID NO: 56 (BM NKG2D-IL-7Rα-IL-21R-1 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLARD
EVEGFLQDTFPQQLEESEKQRLGGDVQSPNCPSEDVVITPESFGRDSSLTCLAGNVSACD
APILSSSRSLDCRESGKNGPHVYQDLLLSLGTTNSTLPPPFSLQSGILTLNPVAQGQPILTSL
GSNQEEAYVTMSSFYQNQLELGPWSPEVPSTLEVYSCHPPRSPAKRLQLTELQEPAELVES
DGVPKPSFWPTAQNSGGSAYSEERDRPYGLVSIDTVTVLDAEGPCTWPCSCEDDGYPAL
DLDAGLEPS
SEQ ID NO: 57 (BM NKG2D-IL-7Rα-IL-21R-2 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLARD
EVEGFLQDTFPQQLEESEKQRLGGDVQSPNCPSEDVVITPESFGRDSSLTCLAGNVSACD
APILSSSRSLDCRESGKNGPHVYQDLLLSLGTTNSTLPPPFSLQSGILTLNPVAQGQPILTSL
GSNQEEAYVTMSSFYQNQECDFTSPGDEGPPRSYLRQWVVIPPPLSSPGPQAS
SEQ ID NO: 58 (BM NKG2D-IL-7Rα-IL-21R-3 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLARD
EVEGFLQDTFPQQLEESEKQRLGGDVQSPNCPSEDVVITPESFGRDSSLTCLAGNVSACD
APILSSSRSLDCRESGKNGPHVYQDLLLSLGTTNSTLPPPFSLQSGILTLNPVAQGQPILTSL
GSNQEEAYVTMSSFYQNQLELGPWSPEVPSTLEVYSCHPPRSPAKRLQLTELQEPAELVES
DGVPKPSFWPTAQNSGGSAYSEERDRPYGLVSIDTVTVLDAEGPCTWPCSCEDDGYPAL
DLDAGLEPSECDFTSPGDEGPPRSYLRQWVVIPPPLSSPGPQAS
SEQ ID NO: 59 (BM NKG2D-IL-7Rα-IL-23R-2 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLARD
EVEGFLQDTFPQQLEESEKQRLGGDVQSPNCPSEDVVITPESFGRDSSLTCLAGNVSACD
APILSSSRSLDCRESGKNGPHVYQDLLLSLGTTNSTLPPPFSLQSGILTLNPVAQGQPILTSL
GSNQEEAYVTMSSFYQNQEFVSCLGIVNEELPSINTYFPQNILESHENRIS
SEQ ID NO: 60 (BM NKG2D-IL-7Rα-GM-CSFRα amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLARD
EVEGFLQDTFPQQLEESEKQRLGGDVQSPNCPSEDVVITPESFGRDSSLTCLAGNVSACD
APILSSSRSLDCRESGKNGPHVYQDLLLSLGTTNSTLPPPFSLQSGILTLNPVAQGQPILTSL
GSNQEEAYVTMSSFYQNQEVEDEIIWEEFTPEEGKGYREEVLTVKEIT
SEQ ID NO: 61 (BM NKG2D-IL-7Rα-GM-CSFRβ-1 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLARD
EVEGFLQDTFPQQLEESEKQRLGGDVQSPNCPSEDVVITPESFGRDSSLTCLAGNVSACD
APILSSSRSLDCRESGKNGPHVYQDLLLSLGTTNSTLPPPFSLQSGILTLNPVAQGQPILTSL
GSNQEEAYVTMSSFYQNQDTEDPGVASGYVSSADLVFTPNSGASSVSGPPGAPGPVKSGF
EGYVELPPIEGRSPRSQPEGLLVLQQVGDYCFLPGLGPGPLSLRSKQVPVIQLFKALKQQD
YLSLPPWEVNKPGEVC
SEQ ID NO: 62 (BM NKG2D-IL-7Rα-GM-CSFRβ-2 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLARD
EVEGFLQDTFPQQLEESEKQRLGGDVQSPNCPSEDVVITPESFGRDSSLTCLAGNVSACD
APILSSSRSLDCRESGKNGPHVYQDLLLSLGTTNSTLPPPFSLQSGILTLNPVAQGQPILTSL
GSNQEEAYVTMSSFYQNQEKQASSFDFNGPYLGPPHSRSLPDILGQPEPPQEGGSQKSPPP
GSLEYLCLPAGGQVQLVPLAQDTEDPGVASGYVSSADLVFTPNSGASSVSGPPGAPGPVK
SGFEGYVELPPIEGRSPRSQPEGLLVLQQVGDYCFLPGLGPGPLSLRSKQVPVIQLFKALK
QQDYLSLPPWEVNKPGEVC
SEQ ID NO: 63 (BM NKG2D-IL12Rβ1-IL15Rα amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLSLQE
ALVVEMSWDKGERTEPLEKTELPEGAPELALDTELSLEDGDRCKAKMKSRQTPPLASVE
MEAMEALPVTWGTSSRDEDLENCSHHL
SEQ ID NO: 64 (BM NKG2D-IL12Rβ1-IL15Rβ-1 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLSLQE
ALVVEMSWDKGERTEPLEKTELPEGAPELALDTELSLEDGDRCKAKMVTQLLLQQDKV
PEPASLSSNHSLTSCFTNQGYFFFHLPDALEIEACQVYFTYDPYSEEDPDEGVAGAPTGSSP
QPLQPLSGEDDAYCTFPSRDDLLLFSPSLLGGPSPPSTAPGGSGAGEERMPPSLQERVPRD
WDPQPLGPPTPGVPDLVDFQPPPELVLREAGEEVPDAGPREGVSFPWSRPPGQGEFRALN
ARLPLNTDAYLSLQELQGQDPTHLV
SEQ ID NO: 65 (BM NKG2D-IL12Rβ1-IL15Rβ-2 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLSLQE
ALVVEMSWDKGERTEPLEKTELPEGAPELALDTELSLEDGDRCKAKMEPASLSSNHSLTS
CFTNQGYFFFHLPDALEIEACQVYFTYDPYSEEDPDEGVAGAPTGSSPQPLQPLSGEDDA
YCTFPSRDDLLLFSEFRALNARLPLNTDAYLSLQELQGQDPTHLV
SEQ ID NO: 66 (BM NKG2D-IL12Rβ1-IL-21R-1 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLSLQE
ALVVEMSWDKGERTEPLEKTELPEGAPELALDTELSLEDGDRCKAKMLELGPWSPEVPS
TLEVYSCHPPRSPAKRLQLTELQEPAELVESDGVPKPSFWPTAQNSGGSAYSEERDRPYGL
VSIDTVTVLDAEGPCTWPCSCEDDGYPALDLDAGLEPS
SEQ ID NO: 67 (BM NKG2D-IL12Rβ1-IL-21R-2 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLSLQE
ALVVEMSWDKGERTEPLEKTELPEGAPELALDTELSLEDGDRCKAKMECDFTSPGDEGP
PRSYLRQWVVIPPPLSSPGPQAS
SEQ ID NO: 68 (BM NKG2D-IL12Rβ1-IL-21R-3 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLSLQE
ALVVEMSWDKGERTEPLEKTELPEGAPELALDTELSLEDGDRCKAKMLELGPWSPEVPS
TLEVYSCHPPRSPAKRLQLTELQEPAELVESDGVPKPSFWPTAQNSGGSAYSEERDRPYGL
VSIDTVTVLDAEGPCTWPCSCEDDGYPALDLDAGLEPSECDFTSPGDEGPPRSYLRQWV
VIPPPLSSPGPQAS
SEQ ID NO: 69 (BM NKG2D-IL12Rβ1-IL-23R-2 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLSLQE
ALVVEMSWDKGERTEPLEKTELPEGAPELALDTELSLEDGDRCKAKMEFVSCLGIVNEE
LPSINTYFPQNILESHENRIS
SEQ ID NO: 70 (BM NKG2D-IL12Rβ1-GM-CSFRα amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLSLQE
ALVVEMSWDKGERTEPLEKTELPEGAPELALDTELSLEDGDRCKAKMEVEDEIIWEEFTP
EEGKGYREEVLTVKEIT
SEQ ID NO: 71 (BM NKG2D-IL12Rβ1-GM-CSFRβ-1 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLSLQE
ALVVEMSWDKGERTEPLEKTELPEGAPELALDTELSLEDGDRCKAKMDTEDPGVASGY
VSSADLVFTPNSGASSVSGPPGAPGPVKSGFEGYVELPPIEGRSPRSQPEGLLVLQQVGDY
CFLPGLGPGPLSLRSKQVPVIQLFKALKQQDYLSLPPWEVNKPGEVC
SEQ ID NO: 72 (BM NKG2D-IL12Rβ1-GM-CSFRβ-2 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLSLQE
ALVVEMSWDKGERTEPLEKTELPEGAPELALDTELSLEDGDRCKAKMEKQASSFDFNGP
YLGPPHSRSLPDILGQPEPPQEGGSQKSPPPGSLEYLCLPAGGQVQLVPLAQDTEDPGVAS
GYVSSADLVFTPNSGASSVSGPPGAPGPVKSGFEGYVELPPIEGRSPRSQPEGLLVLQQVG
DYCFLPGLGPGPLSLRSKQVPVIQLFKALKQQDYLSLPPWEVNKPGEVC
SEQ ID NO: 73 (BM NKG2D-IL12Rβ2-IL15Rα amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLSDPK
PENPACPWTVLPAGDLPTHDGYLPSNIDDLPSHEAPLADSLEELEPQKSRQTPPLASVEME
AMEALPVTWGTSSRDEDLENCSHHL
SEQ ID NO: 74 (BM NKG2D-IL12Rβ2-IL15Rβ-1 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLSDPK
PENPACPWTVLPAGDLPTHDGYLPSNIDDLPSHEAPLADSLEELEPQVTQLLLQQDKVPE
PASLSSNHSLTSCFTNQGYFFFHLPDALEIEACQVYFTYDPYSEEDPDEGVAGAPTGSSPQ
PLQPLSGEDDAYCTFPSRDDLLLFSPSLLGGPSPPSTAPGGSGAGEERMPPSLQERVPRDW
DPQPLGPPTPGVPDLVDFQPPPELVLREAGEEVPDAGPREGVSFPWSRPPGQGEFRALNA
RLPLNTDAYLSLQELQGQDPTHLV
SEQ ID NO: 75 (BM NKG2D-IL12Rβ2-IL15Rβ-2 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLSDPK
PENPACPWTVLPAGDLPTHDGYLPSNIDDLPSHEAPLADSLEELEPQEPASLSSNHSLTSCF
TNQGYFFFHLPDALEIEACQVYFTYDPYSEEDPDEGVAGAPTGSSPQPLQPLSGEDDAYC
TFPSRDDLLLFSEFRALNARLPLNTDAYLSLQELQGQDPTHLV
SEQ ID NO: 76 (BM NKG2D-IL12Rβ2-IL-21R-1 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLSDPK
PENPACPWTVLPAGDLPTHDGYLPSNIDDLPSHEAPLADSLEELEPQLELGPWSPEVPSTL
EVYSCHPPRSPAKRLQLTELQEPAELVESDGVPKPSFWPTAQNSGGSAYSEERDRPYGLVS
IDTVTVLDAEGPCTWPCSCEDDGYPALDLDAGLEPS
SEQ ID NO: 77 (BM NKG2D-IL12Rβ2-IL-21R-2 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLSDPK
PENPACPWTVLPAGDLPTHDGYLPSNIDDLPSHEAPLADSLEELEPQECDFTSPGDEGPPR
SYLRQWVVIPPPLSSPGPQAS
SEQ ID NO: 78 (BM NKG2D-IL12Rβ2-IL-21R-3 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLSDPK
PENPACPWTVLPAGDLPTHDGYLPSNIDDLPSHEAPLADSLEELEPQLELGPWSPEVPSTL
EVYSCHPPRSPAKRLQLTELQEPAELVESDGVPKPSFWPTAQNSGGSAYSEERDRPYGLVS
IDTVTVLDAEGPCTWPCSCEDDGYPALDLDAGLEPSECDFTSPGDEGPPRSYLRQWVVIP
PPLSSPGPQAS
SEQ ID NO: 79 (BM NKG2D-IL12Rβ2-IL-23R-2 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLSDPK
PENPACPWTVLPAGDLPTHDGYLPSNIDDLPSHEAPLADSLEELEPQEFVSCLGIVNEELP
SINTYFPQNILESHENRIS
SEQ ID NO: 80 (BM NKG2D-IL12Rβ2-GM-CSFRα amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLSDPK
PENPACPWTVLPAGDLPTHDGYLPSNIDDLPSHEAPLADSLEELEPQEVEDEIIWEEFTPEE
GKGYREEVLTVKEIT
SEQ ID NO: 81 (BM NKG2D-IL12Rβ2-GM-CSFRβ-1 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLSDPK
PENPACPWTVLPAGDLPTHDGYLPSNIDDLPSHEAPLADSLEELEPQDTEDPGVASGYVSS
ADLVFTPNSGASSVSGPPGAPGPVKSGFEGYVELPPIEGRSPRSQPEGLLVLQQVGDYCFL
PGLGPGPLSLRSKQVPVIQLFKALKQQDYLSLPPWEVNKPGEVC
SEQ ID NO: 82 (BM NKG2D-IL12Rβ2-GM-CSFRβ-2 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLSDPK
PENPACPWTVLPAGDLPTHDGYLPSNIDDLPSHEAPLADSLEELEPQEKQASSFDFNGPYL
GPPHSRSLPDILGQPEPPQEGGSQKSPPPGSLEYLCLPAGGQVQLVPLAQDTEDPGVASGY
VSSADLVFTPNSGASSVSGPPGAPGPVKSGFEGYVELPPIEGRSPRSQPEGLLVLQQVGDY
CFLPGLGPGPLSLRSKQVPVIQLFKALKQQDYLSLPPWEVNKPGEVC
SEQ ID NO: 83 (BM NKG2D-IL15Rα-IL-21R-1 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLKSR
QTPPLASVEMEAMEALPVTWGTSSRDEDLENCSHHLLELGPWSPEVPSTLEVYSCHPPR
SPAKRLQLTELQEPAELVESDGVPKPSFWPTAQNSGGSAYSEERDRPYGLVSIDTVTVLDA
EGPCTWPCSCEDDGYPALDLDAGLEPS
SEQ ID NO: 84 (BM NKG2D-IL15Rα-IL-21R-2 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLKSR
QTPPLASVEMEAMEALPVTWGTSSRDEDLENCSHHLECDFTSPGDEGPPRSYLRQWVVI
PPPLSSPGPQAS
SEQ ID NO: 85 (BM NKG2D-IL15Rα-IL-21R-3 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLKSR
QTPPLASVEMEAMEALPVTWGTSSRDEDLENCSHHLLELGPWSPEVPSTLEVYSCHPPR
SPAKRLQLTELQEPAELVESDGVPKPSFWPTAQNSGGSAYSEERDRPYGLVSIDTVTVLDA
EGPCTWPCSCEDDGYPALDLDAGLEPSECDFTSPGDEGPPRSYLRQWVVIPPPLSSPGPQ
AS
SEQ ID NO: 86 (BM NKG2D-IL15Rα-IL-23R-2 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLKSR
QTPPLASVEMEAMEALPVTWGTSSRDEDLENCSHHLEFVSCLGIVNEELPSINTYFPQNIL
ESHENRIS
SEQ ID NO: 87 (BM NKG2D-IL15Rα-GM-CSFRα amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLKSR
QTPPLASVEMEAMEALPVTWGTSSRDEDLENCSHHLEVEDEIIWEEFTPEEGKGYREEV
LTVKEIT
SEQ ID NO: 88 (BM NKG2D-IL15Rα-GM-CSFRβ-1 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLKSR
QTPPLASVEMEAMEALPVTWGTSSRDEDLENCSHHLDTEDPGVASGYVSSADLVFTPNS
GASSVSGPPGAPGPVKSGFEGYVELPPIEGRSPRSQPEGLLVLQQVGDYCFLPGLGPGPLS
LRSKQVPVIQLFKALKQQDYLSLPPWEVNKPGEVC
SEQ ID NO: 89 (BM NKG2D-IL15Rα-GM-CSFRβ-2 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLKSR
QTPPLASVEMEAMEALPVTWGTSSRDEDLENCSHHLEKQASSFDFNGPYLGPPHSRSLP
DILGQPEPPQEGGSQKSPPPGSLEYLCLPAGGQVQLVPLAQDTEDPGVASGYVSSADLVFT
PNSGASSVSGPPGAPGPVKSGFEGYVELPPIEGRSPRSQPEGLLVLQQVGDYCFLPGLGPG
PLSLRSKQVPVIQLFKALKQQDYLSLPPWEVNKPGEVC
SEQ ID NO: 90 (BM NKG2D-IL15Rβ-1-IL-21R-1 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLVTQL
LLQQDKVPEPASLSSNHSLTSCFTNQGYFFFHLPDALEIEACQVYFTYDPYSEEDPDEGVA
GAPTGSSPQPLQPLSGEDDAYCTFPSRDDLLLFSPSLLGGPSPPSTAPGGSGAGEERMPPSL
QERVPRDWDPQPLGPPTPGVPDLVDFQPPPELVLREAGEEVPDAGPREGVSFPWSRPPGQ
GEFRALNARLPLNTDAYLSLQELQGQDPTHLVLELGPWSPEVPSTLEVYSCHPPRSPAKR
LQLTELQEPAELVESDGVPKPSFWPTAQNSGGSAYSEERDRPYGLVSIDTVTVLDAEGPCT
WPCSCEDDGYPALDLDAGLEPS
SEQ ID NO: 91 (BM NKG2D-IL15Rβ-1-IL-21R-2 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLVTQL
LLQQDKVPEPASLSSNHSLTSCFTNQGYFFFHLPDALEIEACQVYFTYDPYSEEDPDEGVA
GAPTGSSPQPLQPLSGEDDAYCTFPSRDDLLLFSPSLLGGPSPPSTAPGGSGAGEERMPPSL
QERVPRDWDPQPLGPPTPGVPDLVDFQPPPELVLREAGEEVPDAGPREGVSFPWSRPPGQ
GEFRALNARLPLNTDAYLSLQELQGQDPTHLVECDFTSPGDEGPPRSYLRQWVVIPPPLS
SPGPQAS
SEQ ID NO: 92 (BM NKG2D-IL15Rβ-1-IL-21R-3 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLVTQL
LLQQDKVPEPASLSSNHSLTSCFTNQGYFFFHLPDALEIEACQVYFTYDPYSEEDPDEGVA
GAPTGSSPQPLQPLSGEDDAYCTFPSRDDLLLFSPSLLGGPSPPSTAPGGSGAGEERMPPSL
QERVPRDWDPQPLGPPTPGVPDLVDFQPPPELVLREAGEEVPDAGPREGVSFPWSRPPGQ
GEFRALNARLPLNTDAYLSLQELQGQDPTHLVLELGPWSPEVPSTLEVYSCHPPRSPAKR
LQLTELQEPAELVESDGVPKPSFWPTAQNSGGSAYSEERDRPYGLVSIDTVTVLDAEGPCT
WPCSCEDDGYPALDLDAGLEPSECDFTSPGDEGPPRSYLRQWVVIPPPLSSPGPQAS
SEQ ID NO: 93 (BM NKG2D-IL15Rβ-1-IL-23R-2 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLVTQL
LLQQDKVPEPASLSSNHSLTSCFTNQGYFFFHLPDALEIEACQVYFTYDPYSEEDPDEGVA
GAPTGSSPQPLQPLSGEDDAYCTFPSRDDLLLFSPSLLGGPSPPSTAPGGSGAGEERMPPSL
QERVPRDWDPQPLGPPTPGVPDLVDFQPPPELVLREAGEEVPDAGPREGVSFPWSRPPGQ
GEFRALNARLPLNTDAYLSLQELQGQDPTHLVEFVSCLGIVNEELPSINTYFPQNILESHF
NRIS
SEQ ID NO: 94 (BM NKG2D-IL15Rβ-1-GM-CSFRα amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLVTQL
LLQQDKVPEPASLSSNHSLTSCFTNQGYFFFHLPDALEIEACQVYFTYDPYSEEDPDEGVA
GAPTGSSPQPLQPLSGEDDAYCTFPSRDDLLLFSPSLLGGPSPPSTAPGGSGAGEERMPPSL
QERVPRDWDPQPLGPPTPGVPDLVDFQPPPELVLREAGEEVPDAGPREGVSFPWSRPPGQ
GEFRALNARLPLNTDAYLSLQELQGQDPTHLVEVEDEIIWEEFTPEEGKGYREEVLTVKEI
T
SEQ ID NO: 95 (BM NKG2D-IL15Rβ-1-GM-CSFRβ-1 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLVTQL
LLQQDKVPEPASLSSNHSLTSCFTNQGYFFFHLPDALEIEACQVYFTYDPYSEEDPDEGVA
GAPTGSSPQPLQPLSGEDDAYCTFPSRDDLLLFSPSLLGGPSPPSTAPGGSGAGEERMPPSL
QERVPRDWDPQPLGPPTPGVPDLVDFQPPPELVLREAGEEVPDAGPREGVSFPWSRPPGQ
GEFRALNARLPLNTDAYLSLQELQGQDPTHLVDTEDPGVASGYVSSADLVFTPNSGASSV
SGPPGAPGPVKSGFEGYVELPPIEGRSPRSQPEGLLVLQQVGDYCFLPGLGPGPLSLRSKQ
VPVIQLFKALKQQDYLSLPPWEVNKPGEVC
SEQ ID NO: 96 (BM NKG2D-IL15Rβ-1-GM-CSFRβ-2 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLVTQL
LLQQDKVPEPASLSSNHSLTSCFTNQGYFFFHLPDALEIEACQVYFTYDPYSEEDPDEGVA
GAPTGSSPQPLQPLSGEDDAYCTFPSRDDLLLFSPSLLGGPSPPSTAPGGSGAGEERMPPSL
QERVPRDWDPQPLGPPTPGVPDLVDFQPPPELVLREAGEEVPDAGPREGVSFPWSRPPGQ
GEFRALNARLPLNTDAYLSLQELQGQDPTHLVEKQASSFDFNGPYLGPPHSRSLPDILGQ
PEPPQEGGSQKSPPPGSLEYLCLPAGGQVQLVPLAQDTEDPGVASGYVSSADLVFTPNSG
ASSVSGPPGAPGPVKSGFEGYVELPPIEGRSPRSQPEGLLVLQQVGDYCFLPGLGPGPLSL
RSKQVPVIQLFKALKQQDYLSLPPWEVNKPGEVC
SEQ ID NO: 97 (BM NKG2D-IL15Rβ-2-IL-21R-1 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLEPAS
LSSNHSLTSCFTNQGYFFFHLPDALEIEACQVYFTYDPYSEEDPDEGVAGAPTGSSPQPLQ
PLSGEDDAYCTFPSRDDLLLFSEFRALNARLPLNTDAYLSLQELQGQDPTHLVLELGPWS
PEVPSTLEVYSCHPPRSPAKRLQLTELQEPAELVESDGVPKPSFWPTAQNSGGSAYSEERD
RPYGLVSIDTVTVLDAEGPCTWPCSCEDDGYPALDLDAGLEPS
SEQ ID NO: 98 (BM NKG2D-IL15Rβ-2-IL-21R-2 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLEPAS
LSSNHSLTSCFTNQGYFFFHLPDALEIEACQVYFTYDPYSEEDPDEGVAGAPTGSSPQPLQ
PLSGEDDAYCTFPSRDDLLLFSEFRALNARLPLNTDAYLSLQELQGQDPTHLVECDFTSPG
DEGPPRSYLRQWVVIPPPLSSPGPQAS
SEQ ID NO: 99 (BM NKG2D-IL15Rβ-2-IL-21R-3 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLEPAS
LSSNHSLTSCFTNQGYFFFHLPDALEIEACQVYFTYDPYSEEDPDEGVAGAPTGSSPQPLQ
PLSGEDDAYCTFPSRDDLLLFSEFRALNARLPLNTDAYLSLQELQGQDPTHLVLELGPWS
PEVPSTLEVYSCHPPRSPAKRLQLTELQEPAELVESDGVPKPSFWPTAQNSGGSAYSEERD
RPYGLVSIDTVTVLDAEGPCTWPCSCEDDGYPALDLDAGLEPSECDFTSPGDEGPPRSYL
RQWVVIPPPLSSPGPQAS
SEQ ID NO: 100 (BM NKG2D-IL15Rβ-2-IL-23R-2 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLEPAS
LSSNHSLTSCFTNQGYFFFHLPDALEIEACQVYFTYDPYSEEDPDEGVAGAPTGSSPQPLQ
PLSGEDDAYCTFPSRDDLLLFSEFRALNARLPLNTDAYLSLQELQGQDPTHLVEFVSCLGI
VNEELPSINTYFPQNILESHENRIS
SEQ ID NO: 101 (BM NKG2D-IL15Rβ-2-GM-CSFRα amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLEPAS
LSSNHSLTSCFTNQGYFFFHLPDALEIEACQVYFTYDPYSEEDPDEGVAGAPTGSSPQPLQ
PLSGEDDAYCTFPSRDDLLLFSEFRALNARLPLNTDAYLSLQELQGQDPTHLVEVEDEIIW
EEFTPEEGKGYREEVLTVKEIT
SEQ ID NO: 102 (BM NKG2D-IL15Rβ-2-GM-CSFRβ-1 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLEPAS
LSSNHSLTSCFTNQGYFFFHLPDALEIEACQVYFTYDPYSEEDPDEGVAGAPTGSSPQPLQ
PLSGEDDAYCTFPSRDDLLLFSEFRALNARLPLNTDAYLSLQELQGQDPTHLVDTEDPGV
ASGYVSSADLVFTPNSGASSVSGPPGAPGPVKSGFEGYVELPPIEGRSPRSQPEGLLVLQQ
VGDYCFLPGLGPGPLSLRSKQVPVIQLFKALKQQDYLSLPPWEVNKPGEVC
SEQ ID NO: 103 (BM NKG2D-IL15Rβ-2-GM-CSFRβ-2 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLEPAS
LSSNHSLTSCFTNQGYFFFHLPDALEIEACQVYFTYDPYSEEDPDEGVAGAPTGSSPQPLQ
PLSGEDDAYCTFPSRDDLLLFSEFRALNARLPLNTDAYLSLQELQGQDPTHLVEKQASSF
DFNGPYLGPPHSRSLPDILGQPEPPQEGGSQKSPPPGSLEYLCLPAGGQVQLVPLAQDTED
PGVASGYVSSADLVFTPNSGASSVSGPPGAPGPVKSGFEGYVELPPIEGRSPRSQPEGLLV
LQQVGDYCFLPGLGPGPLSLRSKQVPVIQLFKALKQQDYLSLPPWEVNKPGEVC
SEQ ID NO: 104 (BM NKG2D-IL-21R-1-IL-23R-2 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLLELG
PWSPEVPSTLEVYSCHPPRSPAKRLQLTELQEPAELVESDGVPKPSFWPTAQNSGGSAYSE
ERDRPYGLVSIDTVTVLDAEGPCTWPCSCEDDGYPALDLDAGLEPSEFVSCLGIVNEELPS
INTYFPQNILESHENRIS
SEQ ID NO: 105 (BM NKG2D-IL-21R-1-GM-CSFRα amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLLELG
PWSPEVPSTLEVYSCHPPRSPAKRLQLTELQEPAELVESDGVPKPSFWPTAQNSGGSAYSE
ERDRPYGLVSIDTVTVLDAEGPCTWPCSCEDDGYPALDLDAGLEPSEVEDEIIWEEFTPEE
GKGYREEVLTVKEIT
SEQ ID NO: 106 (BM NKG2D-IL-21R-1-GM-CSFRβ-1 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLLELG
PWSPEVPSTLEVYSCHPPRSPAKRLQLTELQEPAELVESDGVPKPSFWPTAQNSGGSAYSE
ERDRPYGLVSIDTVTVLDAEGPCTWPCSCEDDGYPALDLDAGLEPSDTEDPGVASGYVSS
ADLVFTPNSGASSVSGPPGAPGPVKSGFEGYVELPPIEGRSPRSQPEGLLVLQQVGDYCFL
PGLGPGPLSLRSKQVPVIQLFKALKQQDYLSLPPWEVNKPGEVC
SEQ ID NO: 107 (BM NKG2D-IL-21R-1-GM-CSFRβ-2 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLLELG
PWSPEVPSTLEVYSCHPPRSPAKRLQLTELQEPAELVESDGVPKPSFWPTAQNSGGSAYSE
ERDRPYGLVSIDTVTVLDAEGPCTWPCSCEDDGYPALDLDAGLEPSEKQASSFDFNGPYL
GPPHSRSLPDILGQPEPPQEGGSQKSPPPGSLEYLCLPAGGQVQLVPLAQDTEDPGVASGY
VSSADLVFTPNSGASSVSGPPGAPGPVKSGFEGYVELPPIEGRSPRSQPEGLLVLQQVGDY
CFLPGLGPGPLSLRSKQVPVIQLFKALKQQDYLSLPPWEVNKPGEVC
SEQ ID NO: 108 (BM NKG2D-IL-21R-2-IL-23R-2 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLECDF
TSPGDEGPPRSYLRQWVVIPPPLSSPGPQASEFVSCLGIVNEELPSINTYFPQNILESHFNRI
S
SEQ ID NO: 109 (BM NKG2D-IL-21R-2-GM-CSFRα amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLECDF
TSPGDEGPPRSYLRQWVVIPPPLSSPGPQASEVEDEIIWEEFTPEEGKGYREEVLTVKEIT
SEQ ID NO: 110 (BM NKG2D-IL-21R-2-GM-CSFRβ-1 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLECDF
TSPGDEGPPRSYLRQWVVIPPPLSSPGPQASDTEDPGVASGYVSSADLVFTPNSGASSVSG
PPGAPGPVKSGFEGYVELPPIEGRSPRSQPEGLLVLQQVGDYCFLPGLGPGPLSLRSKQVP
VIQLFKALKQQDYLSLPPWEVNKPGEVC
SEQ ID NO: 111 (BM NKG2D-IL-21R-2-GM-CSFRβ-2 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLECDF
TSPGDEGPPRSYLRQWVVIPPPLSSPGPQASEKQASSFDFNGPYLGPPHSRSLPDILGQPEP
PQEGGSQKSPPPGSLEYLCLPAGGQVQLVPLAQDTEDPGVASGYVSSADLVFTPNSGASS
VSGPPGAPGPVKSGFEGYVELPPIEGRSPRSQPEGLLVLQQVGDYCFLPGLGPGPLSLRSK
QVPVIQLFKALKQQDYLSLPPWEVNKPGEVC
SEQ ID NO: 112 (BM NKG2D-IL-21R-3-IL-23R-2 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLLELG
PWSPEVPSTLEVYSCHPPRSPAKRLQLTELQEPAELVESDGVPKPSFWPTAQNSGGSAYSE
ERDRPYGLVSIDTVTVLDAEGPCTWPCSCEDDGYPALDLDAGLEPSECDFTSPGDEGPPR
SYLRQWVVIPPPLSSPGPQASEFVSCLGIVNEELPSINTYFPQNILESHENRIS
SEQ ID NO: 113 (BM NKG2D-IL-21R-3-GM-CSFRα amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLLELG
PWSPEVPSTLEVYSCHPPRSPAKRLQLTELQEPAELVESDGVPKPSFWPTAQNSGGSAYSE
ERDRPYGLVSIDTVTVLDAEGPCTWPCSCEDDGYPALDLDAGLEPSECDFTSPGDEGPPR
SYLRQWVVIPPPLSSPGPQASEVEDEIIWEEFTPEEGKGYREEVLTVKEIT
SEQ ID NO: 114 (BM NKG2D-IL-21R-3-GM-CSFRβ-1 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLLELG
PWSPEVPSTLEVYSCHPPRSPAKRLQLTELQEPAELVESDGVPKPSFWPTAQNSGGSAYSE
ERDRPYGLVSIDTVTVLDAEGPCTWPCSCEDDGYPALDLDAGLEPSECDFTSPGDEGPPR
SYLRQWVVIPPPLSSPGPQASDTEDPGVASGYVSSADLVFTPNSGASSVSGPPGAPGPVKS
GFEGYVELPPIEGRSPRSQPEGLLVLQQVGDYCFLPGLGPGPLSLRSKQVPVIQLFKALKQ
QDYLSLPPWEVNKPGEVC
SEQ ID NO: 115 (BM NKG2D-IL-21R-3-GM-CSFRβ-2 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLLELG
PWSPEVPSTLEVYSCHPPRSPAKRLQLTELQEPAELVESDGVPKPSFWPTAQNSGGSAYSE
ERDRPYGLVSIDTVTVLDAEGPCTWPCSCEDDGYPALDLDAGLEPSECDFTSPGDEGPPR
SYLRQWVVIPPPLSSPGPQASEKQASSFDFNGPYLGPPHSRSLPDILGQPEPPQEGGSQKSP
PPGSLEYLCLPAGGQVQLVPLAQDTEDPGVASGYVSSADLVFTPNSGASSVSGPPGAPGP
VKSGFEGYVELPPIEGRSPRSQPEGLLVLQQVGDYCFLPGLGPGPLSLRSKQVPVIQLFKA
LKQQDYLSLPPWEVNKPGEVC
SEQ ID NO: 116 (BM NKG2D-IL-23R-2-GM-CSFRα amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLEFVS
CLGIVNEELPSINTYFPQNILESHFNRISEVEDEIIWEEFTPEEGKGYREEVLTVKEIT
SEQ ID NO: 117 (BM NKG2D-IL-23R-2-GM-CSFRβ-1 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLEFVS
CLGIVNEELPSINTYFPQNILESHFNRISDTEDPGVASGYVSSADLVFTPNSGASSVSGPPG
APGPVKSGFEGYVELPPIEGRSPRSQPEGLLVLQQVGDYCFLPGLGPGPLSLRSKQVPVIQ
LFKALKQQDYLSLPPWEVNKPGEVC
SEQ ID NO: 118 (BM NKG2D-IL-23R-2-GM-CSFRβ-2 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLEFVS
CLGIVNEELPSINTYFPQNILESHENRISEKQASSFDFNGPYLGPPHSRSLPDILGQPEPPQE
GGSQKSPPPGSLEYLCLPAGGQVQLVPLAQDTEDPGVASGYVSSADLVFTPNSGASSVSG
PPGAPGPVKSGFEGYVELPPIEGRSPRSQPEGLLVLQQVGDYCFLPGLGPGPLSLRSKQVP
VIQLFKALKQQDYLSLPPWEVNKPGEVC
SEQ ID NO: 119 (BM NKG2D-IL-7Rα-IL-9R-2 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLARD
EVEGFLQDTFPQQLEESEKQRLGGDVQSPNCPSEDVVITPESFGRDSSLTCLAGNVSACD
APILSSSRSLDCRESGKNGPHVYQDLLLSLGTTNSTLPPPFSLQSGILTLNPVAQGQPILTSL
GSNQEEAYVTMSSFYQNQDVLPAGCTEWRVQTLAYLPQEDWAPTSLTRPAPPDSEGSRSS
SSSSSSNNNNYCALGCYGGWHLSALPGNTQSS
SEQ ID NO: 120 (BM NKG2D-IL12Rβ1-IL-9R-2 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLSLQE
ALVVEMSWDKGERTEPLEKTELPEGAPELALDTELSLEDGDRCKAKMDVLPAGCTEWR
VQTLAYLPQEDWAPTSLTRPAPPDSEGSRSSSSSSSSNNNNYCALGCYGGWHLSALPGNT
QSS
SEQ ID NO: 121 (BM NKG2D-IL12Rβ2-IL-9R-2 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLSDPK
PENPACPWTVLPAGDLPTHDGYLPSNIDDLPSHEAPLADSLEELEPQDVLPAGCTEWRVQ
TLAYLPQEDWAPTSLTRPAPPDSEGSRSSSSSSSSNNNNYCALGCYGGWHLSALPGNTQS
S
SEQ ID NO: 122 (BM NKG2D-IL15Rα-IL-9R-2 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLKSR
QTPPLASVEMEAMEALPVTWGTSSRDEDLENCSHHLDVLPAGCTEWRVQTLAYLPQED
WAPTSLTRPAPPDSEGSRSSSSSSSSNNNNYCALGCYGGWHLSALPGNTQSS
SEQ ID NO: 123 (BM NKG2D-IL15Rβ-1-IL-9R-2 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLVTQL
LLQQDKVPEPASLSSNHSLTSCFTNQGYFFFHLPDALEIEACQVYFTYDPYSEEDPDEGVA
GAPTGSSPQPLQPLSGEDDAYCTFPSRDDLLLFSPSLLGGPSPPSTAPGGSGAGEERMPPSL
QERVPRDWDPQPLGPPTPGVPDLVDFQPPPELVLREAGEEVPDAGPREGVSFPWSRPPGQ
GEFRALNARLPLNTDAYLSLQELQGQDPTHLVDVLPAGCTEWRVQTLAYLPQEDWAPTS
LTRPAPPDSEGSRSSSSSSSSNNNNYCALGCYGGWHLSALPGNTQSS
SEQ ID NO: 124 (BM NKG2D-IL15Rβ-2-IL-9R-2 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLEPAS
LSSNHSLTSCFTNQGYFFFHLPDALEIEACQVYFTYDPYSEEDPDEGVAGAPTGSSPQPLQ
PLSGEDDAYCTFPSRDDLLLFSEFRALNARLPLNTDAYLSLQELQGQDPTHLVDVLPAGC
TEWRVQTLAYLPQEDWAPTSLTRPAPPDSEGSRSSSSSSSSNNNNYCALGCYGGWHLSAL
PGNTQSS
SEQ ID NO: 125 (BM NKG2D-IL-21R-1-IL-9R-2 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLLELG
PWSPEVPSTLEVYSCHPPRSPAKRLQLTELQEPAELVESDGVPKPSFWPTAQNSGGSAYSE
ERDRPYGLVSIDTVTVLDAEGPCTWPCSCEDDGYPALDLDAGLEPSDVLPAGCTEWRVQ
TLAYLPQEDWAPTSLTRPAPPDSEGSRSSSSSSSSNNNNYCALGCYGGWHLSALPGNTQS
S
SEQ ID NO: 126 (BM NKG2D-IL-21R-2-IL-9R-2 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLECDF
TSPGDEGPPRSYLRQWVVIPPPLSSPGPQASDVLPAGCTEWRVQTLAYLPQEDWAPTSLT
RPAPPDSEGSRSSSSSSSSNNNNYCALGCYGGWHLSALPGNTQSS
SEQ ID NO: 127 (BM NKG2D-IL-21R-3-IL-9R-2 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLLELG
PWSPEVPSTLEVYSCHPPRSPAKRLQLTELQEPAELVESDGVPKPSFWPTAQNSGGSAYSE
ERDRPYGLVSIDTVTVLDAEGPCTWPCSCEDDGYPALDLDAGLEPSECDFTSPGDEGPPR
SYLRQWVVIPPPLSSPGPQASDVLPAGCTEWRVQTLAYLPQEDWAPTSLTRPAPPDSEGSR
SSSSSSSSNNNNYCALGCYGGWHLSALPGNTQSS
SEQ ID NO: 128 (BM NKG2D-IL-23R-2-IL-9R-2 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLEFVS
CLGIVNEELPSINTYFPQNILESHENRISDVLPAGCTEWRVQTLAYLPQEDWAPTSLTRPAP
PDSEGSRSSSSSSSSNNNNYCALGCYGGWHLSALPGNTQSS
SEQ ID NO: 129 (BM NKG2D-GM-CSFRα-IL-9R-2 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLEVE
DEITWEEFTPEEGKGYREEVLTVKEITDVLPAGCTEWRVQTLAYLPQEDWAPTSLTRPAPP
DSEGSRSSSSSSSSNNNNYCALGCYGGWHLSALPGNTQSS
SEQ ID NO: 130 (BM NKG2D-GM-CSFRβ-1-IL-9R-2 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLDTE
DPGVASGYVSSADLVFTPNSGASSVSGPPGAPGPVKSGFEGYVELPPIEGRSPRSQPEGLL
VLQQVGDYCFLPGLGPGPLSLRSKQVPVIQLFKALKQQDYLSLPPWEVNKPGEVCDVLP
AGCTEWRVQTLAYLPQEDWAPTSLTRPAPPDSEGSRSSSSSSSSNNNNYCALGCYGGWH
LSALPGNTQSS
SEQ ID NO: 131 (BM NKG2D-GM-CSFRβ-2-IL-9R-2 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLEKQ
ASSFDFNGPYLGPPHSRSLPDILGQPEPPQEGGSQKSPPPGSLEYLCLPAGGQVQLVPLAQ
DTEDPGVASGYVSSADLVFTPNSGASSVSGPPGAPGPVKSGFEGYVELPPIEGRSPRSQPE
GLLVLQQVGDYCFLPGLGPGPLSLRSKQVPVIQLFKALKQQDYLSLPPWEVNKPGEVCD
VLPAGCTEWRVQTLAYLPQEDWAPTSLTRPAPPDSEGSRSSSSSSSSNNNNYCALGCYGG
WHLSALPGNTQSS
SEQ ID NO: 132 (BM truncated NKG2D (89-216)-IL15Rβ-1-IL-23R-2 amino acid
sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPIPLT
ESYCGPCPKNWICYKNNCYQFFDESKNWYESQASCMSQNASLLKVYSKEDQDLLKLVK
SYHWMGLVHIPTNGSWQWEDGSILSPNLLTIIEMQKGDCALYASSFKGYIENCSTPNTYIC
MQRTVSDPTRVETATETAWISLVTALHLVLGLSAVLGLLLLRWQFPAHYRRLRHALWPSL
PDLHRVLGQYLRDTAALSPPKATVSDTCEEVEPSLLEILPKSSERTPLPLVTQLLLQQDKV
PEPASLSSNHSLTSCFTNQGYFFFHLPDALEIEACQVYFTYDPYSEEDPDEGVAGAPTGSSP
QPLQPLSGEDDAYCTFPSRDDLLLFSPSLLGGPSPPSTAPGGSGAGEERMPPSLQERVPRD
WDPQPLGPPTPGVPDLVDFQPPPELVLREAGEEVPDAGPREGVSFPWSRPPGQGEFRALN
ARLPLNTDAYLSLQELQGQDPTHLVEFVSCLGIVNEELPSINTYFPQNILESHENRIS
SEQ ID NO: 133 (BM truncated NKG2D (98-216)-IL15Rβ-1-IL-23R-2 amino acid
sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPPCP
KNWICYKNNCYQFFDESKNWYESQASCMSQNASLLKVYSKEDQDLLKLVKSYHWMGL
VHIPTNGSWQWEDGSILSPNLLTIIEMQKGDCALYASSFKGYIENCSTPNTYICMQRTVSD
PTRVETATETAWISLVTALHLVLGLSAVLGLLLLRWQFPAHYRRLRHALWPSLPDLHRVLG
QYLRDTAALSPPKATVSDTCEEVEPSLLEILPKSSERTPLPLVTQLLLQQDKVPEPASLSSN
HSLTSCFTNQGYFFFHLPDALEIEACQVYFTYDPYSEEDPDEGVAGAPTGSSPQPLQPLSG
EDDAYCTFPSRDDLLLFSPSLLGGPSPPSTAPGGSGAGEERMPPSLQERVPRDWDPQPLGP
PTPGVPDLVDFQPPPELVLREAGEEVPDAGPREGVSFPWSRPPGQGEFRALNARLPLNTD
AYLSLQELQGQDPTHLVEFVSCLGIVNEELPSINTYFPQNILESHENRIS
SEQ ID NO: 134 (Anti-MIC-A/B scFv-IL7Rα-IL12Rβ2 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPQIQ
LVQSGPELKKPGETVKVSCKASGYMFTNYAMNWVKQAPEKGLKWMGWINTHTGDPT
YADDFKGRIAFSLETSASTAYLQINNLKNEDTATYFCVRTYGNYAMDYWGQGTSVTVSS
GGGGSGGGGSGGGGSDIQMTQTTSSLSASLGDRVTISCSASQDISNYLNWYQQKPDGTV
KLLIYDTSILHLGVPSRFSGSGSGTDYSLTISNLEPEDIATYYCQQYSKFPRTFGGGTTLEIK
SDPTRVETATETAWISLVTALHLVLGLSAVLGLLLLRWQFPAHYRRLRHALWPSLPDLHRV
LGQYLRDTAALSPPKATVSDTCEEVEPSLLEILPKSSERTPLPLARDEVEGFLQDTFPQQLE
ESEKQRLGGDVQSPNCPSEDVVITPESFGRDSSLTCLAGNVSACDAPILSSSRSLDCRESG
KNGPHVYQDLLLSLGTTNSTLPPPFSLQSGILTLNPVAQGQPILTSLGSNQEEAYVTMSSFY
QNQSDPKPENPACPWTVLPAGDLPTHDGYLPSNIDDLPSHEAPLADSLEELEPQ
SEQ ID NO: 135 (Construct 185 amino acid sequence)
MALPVTALLLPLALLLHAARPDIVMTQTPLSLSVTPGQPASISCRSSQSLVHSNGNTYLH
WYLQKPGQSPQLLIYKVSNRFSGVPDRFSGSGSGTDFTLKISRVEAEDVGVYYCSQNTH
VPPTFGQGTKVEIKGGGGSGGGGSGGGGSQVQLVQSGAEVKKPGASVKVSCKASGYTF
TDYEMHWVRQAPGQGLEWMGALDPKTGDTAYSQKFKGRVTLTADTSTSTVYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPR
SEQ ID NO: 136 (Construct 185-NKG2D-IL15Rβ-1-IL-21R-1 amino acid sequence)
MALPVTALLLPLALLLHAARPDIVMTQTPLSLSVTPGQPASISCRSSQSLVHSNGNTYLH
WYLQKPGQSPQLLIYKVSNRFSGVPDRFSGSGSGTDFTLKISRVEAEDVGVYYCSQNTH
VPPTFGQGTKVEIKGGGGSGGGGSGGGGSQVQLVQSGAEVKKPGASVKVSCKASGYTF
TDYEMHWVRQAPGQGLEWMGALDPKTGDTAYSQKFKGRVTLTADTSTSTVYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLVTQL
LLQQDKVPEPASLSSNHSLTSCFTNQGYFFFHLPDALEIEACQVYFTYDPYSEEDPDEGVA
GAPTGSSPQPLQPLSGEDDAYCTFPSRDDLLLFSPSLLGGPSPPSTAPGGSGAGEERMPPSL
QERVPRDWDPQPLGPPTPGVPDLVDFQPPPELVLREAGEEVPDAGPREGVSFPWSRPPGQ
GEFRALNARLPLNTDAYLSLQELQGQDPTHLVLELGPWSPEVPSTLEVYSCHPPRSPAKR
LQLTELQEPAELVESDGVPKPSFWPTAQNSGGSAYSEERDRPYGLVSIDTVTVLDAEGPCT
WPCSCEDDGYPALDLDAGLEPS
SEQ ID NO: 137 (Construct 185-NKG2D-IL15Rβ-2-IL-21R-2 amino acid sequence)
MALPVTALLLPLALLLHAARPDIVMTQTPLSLSVTPGQPASISCRSSQSLVHSNGNTYLH
WYLQKPGQSPQLLIYKVSNRFSGVPDRFSGSGSGTDFTLKISRVEAEDVGVYYCSQNTH
VPPTFGQGTKVEIKGGGGSGGGGSGGGGSQVQLVQSGAEVKKPGASVKVSCKASGYTF
TDYEMHWVRQAPGQGLEWMGALDPKTGDTAYSQKFKGRVTLTADTSTSTVYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLEPAS
LSSNHSLTSCFTNQGYFFFHLPDALEIEACQVYFTYDPYSEEDPDEGVAGAPTGSSPQPLQ
PLSGEDDAYCTFPSRDDLLLFSEFRALNARLPLNTDAYLSLQELQGQDPTHLVECDFTSPG
DEGPPRSYLRQWVVIPPPLSSPGPQAS
SEQ ID NO: 138 (Construct 185-NKG2D-IL15Rβ-2-IL-23R-2 amino acid sequence)
MALPVTALLLPLALLLHAARPDIVMTQTPLSLSVTPGQPASISCRSSQSLVHSNGNTYLH
WYLQKPGQSPQLLIYKVSNRFSGVPDRFSGSGSGTDFTLKISRVEAEDVGVYYCSQNTH
VPPTFGQGTKVEIKGGGGSGGGGSGGGGSQVQLVQSGAEVKKPGASVKVSCKASGYTF
TDYEMHWVRQAPGQGLEWMGALDPKTGDTAYSQKFKGRVTLTADTSTSTVYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLEPAS
LSSNHSLTSCFTNQGYFFFHLPDALEIEACQVYFTYDPYSEEDPDEGVAGAPTGSSPQPLQ
PLSGEDDAYCTFPSRDDLLLFSEFRALNARLPLNTDAYLSLQELQGQDPTHLVEFVSCLGI
VNEELPSINTYFPQNILESHENRIS
SEQ ID NO: 139 (Construct 185-NKG2D-IL15Rβ-2-GM-CSFRα amino acid sequence)
MALPVTALLLPLALLLHAARPDIVMTQTPLSLSVTPGQPASISCRSSQSLVHSNGNTYLH
WYLQKPGQSPQLLIYKVSNRFSGVPDRFSGSGSGTDFTLKISRVEAEDVGVYYCSQNTH
VPPTFGQGTKVEIKGGGGSGGGGSGGGGSQVQLVQSGAEVKKPGASVKVSCKASGYTF
TDYEMHWVRQAPGQGLEWMGALDPKTGDTAYSQKFKGRVTLTADTSTSTVYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLEPAS
LSSNHSLTSCFTNQGYFFFHLPDALEIEACQVYFTYDPYSEEDPDEGVAGAPTGSSPQPLQ
PLSGEDDAYCTFPSRDDLLLFSEFRALNARLPLNTDAYLSLQELQGQDPTHLVEVEDEIIW
EEFTPEEGKGYREEVLTVKEIT
SEQ ID NO: 140 (Construct 185-NKG2D-IL15Rβ-1-IL-9R-2 amino acid sequence)
MALPVTALLLPLALLLHAARPDIVMTQTPLSLSVTPGQPASISCRSSQSLVHSNGNTYLH
WYLQKPGQSPQLLIYKVSNRFSGVPDRFSGSGSGTDFTLKISRVEAEDVGVYYCSQNTH
VPPTFGQGTKVEIKGGGGSGGGGSGGGGSQVQLVQSGAEVKKPGASVKVSCKASGYTF
TDYEMHWVRQAPGQGLEWMGALDPKTGDTAYSQKFKGRVTLTADTSTSTVYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLVTQL
LLQQDKVPEPASLSSNHSLTSCFTNQGYFFFHLPDALEIEACQVYFTYDPYSEEDPDEGVA
GAPTGSSPQPLQPLSGEDDAYCTFPSRDDLLLFSPSLLGGPSPPSTAPGGSGAGEERMPPSL
QERVPRDWDPQPLGPPTPGVPDLVDFQPPPELVLREAGEEVPDAGPREGVSFPWSRPPGQ
GEFRALNARLPLNTDAYLSLQELQGQDPTHLVDVLPAGCTEWRVQTLAYLPQEDWAPTS
LTRPAPPDSEGSRSSSSSSSSNNNNYCALGCYGGWHLSALPGNTQSS
SEQ ID NO: 141 (CTL-019 amino acid sequence)
MALPVTALLLPLALLLHAARPDIQMTQTTSSLSASLGDRVTISCRASQDISKYLNWYQQK
PDGTVKLLIYHTSRLHSGVPSRFSGSGSGTDYSLTISNLEQEDIATYFCQQGNTLPYTFGG
GTKLEITGGGGSGGGGSGGGGSEVKLQESGPGLVAPSQSLSVTCTVSGVSLPDYGVSWIR
QPPRKGLEWLGVIWGSETTYYNSALKSRLTIIKDNSKSQVFLKMNSLQTDDTAIYYCAKH
YYYGGSYAMDYWGQGTSVTVSSTTTPAPRPPTPAPTIASQPLSLRPEACRPAAGGAVHTR
GLDFACDIYIWAPLAGTCGVLLLSLVITLYCKRGRKKLLYIFKQPFMRPVQTTQEEDGCSC
RFPEEEEGGCELRVKFSRSADAPAYKQGQNQLYNELNLGRREEYDVLDKRRGRDPEMG
GKPRRKNPQEGLYNELQKDKMAEAYSEIGMKGERRRGKGHDGLYQGLSTATKDTYDAL
HMQALPPR
SEQ ID NO: 142 (CTL-019-NKG2D-IL-7Rα-IL-12Rβ2 amino acid sequence)
MALPVTALLLPLALLLHAARPDIQMTQTTSSLSASLGDRVTISCRASQDISKYLNWYQQK
PDGTVKLLIYHTSRLHSGVPSRFSGSGSGTDYSLTISNLEQEDIATYFCQQGNTLPYTFGG
GTKLEITGGGGSGGGGSGGGGSEVKLQESGPGLVAPSQSLSVTCTVSGVSLPDYGVSWIR
QPPRKGLEWLGVIWGSETTYYNSALKSRLTIIKDNSKSQVFLKMNSLQTDDTAIYYCAKH
YYYGGSYAMDYWGQGTSVTVSSTTTPAPRPPTPAPTIASQPLSLRPEACRPAAGGAVHTR
GLDFACDIYIWAPLAGTCGVLLLSLVITLYCKRGRKKLLYIFKQPFMRPVQTTQEEDGCSC
RFPEEEEGGCELRVKFSRSADAPAYKQGQNQLYNELNLGRREEYDVLDKRRGRDPEMG
GKPRRKNPQEGLYNELQKDKMAEAYSEIGMKGERRRGKGHDGLYQGLSTATKDTYDAL
HMQALPPRGSGATNFSLLKQAGDVEENPGPMALPVTALLLPLALLLHAARPSLFNQEVQI
PLTESYCGPCPKNWICYKNNCYQFFDESKNWYESQASCMSQNASLLKVYSKEDQDLLK
LVKSYHWMGLVHIPTNGSWQWEDGSILSPNLLTIIEMQKGDCALYASSFKGYIENCSTPN
TYICMQRTVSDPTRVETATETAWISLVTALHLVLGLSAVLGLLLLRWQFPAHYRRLRHAL
WPSLPDLHRVLGQYLRDTAALSPPKATVSDTCEEVEPSLLEILPKSSERTPLPLARDEVEG
FLQDTFPQQLEESEKQRLGGDVQSPNCPSEDVVITPESFGRDSSLTCLAGNVSACDAPILS
SSRSLDCRESGKNGPHVYQDLLLSLGTTNSTLPPPFSLQSGILTLNPVAQGQPILTSLGSNQ
EEAYVTMSSFYQNQSDPKPENPACPWTVLPAGDLPTHDGYLPSNIDDLPSHEAPLADSLE
ELEPQ
SEQ ID NO: 143 (AS67190 amino acid sequence)
MALPVTALLLPLALLLHAARPQVQLVESGGGLVQAGGSLRLSCAASGNVFRFNIMGWY
RQAPGNQRELVASIDDGGDRSYADSVEGRFTISRENGKKIMYLQMNSLKPEDTAVYYCA
AGLGTYLNGRVSMATNYWGQGTQVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPR
SEQ ID NO: 144 (AS67190 NKG2D-IL-7Rα-IL-12Rβ2 amino acid sequence)
MALPVTALLLPLALLLHAARPQVQLVESGGGLVQAGGSLRLSCAASGNVFRFNIMGWY
RQAPGNQRELVASIDDGGDRSYADSVEGRFTISRENGKKIMYLQMNSLKPEDTAVYYCA
AGLGTYLNGRVSMATNYWGQGTQVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSLF
NQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIEN
CSTPNTYICMQRTVSDPTRVETATETAWISLVTALHLVLGLSAVLGLLLLRWQFPAHYRRL
RHALWPSLPDLHRVLGQYLRDTAALSPPKATVSDTCEEVEPSLLEILPKSSERTPLPLARD
EVEGFLQDTFPQQLEESEKQRLGGDVQSPNCPSEDVVITPESFGRDSSLTCLAGNVSACD
APILSSSRSLDCRESGKNGPHVYQDLLLSLGTTNSTLPPPFSLQSGILTLNPVAQGQPILTSL
GSNQEEAYVTMSSFYQNQSDPKPENPACPWTVLPAGDLPTHDGYLPSNIDDLPSHEAPLA
DSLEELEPQ
SEQ ID NO: 145 (TIGIT extracellular domain amino acid sequence)
MRWCLLLIWAQGLRQAPLASGMMTGTIETTGNISAEKGGSIILQCHLSSTTAQVTQVNW
EQQDQLLAICNADLGWHISPSFKDRVAPGPGLGLTLQSLTVNDTGEYFCIYHTYPDGTYT
GRIFLEVLESSVAEHGARFQIP
SEQ ID NO: 146 (BM TIGIT-IL-7Rα-IL-12Rβ2 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMRWCLLLIWAQGLRQAPLASGM
MTGTIETTGNISAEKGGSIILQCHLSSTTAQVTQVNWEQQDQLLAICNADLGWHISPSFK
DRVAPGPGLGLTLQSLTVNDTGEYFCIYHTYPDGTYTGRIFLEVLESSVAEHGARFQIPSDP
TRVETATETAWISLVTALHLVLGLSAVLGLLLLRWQFPAHYRRLRHALWPSLPDLHRVLG
QYLRDTAALSPPKATVSDTCEEVEPSLLEILPKSSERTPLPLARDEVEGFLQDTFPQQLEES
EKQRLGGDVQSPNCPSEDVVITPESFGRDSSLTCLAGNVSACDAPILSSSRSLDCRESGKN
GPHVYQDLLLSLGTTNSTLPPPFSLQSGILTLNPVAQGQPILTSLGSNQEEAYVTMSSFYQN
QSDPKPENPACPWTVLPAGDLPTHDGYLPSNIDDLPSHEAPLADSLEELEPQ
SEQ ID NO: 147 (SIRP-α extracellular domain amino acid sequence)
MEPAGPAPGRLGPLLCLLLAASCAWSGVAGEEELQVIQPDKSVLVAAGETATLRCTATSLI
PVGPIQWFRGAGPGRELIYNQKEGHFPRVTTVSDLTKRNNMDFSIRIGNITPADAGTYYC
VKFRKGSPDDVEFKSGAGTELSVRAKPSAPVVSGPAARATPQHTVSFTCESHGFSPRDITL
KWFKNGNELSDFQTNVDPVGESVSYSIHSTAKVVLTREDVHSQVICEVAHVTLQGDPLR
GTANLSETIRVPPTLEVTQQPVRAENQVNVTCQVRKFYPQRLQLTWLENGNVSRTETAST
VTENKDGTYNWMSWLLVNVSAHRDDVKLTCQVEHDGQPAVSKSHDLKVSAHPKEQGS
NTAAENTGSNERNIY
SEQ ID NO: 148 (AS67190 SIRP-α-IL-7Rα-IL-12Rβ2 amino acid sequence)
MALPVTALLLPLALLLHAARPQVQLVESGGGLVQAGGSLRLSCAASGNVFRFNIMGWY
RQAPGNQRELVASIDDGGDRSYADSVEGRFTISRENGKKIMYLQMNSLKPEDTAVYYCA
AGLGTYLNGRVSMATNYWGQGTQVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMEPAGPAPGRLGPLLCLLLAASCA
WSGVAGEEELQVIQPDKSVLVAAGETATLRCTATSLIPVGPIQWFRGAGPGRELIYNQKEG
HFPRVTTVSDLTKRNNMDFSIRIGNITPADAGTYYCVKFRKGSPDDVEFKSGAGTELSVR
AKPSAPVVSGPAARATPQHTVSFTCESHGFSPRDITLKWFKNGNELSDFQTNVDPVGESV
SYSIHSTAKVVLTREDVHSQVICEVAHVTLQGDPLRGTANLSETIRVPPTLEVTQQPVRAE
NQVNVTCQVRKFYPQRLQLTWLENGNVSRTETASTVTENKDGTYNWMSWLLVNVSAH
RDDVKLTCQVEHDGQPAVSKSHDLKVSAHPKEQGSNTAAENTGSNERNIYSDPTRVETA
TETAWISLVTALHLVLGLSAVLGLLLLRWQFPAHYRRLRHALWPSLPDLHRVLGQYLRDT
AALSPPKATVSDTCEEVEPSLLEILPKSSERTPLPLARDEVEGFLQDTFPQQLEESEKQRLG
GDVQSPNCPSEDVVITPESFGRDSSLTCLAGNVSACDAPILSSSRSLDCRESGKNGPHVYQ
DLLLSLGTTNSTLPPPFSLQSGILTLNPVAQGQPILTSLGSNQEEAYVTMSSFYQNQSDPKP
ENPACPWTVLPAGDLPTHDGYLPSNIDDLPSHEAPLADSLEELEPQ
SEQ ID NO: 149 (NKG2D CAR amino acid sequence)
MALPVTALLLPLALLLHAARPSLFNQEVQIPLTESYCGPCPKNWICYKNNCYQFFDESKN
WYESQASCMSQNASLLKVYSKEDQDLLKLVKSYHWMGLVHIPTNGSWQWEDGSILSPN
LLTIIEMQKGDCALYASSFKGYIENCSTPNTYICMQRTVTTTPAPRPPTPAPTIASQPLSLRP
EACRPAAGGAVHTRGLDFACDIYIWAPLAGTCGVLLLSLVITLYCKRGRKKLLYIFKQPF
MRPVQTTQEEDGCSCRFPEEEEGGCELRVKFSRSADAPAYKQGQNQLYNELNLGRREEY
DVLDKRRGRDPEMGGKPRRKNPQEGLYNELQKDKMAEAYSEIGMKGERRRGKGHDGL
YQGLSTATKDTYDALHMQALPPR
SEQ ID NO: 150 (BM NKG2D (Deleted) IL15Rβ-1-IL-21R-1 amino acid sequence)
MALPVTALLLPLALLLHAARPDVVMTQSPLSLPVTPGEPASISCRSSQSLVHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSSL
RSEDTAVYYCTRFYSYTYWGQGTLVTVSSTTTPAPRPPTPAPTIASQPLSLRPEACRPAAG
GAVHTRGLDFACDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKD
TYDALHMQALPPRGSGATNFSLLKQAGDVEENPGPMALPVTALLLPLALLLHAARPSDP
TRVETATETAWISLVTALHLVLGLSAVLGLLLLRWQFPAHYRRLRHALWPSLPDLHRVLG
QYLRDTAALSPPKATVSDTCEEVEPSLLEILPKSSERTPLPLVTQLLLQQDKVPEPASLSSN
HSLTSCFTNQGYFFFHLPDALEIEACQVYFTYDPYSEEDPDEGVAGAPTGSSPQPLQPLSG
EDDAYCTFPSRDDLLLFSPSLLGGPSPPSTAPGGSGAGEERMPPSLQERVPRDWDPQPLGP
PTPGVPDLVDFQPPPELVLREAGEEVPDAGPREGVSFPWSRPPGQGEFRALNARLPLNTD
AYLSLQELQGQDPTHLVLELGPWSPEVPSTLEVYSCHPPRSPAKRLQLTELQEPAELVESD
GVPKPSFWPTAQNSGGSAYSEERDRPYGLVSIDTVTVLDAEGPCTWPCSCEDDGYPALDL
DAGLEPS
SEQ ID NO: 151 (BM TPOR (S505N, W515K) IL15Rβ-1-IL-21R-1 amino acid sequence)
MALPVTALLLPLALLLHAARPDVVMTQSPLSLPVTPGEPASISCRSSQSL VHSNANTYLH
WYLQKPGQSPQLLIYKVSNRFSGVPDRFSGSGSGTDFTLKISRVEAEDVGVYYCSQNTH
VPPTFGQGTKLEIKRGGGGSGGGGSGGGGSQVQLVQSGAEVKKPGASVKVSCKASGYT
FTDYEMHWVRQAPGQGLEWMGALDPKTGDTAYSQKFKGRVTLTADESTSTAYMELSS
LRSEDTAVYYCTRFYSYTYWGQGTLVTVSSTTTPAPRPPTPAPTIASQPLSLRPEACRPAA
GGAVHTRGLDFACDIYIWAPLAGTCGVLLLSLVITLYCKRGRKKLLYIFKQPFMRPVQTT
QEEDGCSCRFPEEEEGGCELRVKFSRSADAPAYKQGQNQLYNELNLGRREEYDVLDKR
RGRDPEMGGKPRRKNPQEGLYNELQKDKMAEAYSEIGMKGERRRGKGHDGLYQGLST
ATKDTYDALHMQALPPRGSGATNFSLLKQAGDVEENPGPMALPVTALLLPLALLLHAA
RPSDPTRVETATETAWISLVTALHLVLGLNAVLGLLLLRKQFPAHYRRLRHALWPSLPD
LHRVLGQYLRDTAALSPPKATVSDTCEEVEPSLLEILPKSSERTPLPLVTQLLLQQDKVPE
PASLSSNHSLTSCFTNQGYFFFHLPDALEIEACQVYFTYDPYSEEDPDEGVAGAPTGSSPQ
PLQPLSGEDDAYCTFPSRDDLLLFSPSLLGGPSPPSTAPGGSGAGEERMPPSLQERVPRD
WDPQPLGPPTPGVPDLVDFQPPPELVLREAGEEVPDAGPREGVSFPWSRPPGQGEFRALN
ARLPLNTDAYLSLQELQGQDPTHLVLELGPWSPEVPSTLEVYSCHPPRSPAKRLQLTELQ
EPAELVESDGVPKPSFWPTAQNSGGSAYSEERDRPYGLVSIDTVTVLDAEGPCTWPCSCE
DDGYPALDLDAGLEPS
SEQ ID NO: 152 (Exemplary linker, n is an integer of at least one)
(GS)n
SEQ ID NO: 153 (Exemplary linker, n is an integer of at least one)
(GSGGS)n
SEQ ID NO: 154 (Exemplary linker, n is an integer of at least one)
(GGGS)n
SEQ ID NO: 155 (Exemplary linker, n is an integer of at least one)
(GGGGS)n
SEQ ID NO: 156 (truncated NKG2D (98-216))
PCPKNWICYKNNCYQFFDESKNWYESQASCMSQNASLLKVYSKEDQDLLKLVKSYHW
MGLVHIPTNGSWQWEDGSILSPNLLTIIEMQKGDCALYASSFKGYIENCSTPNTYICMQR
TV
SEQ ID NO: 157 (truncated NKG2D (89-216))
IPLTESYCGPCPKNWICYKNNCYQFFDESKNWYESQASCMSQNASLLKVYSKEDQDLLK
LVKSYHWMGLVHIPTNGSWQWEDGSILSPNLLTIIEMQKGDCALYASSFKGYIENCSTP
NTYICMQRTV
SEQ ID NO: 158 (C-MYC tag)
DEQKLISEEDL
SEQ ID NO: 159 (C-MYC-C-MYC tag)
DEQKLISEEDLEQKLISEEDL
SEQ ID NO: 160 (SBP tag)
DYMDEKTTGWRGGHVVEGLAGELEQLRARLEHHPQGQREP

What is claimed:

1. An immune effector cell expressing
(i) a chimeric cytokine receptor comprising:
(a) a first extracellular antigen binding domain, wherein the first extracellular antigen binding domain is derived from NKG2D, truncated NKG2D, antibody or antigen binding fragment thereof targeting NKG2D ligands, TIGIT or SIRP-α, or a variant thereof,
(b) a first transmembrane domain, and
(c) a cytokine receptor intracellular domain; and
(ii) optionally a functional exogenous receptor comprising:
(a) a second extracellular antigen binding domain,
(b) a second transmembrane domain, and
(c) an intracellular signaling domain.

2. The immune effector cell of claim 1, wherein the first extracellular antigen binding domain binds to an antigen expressed on the surface of a tumor cell.

3. The immune effector cell of claim 1 or claim 2,
(i) wherein the first extracellular antigen binding domain is derived from NKG2D or truncated NKG2D, or a variant thereof, wherein optionally the first extracellular antigen binding domain is derived from the extracellular domain (ECD) of NKG2D or truncated NKG2D, and wherein optionally the first extracellular antigen binding domain comprises the amino acid sequence of SEQ ID NOs: 6, 156 or 157, or an amino acid sequence having at least 75%, 80%, 85%, 90%, 91%, 92%, 93%, 94%, 95%, 96%, 97%, 98%, or 99% identity to SEQ ID NOs: 6, 156 or 157;
(ii) wherein the first extracelluar antigen domain comprises an antibody or antigen binding fragment thereof that binds to an NKG2D ligand or a variant thereof, wherein optionally the NKG2D ligand is selected from a group consisting of MICA/B, ULBP-1, ULBP-2,5,6 and ULBP-3, and wherein optionally the antibody or antigen binding fragment thereof comprises one or more scFv(s) or one or more sdAb(s) that bind to the NKG2D ligand;
(iii) wherein the first extracelluar antigen domain is derived from TIGIT or a variant thereof, wherein optionally the first extracelluar antigen domain is derived from the ECD of TIGIT, and wherein optionally the first extracellular antigen binding domain comprises the amino acid sequence of SEQ ID NO: 145 or an amino acid sequence having at least 75%, 80%, 85%, 90%, 91%, 92%, 93%, 94% 95% 96%, 97% 98%, or 99% identity to SEQ ID NO: 145; or
(iv) wherein the first extracelluar antigen domain is derived from SIRP-α or a variant thereof, wherein optionally the first extracelluar antigen domain is derived from the ECD of SIRP-α, and wherein optionally the first extracellular antigen binding domain comprises the amino acid sequence of SEQ ID NO: 147 or an amino acid sequence having at least 75%, 80%, 85%, 90%, 91%, 92%, 93%, 94%, 95%, 96%, 97%, 98%, or 99% identity to SEQ ID NO: 147.

4. The immune effector cell of any one of claims 1 to 3, wherein the first transmembrane domain comprises a Janus Kinase (JAK)-binding domain.

5. The immune effector cell of claim 4, wherein the JAK-binding domain is derived from a group consisting of EPOR, GHR, TPOR, or a variant thereof.

6. The immune effector cell of claim 5, wherein the JAK-binding domain is derived from TPOR and comprises an amino acid sequence of SEQ ID NO: 7 or an amino acid sequence having at least 75%, 80%, 85%, 90%, 91%, 92%, 93%, 94%, 95%, 96%, 97%, 98%, or 99% identity to SEQ ID NO: 7.

7. The immune effector cell of any one of claims 1 to 6, wherein the cytokine receptor intracellular domain is derived from one or more cytokine receptors selected from a group consisting of IL2Rα, IL7Rα, IL9R-1, IL9R-2, IL9R-3, IL12Rβ1, IL12Rβ2, IL15Rα, IL15Rβ-1, IL15Rβ-2, IL15Rβ-3, IL18Rα, IL18Rβ, IL21R-1, IL21R-2, IL21R-3, IL10R2, IL22R1, IL23R-1, IL23R-2, IL23R-3, IL27Rα, gp130, IL31RA, OSMRβ, IL36R, IL1RAcP, GM-CSFRα, GM-CSFRβ-1, GM-CSFRβ-2, or a variant thereof, and combinations thereof.

8. The immune effector cell of claim 7, wherein the cytokine receptor intracellular domain is selected from IL-7Rα, IL12Rβ1, IL12Rβ2, IL15Rα, IL15Rβ-1, IL15Rβ-2, IL15Rβ-3, IL-18Rα, IL18Rβ, IL21R-1, IL21R-2, IL21R-3, IL23R-1, IL23R-2, IL23R-3, GM-CSFRα, GM-CSFRβ-1, GM-CSFRβ-2, IL9R-1, TL9R-2, TL9R-3, IL-7Rα-IL12Rβ1, IL-7Rα-IL15Rα, IL-7Rα-IL15Rβ-1, IL-7Rα-IL15Rβ-2, IL-7Rα-IL21R-1, IL-7Rα-IL21R-2, IL-7Rα-IL21R-3, IL-7Rα-IL23R-2, IL-7Rα-GMCSFRα, IL-7Rα-GM-CSFRβ-1, IL-7Rα-GM-CSFRβ-2, IL12Rβ1-IL15Rα, IL12Rβ1-IL15Rβ-1, IL12Rβ1-IL15Rβ-2, IL12Rβ1-IL-21R-1, IL12Rβ1-IL-21R-2, IL12Rβ1-IL-21R-3, IL12Rβ1-IL-23R-2, IL12Rβ1-GM-CSFRα, IL12Rβ1-GM-CSFRβ-1, IL12Rβ1-GM-CSFRβ-2, IL12Rβ2-IL15Rα, IL12Rβ2-IL15Rβ-1, IL12Rβ2-IL15Rβ-2, IL12Rβ2-IL-21R-1, IL12Rβ2-IL-21R-2, IL12Rβ2-IL-21R-3, IL12Rβ2-IL-23R-2, IL12Rβ2-GM-CSFRα, IL12Rβ2-GM-CSFRβ-1, IL12Rβ2-GM-CSFRβ-2, IL15Rα-IL-21R-1, IL15Rα-IL-21R-2, IL15Rα-IL-21R-3, IL15Rα-IL-23R-2, IL15Rα-GM-CSFRα, IL15Rα-GM-CSFRβ-1, IL15Rα-GM-CSFRβ-2, IL15Rβ-1-IL-21R-1, L15Rβ-1-IL-21R-2, IL15Rβ-1-IL-21R-3, IL15Rβ-1-IL-23R-2, IL15Rβ-1-GM-CSFRα, IL15Rβ-1-GM-CSFRβ-1, IL15Rβ-1-GM-CSFRβ-2, IL15Rβ-2-IL-21R-1, IL15Rβ-2-IL-21R-2, IL15Rβ-2-IL-21R-3, L15Rβ-2-IL-23R-2, IL15Rβ-2-GM-CSFRα, IL15Rβ-2-GM-CSFRβ-1, IL15Rβ-2-GM-CSFRβ-2, IL-21R-1-IL-23R-2, IL-21R-1-GM-CSFRα, IL-21R-1-GM-CSFRβ-1, IL-21R-1-GM-CSFRβ-2, IL-21R-2-IL-23R-2, IL-21R-2-GM-CSFRα, IL-21R-2-GM-CSFRβ-1, IL-21R-2-GM-CSFRβ-2, IL-21R-3-IL-23R-2, IL-21R-3-GM-CSFRα, IL-21R-3-GM-CSFRβ-1, IL-21R-3-GM-CSFRβ-2, IL-23R-2-GM-CSFRα, IL-23R-2-GM-CSFRβ-1, IL-23R-2-GM-CSFRβ-2, IL-7Rα-IL-9R-2, IL12Rβ1-IL-9R-2, IL12Rβ2-IL-9R-2, IL15Rα-IL-9R-2, IL15Rβ-1-IL-9R-2, IL15Rβ-2-IL-9R-2, IL-21R-1-IL-9R-2, IL-21R-2-IL-9R-2, IL-21R-3-IL-9R-2, IL-23R-2-IL-9R-2, GM-CSFRα-IL-9R-2, GM-CSFRβ-1-IL-9R-2, GM-CSFRβ-2-IL-9R-2, IL-7Rα-IL-12Rβ2, or a variant thereof.

9. The immune effector of claim 7, wherein the cytokine receptor intracellular domain comprises an amino acid sequence selected from SEQ ID NOs: 8-28, or an amino acid sequence having at least 75%, 80%, 90%, 91%, 92%, 93%, 94%, 95%, 96%, 97%, 98%, or 99% identity to an amino acid sequence selected from SEQ ID NOs: 8-28.

10. The immune effector of claim 9, wherein the cytokine receptor intracellular domain comprises one or more the amino acid sequences of SEQ ID NOs: 8, 10, 14-17 or 20-28.

11. The immune effector cell of any one of claims 1 to 10, wherein the functional exogenous receptor is a T cell receptor (TCR), a chimeric antigen receptor (CAR), a chimeric TCR (cTCR), or a T cell antigen coupler (TAC)-like chimeric receptor.

12. The immune effector cell of claim 11, wherein the functional exogenous receptor is a CAR, wherein optionally the CAR is a single CAR, dual CAR, tandem CAR or split CAR.

13. The immune effector cell of claim 12, wherein the CAR binds to a tumor-associated antigen, wherein optionally the tumor-associated antigen is selected from the group consisting of GPC3, AFP, Claudin 18.2, CD19, CD20, CD22, BCMA, GD2, DLL3, MSLN, CD30, CLL1 and CD33.

14. The immune effector cell of claim 11, wherein the functional exogenous receptor is a TCR.

15. The immune effector cell of claim 14, wherein the TCR binds to tumor-associated antigen, wherein optionally the tumor-associated antigen is selected from the group consisting of GPC3, AFP, Claudin 18.2, CD19, CD20, CD22, BCMA, GD2, DLL3, MSLN, CD30, CLL1 or CD33.

16. The immune effector cell of any one of claims 1 to 15, wherein the second transmembrane domain is derived from a molecule selected from the group consisting of CD8, CD4, CD28, CD137, CD80, CD86, CD152 and PD1.

17. The immune effector cell of claim 16, wherein the second transmembrane domain is from CD8α or CD28.

18. The immune effector cell of any one of claims 1 to 17, wherein the intracellular signaling domain comprises a primary intracellular signaling domain of an immune effector cell.

19. The immune effector cell of claim 18, wherein the primary intracellular signaling domain is from CD3ζ.

20. The immune effector cell of any one of claims 1 to 19, wherein the intracellular signaling domain comprises a co-stimulatory signaling domain.

21. The immune effector cell of claim 20, wherein the co-stimulatory signaling domain is derived from a co-stimulatory molecule selected from the group consisting of CD27, CD28, CD137, OX40, CD30, CD40, CD3, LFA-1, ICOS, CD2, CD7, LIGHT, NKG2C, B7-H3, ligands of CD83 and combinations thereof.

22. The immune effector cell of claim 21, wherein the co-stimulatory signaling domain comprises a cytoplasmic domain of CD28 and/or a cytoplasmic domain of CD137.

23. The immune effector cell of any one of claims 1 to 22, wherein the functional exogenous receptor further comprises a hinge domain located between the C-terminus of the extracellular antigen binding domain and the N-terminus of the transmembrane domain.

24. The immune effector cell of claim 23, wherein the hinge domain is from CD8α.

25. The immune effector cell of any one of claims 1 to 24, wherein the chimeric cytokine receptor and/or the functional exogenous receptor further comprises a signal peptide.

26. The immune effector cell of claim 25, wherein the signal peptide is from CD8α.

27. The immune effector cell of any one of claims 12, 13 and 16 to 26, wherein the CAR comprises the amino acid sequence of SEQ ID NOs: 29, 135, 141, 143 or 149, or an amino acid sequence having at least 75%, 80%, 85%, 90%, 91%, 92%, 93%, 94%, 95%, 96%, 97%, 98%, or 99% identity to SEQ ID NOs: 29, 135, 141,143 or 149.

28. The immune effector cell of any one of claims 1 to 27, wherein the immune effector cell comprises the amino acid sequence of SEQ ID NOs: 30-134, 136-140, 142, 144, 146 or 148, or an amino acid sequence having at least 75%, 80%, 85%, 90%, 91%, 92%, 93%, 94%, 95%, 96%, 97%, 98%, or 99% identity to SEQ ID NOs: 30-134, 136-140, 142, 144, 146 or 148.

29. The immune effector cell of any one of claims 1 to 28, wherein the chimeric cytokine receptor further comprises a tag and/or the functional exogenous receptor further comprises a tag.

30. The immune effector cell of claim 29, where the tag linking to the chimeric cytokine receptor is different form the tag linking to the functional exogenous receptor.

31. The immune effector cell of claim 29 or 30, wherein the tag comprises an amino acid sequence SEQ ID NOs: 4, 5 or 158-160, or an amino acid sequence having at least 75%, 80%, 90%, 91%, 92%, 93%, 94%, 95%, 96%, 97%, 98%, or 99% identity to an amino acid sequence selected from SEQ ID NOs: 4, 5 or 158-160.

32. The immune effector cell of any one of claims 1 to 31, wherein the immune effector cell is a T cell, a natural killer (NK) cell, a NK T cell, a macrophage, a peripheral blood mononuclear cell (PBMC), a monocyte, a neutrophil, or an eosinophil.

33. The immune effector cell of claim 32, wherein the T cell is a cytotoxic T cell, a helper T cell, a natural killer T cell, a αβ T cell, or a γδT cell.

34. A polypeptide comprising:
(i) a chimeric cytokine receptor comprising:
(a) a first extracellular antigen binding domain, wherein the first extracellular antigen binding domain is derived from NKG2D, truncated NKG2D, antibody or antigen binding fragment thereof targeting NKG2D ligands, TIGIT or SIRP-α, or a variant thereof,
(b) a first transmembrane domain, and
(c) a cytokine receptor intracellular domain; and
(ii) optionally a functional exogenous receptor comprising:
(a) a second extracellular antigen binding domain,
(b) a second transmembrane domain, and
(c) an intracellular signaling domain.

35. The polypeptide of claim 34, wherein the first extracellular antigen binding domain binds to an antigen expressed on the surface of a tumor cell.

36. The polypeptide of claim 34 or claim 35,
(i) wherein the first extracellular antigen binding domain is derived from NKG2D, or truncated NKG2D, or a variant thereof, wherein optionally the first extracellular antigen binding domain is derived from the ECD of NKG2D or truncated NKG2D, and wherein optionally the first extracellular antigen binding domain comprises the amino acid sequence of SEQ ID NOs: 6, 156 or 157, or an amino acid sequence having at least 75%, 80%, 85%, 90%, 91%, 92%, 93%, 94%, 95%, 96%, 97%, 98%, or 99% identity to SEQ ID NOs: 6, 156 or 157;
(ii) wherein the first extracelluar antigen domain comprises an antibody or antigen binding fragment thereof that binds to an NKG2D ligand or a variant thereof, wherein optionally the NKG2D ligand is selected from a group consisting of MICA/B, ULBP-1, ULBP-2,5,6 and ULBP-3, and wherein optionally the antibody or antigen binding fragment thereof comprises one or more scFv(s) or one or more sdAb(s) that bind to the NKG2D ligand;
(iii) wherein the first extracelluar antigen domain is derived from TIGIT or a variant thereof, wherein optionally the first extracelluar antigen domain is derived from the ECD of TIGIT, and wherein optionally the first extracellular antigen binding domain comprises the amino acid sequence of SEQ ID NO: 145; or
(iv) wherein the first extracelluar antigen domain is derived from SIRP-α or a variant thereof, wherein optionally the first extracelluar antigen domain is derived from the ECD of SIRP-α, and wherein optionally the first extracellular antigen binding domain comprises the amino acid sequence of SEQ ID NO: 147.

37. The polypeptide of any one of claims 34 to 36, wherein the first transmembrane domain comprises a Janus Kinase (JAK)-binding domain.

38. The polypeptide of claim 37, wherein the JAK-binding domain is derived from a group consisting of EPOR, GHR, TPOR, or a variant thereof.

39. The polypeptide of claim 38, wherein the JAK-binding domain is derived from TPOR and comprises an amino acid sequence of SEQ ID NO: 7 or an amino acid sequence having at least 75%, 80%, 85%, 90%, 91%, 92%, 93%, 94%, 95%, 96%, 97%, 98%, or 99% identity to SEQ ID NO: 7.

40. The polypeptide of any one of claims 34 to 39, wherein the cytokine receptor intracellular domain is derived from one or more cytokine receptors selected from a group consisting of IL2Rα, IL7Rα, IL9R-1, TL9R-2, IL9R-3, IL12Rβ1, IL12Rβ2, IL15Rα, IL15Rβ-1, IL15Rβ-2, IL15Rβ-3, IL18Rα, IL18Rβ, IL21R-1, IL21R-2, IL21R-3, IL10R2, IL22R1, IL23R-1, IL23R-2, IL23R-3, IL27Rα, gp130, IL31RA, OSMRβ, TL36R, IL1RAcP, GM-CSFRα, GM-CSFRβ-1, GM-CSFRβ-2, or a variant thereof, and combinations thereof.

41. The polypeptide of claim 40, wherein the cytokine receptor intracellular domain is selected from IL-7Rα, IL12Rβ1, IL12Rβ2, IL15Rα, IL15Rβ-1, IL15Rβ-2, IL15Rβ-3, IL-18Rα, IL18Rβ, IL21R-1, IL21R-2, IL21R-3, IL23R-1, IL23R-2, IL23R-3, GM-CSFRα, GM-CSFRβ-1, GM-CSFRβ-2, IL9R-1, IL9R-2, IL9R-3, IL-7Rα-IL12Rβ1, IL-7Rα-IL15Rα, IL-7Rα-IL15Rβ-1, IL-7Rα-IL15Rβ-2, IL-7Rα-IL21R-1, IL-7Rα-IL21R-2, IL-7Rα-IL21R-3, IL-7Rα-IL23R-2, IL-7Rα-GMCSFRα, IL-7Rα-GM-CSFRβ-1, IL-7Rα-GM-CSFRβ-2, IL12Rβ1-IL15Rα, IL12Rβ1-IL15Rβ-1, IL12Rβ1-IL15Rβ-2, IL12Rβ1-IL-21R-1, IL12Rβ1-IL-21R-2, IL12Rβ1-IL-21R-3, IL12Rβ1-IL-23R-2, IL12Rβ1-GM-CSFRα, IL12Rβ1-GM-CSFRβ-1, IL12Rβ1-GM-CSFRβ-2, IL12Rβ2-IL15Rα, IL12Rβ2-IL15Rβ-1, IL12Rβ2-IL15Rβ-2, IL12Rβ2-IL-21R-1, IL12Rβ2-IL-21R-2, IL12Rβ2-IL-21R-3, IL12Rβ2-IL-23R-2, IL12Rβ2-GM-CSFRα, IL12Rβ2-GM-CSFRβ-1, IL12Rβ2-GM-CSFRβ-2, IL15Rα-IL-21R-1, IL15Rα-IL-21R-2, IL15Rα-IL-21R-3, IL15Rα-IL-23R-2, IL15Rα-GM-CSFRα, IL15Rα-GM-CSFRβ-1, IL15Rα-GM-CSFRβ-2, IL15Rβ-1-IL-21R-1, L15Rβ-1-IL-21R-2, IL15Rβ-1-IL-21R-3, IL15Rβ-1-IL-23R-2, IL15Rβ-1-GM-CSFRα, IL15Rβ-1-GM-CSFRβ-1, IL15Rβ-1-GM-CSFRβ-2, IL15Rβ-2-IL-21R-1, IL15Rβ-2-IL-21R-2, IL15Rβ-2-IL-21R-3, L15Rβ-2-IL-23R-2, IL15Rβ-2-GM-CSFRα, IL15Rβ-2-GM-CSFRβ-1, IL15Rβ-2-GM-CSFRβ-2, IL-21R-1-IL-23R-2, IL-21R-1-GM-CSFRα, IL-21R-1-GM-CSFRβ-1, IL-21R-1-GM-CSFRβ-2, IL-21R-2-IL-23R-2, IL-21R-2-GM-CSFRα, IL-21R-2-GM-CSFRβ-1, IL-21R-2-GM-CSFRβ-2, IL-21R-3-IL-23R-2, IL-21R-3-GM-CSFRα, IL-21R-3-GM-CSFRβ-1, IL-21R-3-GM-CSFRβ-2, IL-23R-2-GM-CSFRα, IL-23R-2-GM-CSFRβ-1, IL-23R-2-GM-CSFRβ-2, IL-7Rα-IL-9R-2, IL12Rβ1-IL-9R-2, IL12Rβ2-IL-9R-2, IL15Rα-IL-9R-2, IL15Rβ-1-IL-9R-2, IL15Rβ-2-IL-9R-2, IL-21R-1-IL-9R-2, IL-21R-2-IL-9R-2, IL-21R-3-IL-9R-2, IL-23R-2-IL-9R-2, GM-CSFRα-IL-9R-2, GM-CSFRβ-1-IL-9R-2, GM-CSFRβ-2-IL-9R-2, IL-7Rα-IL-12Rβ2, or a variant thereof.

42. The polypeptide of claim 41, wherein the cytokine receptor intracellular domain comprises an amino acid sequence selected from SEQ ID NOs: 8-28, or an amino acid sequence having at least 75%, 80%, 90%, 91%, 92%, 93%, 94%, 95%, 96%, 97%, 98%, or 99% identity to an amino acid sequence selected from SEQ ID NOs: 8-28.

43. The polypeptide of claim 42, wherein the cytokine receptor intracellular domain comprises one or more the amino acid sequences of SEQ ID NOs: 8, 10, 14-17 or 20-28.

44. The polypeptide of any one of claims 34 to 43, wherein the functional exogenous receptor is a T cell receptor (TCR), a chimeric antigen receptor (CAR), a chimeric TCR (cTCR), or a T cell antigen coupler (TAC)-like chimeric receptor.

45. The polypeptide of claim 44, wherein the functional exogenous receptor is a CAR, wherein optionally the CAR is a single CAR, dual CAR, tandem CAR or split CAR.

46. The polypeptide of claim 45, wherein the CAR binds to a tumor-associated antigen, wherein optionally the tumor-associated antigen is selected from the group consisting of GPC3, AFP, Claudin 18.2, CD19, CD20, CD22, BCMA, GD2, DLL3, MSLN, CD30, CLL1 and CD33.

47. The polypeptide of claim 44, wherein the functional exogenous receptor is a TCR.

48. The polypeptide of claim 47, wherein the TCR binds to tumor-associated antigen, wherein preferably the tumor-associated antigen is selected from the group consisting of GPC3, AFP, Claudin 18.2, CD19, CD20, CD22, BCMA, GD2, DLL3, MSLN, CD30, CLL1 and CD33.

49. The polypeptide of any one of claims 34 to 48, wherein the second transmembrane domain is derived from a molecule selected from the group consisting of CD8, CD4, CD28, CD137, CD80, CD86, CD152 and PD1.

50. The polypeptide of claim 49, wherein the second transmembrane domain is from CD8α or CD28.

51. The polypeptide of any one of claims 34 to 50, wherein the intracellular signaling domain comprises a primary intracellular signaling domain of an immune effector cell.

52. The polypeptide of claim 51, wherein the primary intracellular signaling domain is from CD3ζ.

53. The polypeptide of any one of claims 34 to 52, wherein the intracellular signaling domain comprises a co-stimulatory signaling domain.

54. The polypeptide of claim 53, wherein the co-stimulatory signaling domain is derived from a co-stimulatory molecule selected from the group consisting of CD27, CD28, CD137, OX40, CD30, CD40, CD3, LFA-1, ICOS, CD2, CD7, LIGHT, NKG2C, B7-H3, ligands of CD83 and combinations thereof.

55. The polypeptide of claim 54, wherein the co-stimulatory signaling domain comprises a cytoplasmic domain of CD28 and/or a cytoplasmic domain of CD137.

56. The polypeptide of any one of claims 34 to 55, wherein the functional exogenous receptor further comprises a hinge domain located between the C-terminus of the extracellular antigen binding domain and the N-terminus of the transmembrane domain.

57. The polypeptide of claim 56, wherein the hinge domain is from CD8α.

58. The polypeptide of any one of claims 34 to 57, wherein the chimeric cytokine receptor and/or the functional exogenous receptor further comprises a signal peptide.

59. The polypeptide of claim 58, wherein the signal peptide is from CD8α.

60. The polypeptide of any one of claims 34 to 59, wherein the chimeric cytokine receptor and the functional exogenous receptor are linked to each other via a peptide linker.

61. The polypeptide of claim 60, wherein the peptide linker is a self-cleaving peptide linker.

62. The polypeptide of claim 61, wherein the self-cleaving peptide linker is a 2A self-cleaving peptide.

63. The polypeptide of claim 62, wherein the 2A self-cleaving peptide is selected from a group consisting of F2A, E2A, P2A, T2A, and variants thereof.

64. The polypeptide of any one of claims 45, 46 and 49 to 63, wherein the CAR comprises the amino acid sequence of SEQ ID NOs: 29, 135, 141, 143 or 149, or an amino acid sequence having at least 75%, 80%, 85%, 90%, 91%, 92%, 93%, 94%, 95%, 96%, 97%, 98%, or 99% identity to SEQ ID NOs: 29, 135, 141, 143 or 149.

65. The polypeptide of any one of claims 34 to 64, wherein the immune effector cell comprises the amino acid sequence of SEQ ID NOs: 30-134, 136-140, 142, 144, 146 or 148, or an amino acid sequence having at least 75%, 80%, 85%, 90%, 91%, 92%, 93%, 94%, 95%, 96%, 97%, 98%, or 99% identity to SEQ ID NOs: 30-134, 136-140, 142, 144, 146 or 148.

66. The polypeptide of any one of claims 34 to 65, wherein the chimeric cytokine receptor further comprises a tag and/or the functional exogenous receptor further comprises a tag.

67. The polypeptide of claim 66, where the tag linking to the chimeric cytokine receptor is different form the tag linking to the functional exogenous receptor.

68. The polypeptide of claim 66, wherein the tag comprises an amino acid sequence SEQ ID NOs: 4, 5 or 158-160, or an amino acid sequence having at least 75%, 80%, 90%, 91%, 92%, 93%, 94%, 95%, 96%, 97%, 98%, or 99% identity to an amino acid sequence selected from SEQ ID NOs: 4, 5 or 158-160.

69. An isolated nucleic acid comprising a nucleic acid sequence encoding the polypeptide of any one of claims 34 to 68.

70. An isolated nucleic acid comprising:
(i) a first region encoding a chimeric cytokine receptor comprising:
(a) a first extracellular antigen binding domain, wherein the first extracellular antigen binding domain is derived from NKG2D, truncated NKG2D, antibody or antigen binding fragment thereof targeting NKG2D ligands, TIGIT, or SIRP-α, or a variant thereof,
(b) a first transmembrane domain, and
(c) a cytokine receptor intracellular domain; and
(ii) optionally a second region encoding a functional exogenous receptor comprising:
(a) a second extracellular antigen binding domain,
(b) a second transmembrane domain, and
(c) an intracellular signaling domain.

71. A vector comprising the nucleic acid of claim 69 or claim 70.

72. A method of making an immune effector cell comprising introducing into an immune cell:
(i) the nucleic acid of claim 69 or claim 70 or the vector of claim 71; or
(ii) a composition comprising a first nucleic acid encoding a chimeric cytokine receptor comprising (a) a first extracellular antigen binding domain, (b) a first transmembrane domain, and (c) a cytokine receptor intracellular domain; and a second nucleic acid encoding a functional exogenous receptor comprising (a) a second extracellular antigen binding domain, (b) a second transmembrane domain, and (c) an intracellular signaling domain.

73. An immune effector cell produced according the method of claim 72.

74. A pharmaceutical composition, comprising the immune effector cell of any one of claims 1 to 33 and 73, the polypeptide of any one of claims 34 to 68, the nucleic acid of claim 69 or claim 70, or the vector of claim 71, and a pharmaceutically acceptable carrier.

75. A method of treating a disease or disorder in a subject, comprising administering to the subject an effective amount of the pharmaceutical composition of claim 74.

76. The method of claim 75, wherein the disease or disorder is a cancer, an inflammatory or autoimmune disease.

77. The method of claim 76, wherein the cancer is solid cancer or hematologic cancer.

78. The method of claim 77, wherein the cancer is liver cancer, lymphoma, acute myeloid leukemia (AML) or chronic myelogenous leukemia (CML).
